SEQUENCE LISTING (190 sequences)

SEQ ID NO: 1 - protein, 107 aa, synthetic construct [sequence omitted]

SEQ ID NO: 2 - protein, 122 aa, synthetic construct [sequence omitted]

SEQ ID NO: 3 - protein, 11 aa, synthetic construct
RASQDIRNYLN

SEQ ID NO: 4 - protein, 8 aa, synthetic construct
YYTSRLES

SEQ ID NO: 5 - protein, 9 aa, synthetic construct
QQGNTLPWT

SEQ ID NO: 6 - protein, 5 aa, synthetic construct
GYTMN

SEQ ID NO: 7 - protein, 17 aa, synthetic construct
LINPYKGVSTYNQKFKD

SEQ ID NO: 8 - protein, 13 aa, synthetic construct
SGYYGDSDWYFDV

SEQ ID NO: 9 - protein, 115 aa, synthetic construct
  VARIANT 1: note = May be Q or G
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNI

SEQ ID NO: 10 - protein, 118 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 11 - protein, 6 aa, synthetic construct
DRGSQS

SEQ ID NO: 12 - protein, 6 aa, synthetic construct
IYSNGD

SEQ ID NO: 13 - protein, 13 aa, synthetic construct
AAVIDNDQGGILT

SEQ ID NO: 14 - protein, 5 aa, synthetic construct
PGHRA

SEQ ID NO: 15 - protein, 6 aa, synthetic construct
YVHGEE

SEQ ID NO: 16 - protein, 12 aa, synthetic construct
ASSPWDSPNVQY

SEQ ID NO: 17 - protein, 18 aa, synthetic construct
GGGSGGSGGCPPCGGSGG

SEQ ID NO: 18 - protein, 18 aa, synthetic construct
GGGSDDSGGCPPCGGKGG

SEQ ID NO: 19 - protein, 18 aa, synthetic construct
GGAAGGSGGCPPCGGSGG

SEQ ID NO: 20 - protein, 21 aa, synthetic construct
EPKSCDKTHTCPPCPAPELLG

SEQ ID NO: 21 - protein, 19 aa, synthetic construct
ERKCCVECPPCPAPPVAGP

SEQ ID NO: 22 - protein, 18 aa, synthetic construct
ESKYGPPCPSCPAPEFLG

SEQ ID NO: 23 - protein, 11 aa, synthetic construct
CPPCPAPELLG

SEQ ID NO: 24 - protein, 11 aa, synthetic construct
CPPCPAPEAAG

SEQ ID NO: 25 - protein, 220 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 26 - protein, 208 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 27 - protein, 220 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 28 - protein, 208 aa, synthetic construct
  VARIANT >208: note = May include further residues GK at C-terminus
PSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 29 - protein, 469 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 30 - protein, 473 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNGGGGSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 31 - protein, 470 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 32 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAK

SEQ ID NO: 33 - protein, 102 aa, synthetic construct
APPVAGPSVFLFPPKPKDTLMISRTPEVTCWVDVSHEDPEVKFNWYVDGVEVHNAKTKPREEQYQSTYRVVSVLTVLHQDWLNGKEYKCKVSNKALPASIEK

SEQ ID NO: 34 - protein, 101 aa, synthetic construct
APPVAGPSVFLFPPKPKDTLMISRTPEVTCWVDVSHEDPEVKFNWYVDGVEVHNAKTKPREEQYNSTYRWSVLTVLHQDWLNGKEYKCKVSNKALPASIEK

SEQ ID NO: 35 - protein, 479 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 463 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 12 aa, synthetic construct
GSAGSAAGSGEF

SEQ ID NO: 38 - protein, 18 aa, synthetic construct
KESGSVSSEQLAQFRSLD

SEQ ID NO: 39 - protein, 14 aa, synthetic construct
EGKSSGSGSESKST

SEQ ID NO: 40 - protein, 8 aa, synthetic construct
GGGSGGGG

SEQ ID NO: 41 - protein, 4 aa, synthetic construct
GGGS

SEQ ID NO: 42 - protein, 5 aa, synthetic construct
GGGGS

SEQ ID NO: 43 - protein, 5 aa, synthetic construct
TVLRT

SEQ ID NO: 44 - protein, 6 aa, synthetic construct
TVSSAS

SEQ ID NO: 45 - protein, 7 aa, synthetic construct
TVLSSAS

SEQ ID NO: 46 - protein, 20 aa, synthetic construct
GGGGSGGGGSGGGGSGGGGS

SEQ ID NO: 47 - protein, 17 aa, synthetic construct
GSADDAKKDAAKKDGKS

SEQ ID NO: 48-51 - skipped (4 entries)

SEQ ID NO: 52 - protein, 4 aa, synthetic construct
CPPC

SEQ ID NO: 53 - protein, 4 aa, synthetic construct
CGPC

SEQ ID NO: 54 - protein, 4 aa, synthetic construct
CPGC

SEQ ID NO: 55 - protein, 4 aa, synthetic construct
CGGC

SEQ ID NO: 56 - protein, 4 aa, synthetic construct
CSPG

SEQ ID NO: 57 - protein, 4 aa, synthetic construct
CPSC

SEQ ID NO: 58 - protein, 4 aa, synthetic construct
CSSC

SEQ ID NO: 59 - protein, 4 aa, synthetic construct
CGSC

SEQ ID NO: 60 - protein, 4 aa, synthetic construct
CSGC

SEQ ID NO: 61 - protein, 5 aa, synthetic construct
CPPPC

SEQ ID NO: 62 - protein, 5 aa, synthetic construct
CGPPC

SEQ ID NO: 63 - protein, 5 aa, synthetic construct
CPGPC

SEQ ID NO: 64 - protein, 5 aa, synthetic construct
CPPGC

SEQ ID NO: 65 - protein, 5 aa, synthetic construct
CGGPC

SEQ ID NO: 66 - protein, 5 aa, synthetic construct
CPGGC

SEQ ID NO: 67 - protein, 5 aa, synthetic construct
CGGGC

SEQ ID NO: 68 - protein, 5 aa, synthetic construct
CSPPC

SEQ ID NO: 69 - protein, 5 aa, synthetic construct
CPSPC

SEQ ID NO: 70 - protein, 5 aa, synthetic construct
CPPSC

SEQ ID NO: 71 - protein, 5 aa, synthetic construct
CSSPC

SEQ ID NO: 72 - protein, 5 aa, synthetic construct
CPSSC

SEQ ID NO: 73 - protein, 5 aa, synthetic construct
CSSSC

SEQ ID NO: 74 - protein, 5 aa, synthetic construct
CGSPC

SEQ ID NO: 75 - protein, 5 aa, synthetic construct
CPGSC

SEQ ID NO: 76 - protein, 5 aa, synthetic construct
CSGPC

SEQ ID NO: 77 - protein, 5 aa, synthetic construct
CPSGC

SEQ ID NO: 78 - protein, 12 aa, synthetic construct
CPPCPAPEAAGG

SEQ ID NO: 79 - protein, 17 aa, synthetic construct
DKTHTCPPCPAPEAAGG

SEQ ID NO: 80 - protein, 22 aa, synthetic construct
EPKSSDKTHTCPPCPAPEAAGG

SEQ ID NO: 81 - protein, 9 aa, synthetic construct
SLLQHLIGL

SEQ ID NO: 82 - protein, 6 aa, synthetic construct
ELLGGP

SEQ ID NO: 83 - protein, 475 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNGSADDAKKDAAKKDGKSQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 84 - protein, 478 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNGGGGSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 85 - protein, 471 aa, synthetic construct
KAGVTQTPRYLIKTRGQQVTLSCSPIPGHRAVSWYQQTPCQGLQFLFEYVHGEERNKGNFPGRFSGRQFSNSSSEMNISNLELGDSALYLCASSPWDSPNVQYFGPGTRLTVTEDLKNGGGSGGSGGCPPCGGSGGGKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGCGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 86 - protein, 471 aa, synthetic construct
KAGVTQTPRYLIKTRGQQVTLSCSPIPGHRAVSWYQQTPCQGLQFLFEYVHGEERNKGNFPGRFSGRQFSNSSSEMNISNLELGDSALYLCASSPWDSPNVQYFGPGTRLTVTEDLKNGGGSDDSGGCPPCGGKGGGKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGCGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 87 - protein, 475 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGGSGGGGSGGGGSGGGGSKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 88 - protein, 473 aa, synthetic construct
QKEVEQNSGPLSVPEGAIASLNCTYSDRGSQSFFWYRQYSGCSPELIMSIYSNGDKEDGRFTAQLNKASQYVSLLIRDSQPSDSATYLCAAVIDNDQGGILTFGTGTRLTIIPNIQNGGGSGGSGGCPPCGGSGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGCGTRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 89 - protein, 473 aa, synthetic construct
QKEVEQNSGPLSVPEGAIASLNCTYSDRGSQSFFWYRQYSGCSPELIMSIYSNGDKEDGRFTAQLNKASQYVSLLIRDSQPSDSATYLCAAVIDNDQGGILTFGTGTRLTIIPNIQNGGAAGGSGGCPPCGGSGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGCGTRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 90 - protein, 473 aa, synthetic construct
KAGVTQTPRYLIKTRGQQVTLSCSPIPGHRAVSWYQQTPGQCLQFLFEYVHGEERNKGNFPGRFSGRQFSNSSSEMNISNLELGDSALYLCASSPWDSPNVQYFGPGTRLTVTEDLKNGGGGSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGCGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 91 - protein, 483 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNGGGGSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 92 - protein, 470 aa, synthetic construct
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNGGGGSGGGGSGGGGSGGGGSQKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 93 - protein, 468 aa, synthetic construct
EAGVTQSPTHLIKTRGQQVTLRCSPKSGHDTVSWYQQALCQGPQFIFQYVRGEERQRGNFPDRFSGHQYPNYSSELNINALLLGDSALYLCASSDTVSYEQYFGPGIRLTVTEDLKNGGGSGGSGGCPPCGGSGGGKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGCGTSLLVTPHICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 94 - protein, 472 aa, synthetic construct
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHIQNGGGGSGGGGSGGGGSGGGGSEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 95 - protein, 470 aa, synthetic construct
QKEVEQNSGPLSVPEGAIASLNCTYSDRGSQSFFWYRQYSGCSPELIMSIYSNGDKEDGRFTAQLNKASQYISLLIRDSKLSDSATYLCAVRGAHDYALNFGKGTSLLVTPHIQNGGGSGGSGGCPPCGGSGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGCGIRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 96 - protein, 470 aa, synthetic construct
NAGVTQTPKFQVLKTGQSMTLQCAQDMNHEYMAWYRQDPGMGLRLIHYSVGVGITDQGDVPDGYKVSRSTTEDFPLRLLSAAPSQTSVYFCASRPGLMSAQPELYFGPGTRLTVTEDLINGSADDAKKDAAKKDGKSQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 97 - protein, 473 aa, synthetic construct
NAGVTQTPKFQVLKTGQSMTLQCAQDMNHEYMAWYRQDPGMGLRLIHYSVGVGITDQGDVPDGYKVSRSTTEDFPLRLLSAAPSQTSVYFCASRPGLMSAQPELYFGPGTRLTVTEDLINGGGGSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 98 - protein, 471 aa, synthetic construct
NAGVTQTPKFQVLKTGQSMTLQCAQDMNHEYMAWYRQDPCMGLRLIHYSVGVGITDQGDVPDGYKVSRSTTEDFPLRLLSAAPSQTSVYFCASRPGLMSAQPELYFGPGTRLTVTEDLINGGGSGGSGGCPPCGGSGGGKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGCGTQVVVTPDICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 99 - protein, 475 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNGGGGSGGGGSGGGGSGGGGSNAGVTQTPKFQVLKTGQSMTLQCAQDMNHEYMAWYRQDPGMGLRLIHYSVGVGITDQGDVPDGYKVSRSTTEDFPLRLLSAAPSQTSVYFCASRPGLMSAQPELYFGPGTRLTVTEDLINCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 100 - protein, 473 aa, synthetic construct
QKEVEQNSGPLSVPEGAIASLNCTYSDRGSQSFFWYRQYSGCSPELIMSIYSNGDKEDGRFTAQLNKASQYVSLLIRDSQPSDSATYLCAVTTDSWGKLQFGAGTQVVVTPDIQNGGGSGGSGGCPPCGGSGGNAGVTQTPKFQVLKTGQSMTLQCAQDMNHEYMAWYRQDPGMGLRLIHYSVGVGITDQGDVPDGYKVSRSTTEDFPLRLLSAAPSQTSVYFCASRPGLMSAQPELYFGCGTRLTVTEDLINCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 101 - protein, 469 aa, synthetic construct
DAGVIQSPRHEVTEMGQEVTLRCKPIPGHDYLFWYRQTMMRGLELLFYFCYGTPCDDSGMPEDRFSAKMPNASFSTLKIQPSEPRDSAVYFCASRADTGELFFGEGSRLTVLEDLKNGGGGSGGGGSGGGGSGGGGSQEDVEQSLFLSVREGDSVVINCTYTDSSSTYLYWYKQEPGKGLQLLTYIYSSQDQKQDQRLTVLLNKKDKHLSLRIADTQTGDSAIYFCAEMTSESKIIFGSGTRLSIRPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 102 - protein, 467 aa, synthetic construct
DAGVIQSPRHEVTEMGQEVTLRCKPIPGHDYLFWYRQTMCRGLELLFYFCYGTPCDDSGMPEDRFSAKMPNASFSTLKIQPSEPRDSAVYFCASRADTGELFFGEGSRLTVLEDLKNGGGSGGSGGCPPCGGSGGGEDVEQSLFLSVREGDSVVINCTYTDSSSTYLYWYKQEPGKGLQLLTYIYSSQDQKQDQRLTVLLNKKDKHLSLRIADTQTGDSAIYFCAEMTSESKIIFGCGTRLSIRPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 103 - protein, 471 aa, synthetic construct
GEDVEQSLFLSVREGDSVVINCTYTDSSSTYLYWYKQEPGKGLQLLTYIYSSQDQKQDQRLTVLLNKKDKHLSLRIADTQTGDSAIYFCAEMTSESKIIFGSGTRLSIRPNIQNGGGGSGGGGSGGGGSGGGGSDAGVIQSPRHEVTEMGQEVTLRCKPIPGHDYLFWYRQTMMRGLELLFYFCYGTPCDDSGMPEDRFSAKMPNASFSTLKIQPSEPRDSAVYFCASRADTGELFFGEGSRLTVLEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 104 - protein, 469 aa, synthetic construct
GEDVEQSLFLSVREGDSVVINCTYTDSSSTYLYWYKQEPGCGLQLLTYIYSSQDQKQDQRLTVLLNKKDKHLSLRIADTQTGDSAIYFCAEMTSESKIIFGSGTRLSIRPNIQNGGGSGGSGGCPPCGGSGGDAGVIQSPRHEVTEMGQEVTLRCKPIPGHDYLFWYRQTMMRGLELLFYFCYGTPCDDSGMPEDRFSAKMPNASFSTLKIQPSEPRDSAVYFCASRADTGELFFGCGSRLTVLEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 105 - protein, 474 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 106 - protein, 479 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 107 - protein, 453 aa, synthetic construct
EDVEQSLFLSVREGDSVVINCTYTDSSSTYLYWYKQEPGKGLQLLTYIYSSQDQKQDQRLTVLLNKKDKHLSLRIADTQTGDSAIYFCAEMTSESKIIFGSGTRLSIRPGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 108 - protein, 471 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGDAGVIQSPRHEVTEMGQEVTLRCKPIPGHDYLFWYRQTMMRGLELLFYFCYGTPCDDSGMPEDRFSAKMPNASFSTLKIQPSEPRDSAVYFCASRADTGELFFGEGSRLTVLEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 109 - skipped

SEQ ID NO: 110 - protein, 5 aa, synthetic construct
DRGSQ

SEQ ID NO: 111 - protein, 4 aa, synthetic construct
IYSN

SEQ ID NO: 112 - protein, 7 aa, synthetic construct
RGAHDYA

SEQ ID NO: 113 - protein, 5 aa, synthetic construct
KSGHD

SEQ ID NO: 114 - protein, 5 aa, synthetic construct
VRGEE

SEQ ID NO: 115 - protein, 5 aa, synthetic construct
DTVSY

SEQ ID NO: 116 - protein, 5 aa, synthetic construct
DRGSQ

SEQ ID NO: 117 - protein, 4 aa, synthetic construct
IYSN

SEQ ID NO: 118 - protein, 7 aa, synthetic construct
TTDSWGK

SEQ ID NO: 119 - protein, 5 aa, synthetic construct
DMNHE

SEQ ID NO: 120 - protein, 5 aa, synthetic construct
VGVGI

SEQ ID NO: 121 - protein, 8 aa, synthetic construct
PGLMSAQP

SEQ ID NO: 122 - protein, 5 aa, synthetic construct
DSSST

SEQ ID NO: 123 - protein, 5 aa, synthetic construct
YIYSS

SEQ ID NO: 124 - protein, 8 aa, synthetic construct
MTSESKII

SEQ ID NO: 125 - protein, 5 aa, synthetic construct
IPGHD

SEQ ID NO: 126 - protein, 6 aa, synthetic construct
CYGTPC

SEQ ID NO: 127 - protein, 9 aa, synthetic construct
ADTGELFFG

SEQ ID NO: 128 - protein, 9 aa, synthetic construct
SLYNTVATL

SEQ ID NO: 129 - protein, 9 aa, synthetic construct
LLFGYPVYV

SEQ ID NO: 130 - protein, 10 aa, synthetic construct
KVLEHVVRVI

SEQ ID NO: 131 - protein, 4 aa, synthetic construct
  DISULFID 3..4: note = intrachain disulfide bond
NICC

SEQ ID NO: 132 - protein, 14 aa, synthetic construct
  DISULFID 8..9: note = intrachain disulfide bond
NIDKTHTCCTHTKD

SEQ ID NO: 133 - protein, 24 aa, synthetic construct
  DISULFID 13..14: note = intrachain disulfide bond
NIEPKSSDKTHTCCTHTKDSSKPE

SEQ ID NO: 134 - protein, 479 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 135 - protein, 463 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 473 aa, synthetic construct
QKEVEQNSGPLSVPEGAIASLNCTYSDRGSQSFFWYRQYSCKSPELIMSIYSNGDKEDGRFTAQLNKASQYVSLLIRDSQPSDSATYLCAAVIDNDQGGILTFGTGTRLTIIPNIQNGGGSGGSGGCPPCGGSGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGCGTRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 137 - protein, 473 aa, synthetic construct
QKEVEQNSGPLSVPEGAIASLNCTYSDRGSQSFFWYRQYSCKSPELIMSIYSNGDKEDGRFTAQLNKASQYVSLLIRDSQPSDSATYLCAAVIDNDQGGILTFGTGTRLTIIPNIQNGGAAGGSGGCPPCGGSGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGCGTRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 138 - protein, 470 aa, synthetic construct
QKEVEQNSGPLSVPEGAIASLNCTYSDRGSQSFFWYRQYSCKSPELIMSIYSNGDKEDGRFTAQLNKASQYISLLIRDSKLSDSATYLCAVRGAHDYALNFGKGTSLLVTPHIQNGGGSGGSGGCPPCGGSGGEAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGCGIRLTVTEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 139 - protein, 473 aa, synthetic construct
QKEVEQNSGPLSVPEGAIASLNCTYSDRGSQSFFWYRQYSCKSPELIMSIYSNGDKEDGRFTAQLNKASQYVSLLIRDSQPSDSATYLCAVTTDSWGKLQFGAGTQVVVTPDIQNGGGSGGSGGCPPCGGSGGNAGVTQTPKFQVLKTGQSMTLQCAQDMNHEYMAWYRQDPGMGLRLIHYSVGVGITDQGDVPDGYKVSRSTTEDFPLRLLSAAPSQTSVYFCASRPGLMSAQPELYFGCGTRLTVTEDLINCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 140 - protein, 469 aa, synthetic construct
GEDVEQSLFLSVREGDSVVINCTYTDSSSTYLYWYKQEPCKGLQLLTYIYSSQDQKQDQRLTVLLNKKDKHLSLRIADTQTGDSAIYFCAEMTSESKIIFGSGTRLSIRPNIQNGGGSGGSGGCPPCGGSGGDAGVIQSPRHEVTEMGQEVTLRCKPIPGHDYLFWYRQTMMRGLELLFYFCYGTPCDDSGMPEDRFSAKMPNASFSTLKIQPSEPRDSAVYFCASRADTGELFFGCGSRLTVLEDLKNCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 141 - protein, 471 aa, synthetic construct
KAGVTQTPRYLIKTRGQQVTLSCSPIPGHRAVSWYQQTCGQGLQFLFEYVHGEERNKGNFPGRFSGRQFSNSSSEMNISNLELGDSALYLCASSPWDSPNVQYFGPGTRLTVTEDLKNGGGSGGSGGCPPCGGSGGGKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGCGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 142 - protein, 471 aa, synthetic construct
KAGVTQTPRYLIKTRGQQVTLSCSPIPGHRAVSWYQQTCGQGLQFLFEYVHGEERNKGNFPGRFSGRQFSNSSSEMNISNLELGDSALYLCASSPWDSPNVQYFGPGTRLTVTEDLKNGGGSDDSGGCPPCGGKGGGKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGCGTRLTIIPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 143 - protein, 468 aa, synthetic construct
EAGVTQSPTHLIKTRGQQVTLRCSPKSGHDTVSWYQQACGQGPQFIFQYVRGEERQRGNFPDRFSGHQYPNYSSELNINALLLGDSALYLCASSDTVSYEQYFGPGIRLTVTEDLKNGGGSGGSGGCPPCGGSGGGKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGCGTSLLVTPHICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 144 - protein, 471 aa, synthetic construct
NAGVTQTPKFQVLKTGQSMTLQCAQDMNHEYMAWYRQDCGMGLRLIHYSVGVGITDQGDVPDGYKVSRSTTEDFPLRLLSAAPSQTSVYFCASRPGLMSAQPELYFGPGTRLTVTEDLINGGGSGGSGGCPPCGGSGGGKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGCGTQVVVTPDICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 145 - protein, 467 aa, synthetic construct
DAGVIQSPRHEVTEMGQEVTLRCKPIPGHDYLFWYRQTCMRGLELLFYFCYGTPCDDSGMPEDRFSAKMPNASFSTLKIQPSEPRDSAVYFCASRADTGELFFGEGSRLTVLEDLKNGGGSGGSGGCPPCGGSGGGEDVEQSLFLSVREGDSVVINCTYTDSSSTYLYWYKQEPGKGLQLLTYIYSSQDQKQDQRLTVLLNKKDKHLSLRIADTQTGDSAIYFCAEMTSESKIIFGCGTRLSIRPNICPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 146 - protein, 7 aa, synthetic construct
YSGCSPE

SEQ ID NO: 147 - protein, 7 aa, synthetic construct
EPGCGLQ

SEQ ID NO: 148 - protein, 7 aa, synthetic construct
YFGCGTR

SEQ ID NO: 149 - protein, 7 aa, synthetic construct
FFGCGSR

SEQ ID NO: 150 - protein, 7 aa, synthetic construct
YSCKSPE

SEQ ID NO: 151 - protein, 7 aa, synthetic construct
EPCKGLQ

SEQ ID NO: 152 - protein, 7 aa, synthetic construct
YFGCGTR

SEQ ID NO: 153 - protein, 7 aa, synthetic construct
FFGCGSR

SEQ ID NO: 154 - protein, 7 aa, synthetic construct
QTPCQGL

SEQ ID NO: 155 - protein, 7 aa, synthetic construct
QALCQGP

SEQ ID NO: 156 - protein, 7 aa, synthetic construct
QDPCMGL

SEQ ID NO: 157 - protein, 7 aa, synthetic construct
QTMCRGL

SEQ ID NO: 158 - protein, 7 aa, synthetic construct
TFGCGTR

SEQ ID NO: 159 - protein, 7 aa, synthetic construct
NFGCGTS

SEQ ID NO: 160 - protein, 7 aa, synthetic construct
QFGCGTQ

SEQ ID NO: 161 - protein, 7 aa, synthetic construct
IFGCGTR

SEQ ID NO: 162 - protein, 7 aa, synthetic construct
QTCGQGL

SEQ ID NO: 163 - protein, 7 aa, synthetic construct
QACGQGP

SEQ ID NO: 164 - protein, 7 aa, synthetic construct
QDCGMGL

SEQ ID NO: 165 - protein, 7 aa, synthetic construct
QTCMRGL

SEQ ID NO: 166 - protein, 7 aa, synthetic construct
QTCMRGL

SEQ ID NO: 167 - protein, 107 aa, synthetic construct
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIK

SEQ ID NO: 168 - protein, 120 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTV

SEQ ID NO: 169 - protein, 113 aa, synthetic construct
  VARIANT 1: note = May be Q or G
QKEVEQNSGPLSVPEGAIASLNCTYSDRGSQSFFWYRQYSGKSPELIMSIYSNGDKEDGRFTAQLNKASQYISLLIRDSKLSDSATYLCAVRGAHDYALNFGKGTSLLVTPHI

SEQ ID NO: 170 - protein, 117 aa, synthetic construct
EAGVTQSPTHLIKTRGQQVTLRCSPKSGHDTVSWYQQALGQGPQFIFQYVRGEERQRGNFPDRFSGHQYPNYSSELNINALLLGDSALYLCASSDTVSYEQYFGPGIRLTVTEDLKN

SEQ ID NO: 171 - protein, 113 aa, synthetic construct
  VARIANT 1: note = May be Q or G
QKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDI

SEQ ID NO: 172 - protein, 120 aa, synthetic construct
NAGVTQTPKFQVLKTGQSMTLQCAQDMNHEYMAWYRQDPGMGLRLIHYSVGVGITDQGDVPDGYKVSRSTTEDFPLRLLSAAPSQTSVYFCASRPGLMSAQPELYFGPGTRLTVTEDLIN

SEQ ID NO: 173 - protein, 112 aa, synthetic construct
  VARIANT 1: note = May be Q or G
QEDVEQSLFLSVREGDSVVINCTYTDSSSTYLYWYKQEPGKGLQLLTYIYSSQDQKQDQRLTVLLNKKDKHLSLRIADTQTGDSAIYFCAEMTSESKIIFGSGTRLSIRPNI

SEQ ID NO: 174 - protein, 117 aa, synthetic construct
DAGVIQSPRHEVTEMGQEVTLRCKPIPGHDYLFWYRQTMMRGLELLFYFCYGTPCDDSGMPEDRFSAKMPNASFSTLKIQPSEPRDSAVYFCASRADTGELFFGEGSRLTVLEDLKN

SEQ ID NO: 175 - skipped

SEQ ID NO: 176 - protein, 4 aa, synthetic construct
  DISULFID 2..3: note = intrachain disulfide bond
TCCT

SEQ ID NO: 177 - protein, 6 aa, synthetic construct
  DISULFID 3..4: note = intrachain disulfide bond
HTCCTH

SEQ ID NO: 178 - protein, 8 aa, synthetic construct
  DISULFID 4..5: note = intrachain disulfide bond
THTCCTHT

SEQ ID NO: 179 - protein, 10 aa, synthetic construct
  DISULFID 5..6: note = intrachain disulfide bond
KTHTCCTHTK

SEQ ID NO: 180 - protein, 12 aa, synthetic construct
  DISULFID 6..7: note = intrachain disulfide bond
DKTHTCCTHTKD

SEQ ID NO: 181 - protein, 22 aa, synthetic construct
  DISULFID 11..12: note = intrachain disulfide bond
EPKSSDKTHTCCTHTKDSSKPE

SEQ ID NO: 182-184 - skipped (3 entries)

SEQ ID NO: 185 - protein, 4 aa, synthetic construct
NIQN

SEQ ID NO: 186 - protein, 4 aa, synthetic construct
HIQN

SEQ ID NO: 187 - protein, 4 aa, synthetic construct
DIQN

SEQ ID NO: 188 - skipped

SEQ ID NO: 189 - protein, 5 aa, synthetic construct
EDLKN

SEQ ID NO: 190 - protein, 5 aa, synthetic construct
EDLIN

(19) United States
(12) Patent Application Publication
(10) Pub. No.: US 2026/0234265 A1
(43) Pub. Date: Aug. 13, 2026

(54) BISPECIFIC POLYPEPTIDE MOLECULE

(71) Applicant: Enara Bio Limited, Oxford (GB)

(72) Inventors: Magdalena VON ESSEN, Oxford (GB); Duncan HOWIE, Oxford (GB); Peter MASON, Wilmington, DE (US); Emily LAM, Oxford (GB); Terri CORNFORTH, Oxford (GB)

(21) Appl. No.: 19/438,943

(22) Filed: Jan. 2, 2026

Related U.S. Application Data
(63) Continuation of application No. PCT/GB2024/051771, filed on Jul. 5, 2024.
(60) Provisional application No. 63/512,019, filed on Jul. 5, 2023.

(51) Int. Cl. C07K 16/28; A61P 35/00

(52) U.S. Cl. CPC C07K 16/2833; A61P 35/00; C07K 2317/31; C07K 2317/622; C07K 2317/92

(57) ABSTRACT

The present invention relates to bispecific polypeptide molecules capable of simultaneously binding to a disease associated antigen and a cell surface antigen expressed on the surface of an immune effector cell such as a T-cell resulting in the activation of the immune effector cell.

### RELATED APPLICATIONS

This application is a continuation of International Application Number PCT/GB2024/051771, filed Jul. 5, 2024, which claims priority to U.S. Provisional Application No. 63/512,019, filed Jul. 5, 2023, the entire disclosures of which are hereby incorporated by reference herein.

### SEQUENCE LISTING

This application contains a sequence listing which has been submitted electronically in XML format and is hereby incorporated by reference in its entirety (said XML copy, created Jul. 4, 2024, is named “360499-2024_SeqList_ST26.xml” and is 186,240 bytes in size).

### FIELD OF THE INVENTION

The present invention relates to bispecific polypeptide molecules capable of simultaneously binding to a disease associated antigen and a cell surface antigen expressed on the surface of an immune effector cell such as a T-cell resulting in the activation of the immune effector cell. The invention further relates to polynucleotides encoding the bispecific polypeptide molecules, and vectors and host cells containing these nucleic acids.

The invention further relates to methods for producing the bispecific polypeptide molecules, and methods of their use in the treatment of disease.

### BACKGROUND OF THE INVENTION

There has been a focus in the field of cancer therapy over the past several decades on the development of bispecific molecules which are dual activity molecules combing a first binding domain specific for an epitope on tumour cells and a second binding domain specific for an epitope on immune effector cells. Various arrangements of binding domains have been proposed with the intention being to redirect the activity of immune effector cells to the site of tumour and to offer promising immune-stimulatory agents to treat cancer.

Different formats have been proposed for bispecific molecules, including formats with or without IgG Fc regions, combining symmetric or asymmetric designs based on IgG-derived or TCR-derived component parts. Epitope binding regions of such bispecific molecules generally incorporate either antibody or TCR derived binding domains.

The discovery and production of single-chain connected variable domains of antibodies (scFvs) led to the development of bispecific antibody derived molecules like the BiTE®. BiTE molecules, such as Blinatumomab, a BiTE targeting CD19, (Baeuerle, P. A.; Reinhardt, C. “bispecific T-cell engaging antibodies for cancer therapy”, Cancer Res. 2009, 69, 4941-4944) have been promoted for cancer therapy. These molecules co-engage the CD3E subunit on T cells and a surface antigen on the tumour cell and thereby triggerT cell-mediated killing of the tumour. Concurrent engagement of the target cell antigen and CD3 leads to activation of polyclonal cytotoxic T-cells, resulting in target cell lysis. Being a small bispecific molecule, the BiTE, has a very short serum half-life, its size also makes it difficult to produce and purify due to aggregation. The chains of BiTEs antibody derived binding domains are connected by internal linker molecules, providing flexibility to the construct and favourable antigen-binding kinetics for the specific antigens targeted. Dual-affinity re-targeting molecules (DARTs) have a similar basic structure but include a disulphide linker for additional inter-chain stability. Bispecific (BiKEs) and trispecific killer cell engagers (TriKEs) consist of either two (BiKE) or three (TriKE) variable antigen binding regions and activate natural killer cells either by binding to CD16 and optionally containing an IL15 cross-linker [Allen, C., Life 2021, 11(6), 465]. DART molecules proved highly effective at redirected T-cell killing of B-cell lymphoma. DART molecules proved to be more potent in directing B-cell lysis than a single-chain, bispecific antibody bearing identical CD19 and CD3 antibody Fv sequences.

Use of IgG Fc in bispecific constructs has been employed to overcome issues of short serum half life. Originally flexible linker peptides were fused to the C termini of the heavy chains of IgG to permit the attachment of single-chain variable domains with different binding specificities to form a tetravalent bispecifics with increased productivity levels and more simplified purification (Coloma, M. J. and Morrison, S. L. (1997), “design and production of novel tetravalent bispecific antibodies”, Nat. Biotechnol. 15, 159-163). For example, the addition of Fc to the Anti-P-cadherin/Anti-CD3 Bispecific DART Molecule to form DART-Fc-achieved a significantly extended half-life whilst maintaining high potency for its cancer target.

IgG-based bispecific formats have been generally improved by incorporation of engineered Fc mutations to facilitate the hetero-dimerization of two differing CH3-domains thereby connecting two different polypeptide chains which may have different binding functions optimally combined into the one therapeutic molecule; (Ridgway J B, et al. ‘Knobs-into-holes’ engineering of antibody CH3 domains for heavy chain heterodimerization. Protein Eng. 1996 July; 9(7):617-21). This Fc format was further improved by the additional introduction of cysteine-residues to form a stabilising disulphide-bond between the heterodimeric CH3-domains.

Inclusion of the Fc into a bispecific format provided the advantage of accessing an interaction of the Fc-part of the bispecific with the human Fc-receptor FcRn. This prolonged the half-life of the IgG format through interaction with neonatal FcRn. Further Fc mutations were developed to ensure that the recombinant molecule is capable of binding the target molecule without triggering significant complement dependent lysis, or cell mediated destruction of the target, mitigating against adverse reactions in therapy. It was also necessary to produce variants which prevent FcgR-binding by the Fc domain (and particularly abolish FcgRI binding, C1q binding and FcgRIII binding) in order to prevent off-target activation of immune effector cells.

Bispecifics comprising T-cell receptor (TCR) binding domains have been successfully developed as therapeutic molecules. TCRs comprise two chains, alpha and beta, each with variable and constant domains, where the variable domains are involved in binding antigenic peptide in the form of a peptide-MHC complex. For example, WO 2016/184592 discloses bispecific molecules in which one specificity is contributed by a TCR and the other by an antibody, which is directed against an antigen or epitope on the surface of lymphocytes.

The TCR binding domain may consist of a single-chain TCR (scTCRs) i.e. engineered soluble polypeptide constructs of the alpha and beta chain connected by a linker domain, these afford significant advantages in contrast to the full-length TCR format for engineering, soluble protein expression, and clinical potential. One option of TCR binding domain is the single-chain TCR (scTCR) which is produced as a single polypeptide, avoiding the requirement for production of each TCR chain as separate polypeptides and allowing for production of larger quantities of the properly assembled scTCR that binds to its peptide-MHC ligand. US 2006-0166875 discloses a single chain T cell receptor (scTCR) construct which can consist of one or two or more scTCR units. The single chain T cell receptor construct comprises a segment constituted by a TCR alpha chain variable region sequence fused to the N terminus of a TCR alpha chain constant region extracellular sequence, a beta segment constituted by a TCR beta chain variable region fused to the N terminus of a TCR beta chain constant region extracellular sequence, and a linker sequence linking the C terminus of the alpha segment to the N terminus of the beta segment, or vice-versa. The constant region extracellular sequences of the alpha and beta segments being linked by a disulphide a bond, the length of the linker sequence and the position of the disulphide bond being such that the variable region sequences of the alpha and beta segments are mutually orientated substantially as in native alpha/beta T cell receptors. scTCRs consisting of only the V regions (Valpha-linker-Vbeta) are denoted scTv and offer an alternative suitable format as a therapeutic or diagnostic reagent (see US 20060166875, US 20120252742).

On example of a TCR containing bispecific construct is the bifunctional ImmTAC, (McCormack E, et al. Cancer Immunol Immunother. 2013 April; 62(4):773-85), comprising a soluble, high-affinity T-cell receptor (TCR) specific for HLA-A*0201 presented NY-ESO-1 fused to an anti-CD3 scFv. The ImmTAC™ construct comprises a single-chain Fv derived from anti-CD3 antibody UCHT-1 covalently linked to the C or N-terminus of the alpha or beta chain of the TCR. The NY-ESO-1 targeting reagent was shown to kill HLA-A2, antigen-positive tumour cell lines, and freshly isolated HLA-A2- and LAGE-1-positive NSCLC cells in-vitro and in the context of a mouse xenograft disease model. Additionally, Immatics™ has developed bispecific T cell-engaging receptors (TCER®) that are fusion proteins consisting of an affinity-maturated TCR and a humanized T cell-recruiting antibody with an effector function-silenced IgG1 Fc domain, Bunk, S., et. Al., Blood (2019) 134 (Supplement_1): 3368. Such bispecific molecules offer much needed therapeutic opportunities for treatment of cancer patients.

Cancer is the second leading cause of morbidity worldwide, accounting for nearly 1 in 6 of all deaths globally. Of the 8.8 million deaths caused by cancer in 2015, the cancers which claimed the most lives were from lung (1.69 million), liver (788,000), colorectal (774,000), stomach (754,000) and breast (571,000) carcinomas. The economic impact of cancer in 2010 was estimated to be USD1.16 trillion, and the number of new cases is expected to rise by approximately 70% over the next two decades (World Health Organisation Cancer Facts 2017). It is recognised that there is a need to provide better and safer ways of treating or eradicating cancer and immunotherapy using the body's natural defence systems is acknowledged to be safer than chemical intervention. Hence it is desirable to develop bispecific polypeptide molecules with structures that successfully permit engagement between the cancer antigen specificity of anti-cancer TCR binding domains with an antibody binding domain specificity to bind and recruit the body's own natural immune effector cells and activate them against a cancer or tumour target, making them advantageous candidates for cancer immunotherapy.

### SUMMARY OF THE INVENTION

According to the present invention, there is provided a bispecific polypeptide molecule capable of simultaneously binding a first and second antigen, wherein the bispecific polypeptide molecule comprises a first polypeptide chain and a second polypeptide chain, wherein the first polypeptide chain comprises: (i) a first antigen-binding domain, (ii) a first hinge domain subunit, (iii) a first Fc domain subunit; and the second polypeptide chain comprises: (i) a second antigen-binding domain, ii) a second hinge domain subunit, (iii) a second Fc domain subunit. According to the invention the first and second hinge domain subunits are capable of forming a stable association as a hinge domain and the first and second Fc domain subunits are capable of forming a stable association as an Fc domain or Fc domain portion such that the two polypeptide chains are connected by covalent and/or non-covalent bonds between the hinge domain subunits and Fc-domain subunits. In a preferred embodiment the first and second antigens are expressed on two distinct or different cells or target cells.

The invention further provides a polynucleotide encoding the bispecific polypeptide molecule of the invention and a vector for delivery of a polynucleotide to cells or host cells comprising a polynucleotide or vector according to the invention. Also provided is a host cell expressing the bispecific polypeptide molecule of the invention.

The invention further provides a process of producing the bispecific polypeptide molecule according to the invention, comprising the steps of a) culturing the host cell of the invention under conditions suitable for the expression of the bispecific polypeptide molecule and b) recovering the bispecific polypeptide molecule.

The invention further provides a pharmaceutical composition comprising the bispecific polypeptide, polynucleotide, vector or host cell according to the invention and a pharmaceutically acceptable carrier.

The invention further provides a method of treating cancer in a subject comprising administering a therapeutically effective amount of the bispecific polypeptide molecule, polynucleotide, vector, host cell or pharmaceutical composition according to the invention, to the subject.

The invention further provides a pharmaceutical composition comprising:
a) the bispecific polypeptide molecule, polynucleotide, vector, host cell or pharmaceutical composition according to the invention; and
b) an anti-cancer agent.

### BRIEF DESCRIPTION OF THE FIGURES

FIG. 1: Schematic diagram of TCE engager designs with short (SEQ ID NO.24), medium (SEQ ID NO.79), and long (SEQ ID NO.80) hinge domains. The scTv antigen binding domain is shown on the left, and the scFv antigen binding domain is shown on the right, large diamond shading represents the scTv V-beta domain and the scFv VH domains. The SEQ ID NOs specifying the scFv and scTv chains respectively for each of the three molecules are N010_009 (SEQ ID NO.29 & 30), N010_006 (SEQ ID NO.105 & 84), and N010_025 (SEQ ID NO.106 & 91). Distances between the two antigen-binding moieties is shown on the right of each of the three molecules for the sequence fragment at the junction between the chains of the scTv and the scFv domains. The Vα/TRAC sequence fragment is underlined, and the C-terminal residue of the scTv Vα domain is shown in lower case and the C-terminal residue of the scFv VH domain is shown in lower case italics. Horizontal arrows indicate distance between the C-terminal residues of the Vα domain and the VH domain which can be estimated as the sum of alpha-carbon to alpha-carbon distances (0.38 nm) plus the distance between the two alpha-carbons formed by the two alpha-carbons of the cys residues at the top of the hinge(0.6 nm), giving a reference value of 2.1 nm (4×0.38+1×0.6) for molecule N010_009, 5.9 nm (14×0.38+1×0.6) for molecule N010_006, and 9.7 nm (24×0.38+1×0.6) for molecule N010_025.

FIG. 2: Modeled structure of scTv antigen binding domain showing candidate linkers joining the V-alpha and V-beta domains (a) model placing chain order in Va-Vb direction (N-terminal to C-terminal), (b) or Vb-Va direction (N-terminal to C-terminal). Models created from PDB coordinate file 5e9d.

FIG. 3A: ELISA based IFN gamma release assays showing influence of hinge length on TCE bispecific polypeptide molecule activity in cell culture. The effect of varying the hinge length of TCE designs provided by molecules N010_009 (SEQ ID NO.29 & SEQ ID NO.30) short hinge, N010_006 (SEQ ID NO.105 & SEQ ID NO.84) medium hinge and N010_025 (SEQ ID NO.106 & SEQ ID NO.91) long hinge [see FIG. 1], tested using an IFNγ release assay where T2 cells were incubated with PRAME peptide, PBMCs plus bi-specific molecules ranging from 10fM to 100 nM. IFNγ measured at 48 hours.

FIG. 3B: ELISA based IFN gamma release assays showing influence of CPPC linker addition on TCE bispecific polypeptide molecule activity in cell culture. Stabilisation of the scTv of the TCE was investigated by varying the scTv domain of N010_009 (SEQ ID NO.29 & SEQ ID NO.30) by incorporation of a CPPC (molecule N010_010, SEQ ID NO.29 & SEQ ID NO.85) or CPPC+DDK staple (molecule N010_011, SEQ ID NO.29 & SEQ ID NO.86) using the T2 cellular assay described for FIG. 3A.

FIG. 4A: Incucyte cytotoxicity assay, Donor 1 PBMC effector cells & PRAME positive MEL624 cells and PRAME negative 92-1 cells as target cells, PRAME targeting TCR based bispecific polypeptide constructs N010_009 [SEQ ID No.29 & 30]comparator N011_003 [TCER SEQ ID NO.35 & 36], 1 pM to 10 nM.

FIG. 4B: Incucyte cytotoxicity assay, Donor 2 PBMC effector cells & PRAME positive MEL624 cells and PRAME negative 92-1 cells as target cells, PRAME targeting TCR based bispecific polypeptide constructs N010_009 [SEQ ID NO.29 & 30]comparator N011_003 [TCER SEQ ID NO.35 & 36], 1 pM to 10 nM.

### DEFINITIONS

Suitably, the polypeptides and polynucleotides used in the present invention are isolated. An “isolated” polypeptide or polynucleotide is one that is removed from its original environment. For example, a naturally occurring polypeptide or polynucleotide is isolated if it is separated from some or all of the coexisting materials in the natural system. A polynucleotide is considered to be isolated if, for example, it is cloned into a vector that is not a part of its natural environment.

“Naturally occurring” or “native”, which terms are interchangeable, when used with reference to a polypeptide or polynucleotide sequence means a sequence found in nature and not synthetically modified.

The term “artificial” when used with reference to a polypeptide or polynucleotide sequence means a sequence not found in nature which is, for example, a synthetic modification of a natural sequence, or contains an unnatural polypeptide or polynucleotide sequence or modified nucleotide or amino acid. The terms “engineer, engineered, or engineering”, include any manipulation of the peptide backbone or the post-translational modifications of a naturally occurring or recombinant polypeptide or fragment thereof.

Engineering additionally includes modifications of the amino acid sequence, of the glycosylation pattern, or of the side chain group of individual amino acids, as well as combinations of these approaches.

“Bispecific” or “bispecific polypeptide molecule” refers to an antigen binding molecule which is able to associate with at least two different antigens simultaneously by specifically interacting with or binding to at least two distinct epitopes or antigenic determinants. Preferably, the bispecific polypeptide molecule comprises two distinct antigen binding sites, each of which is specific for a different epitope or antigenic determinant for example which are independently expressed on two distinct cells, the antigenic determinant maybe expressed on the cell surface optionally in combination with a cell surface receptor, e.g. MHC or HLA or CD1 or antibody/immunoglobulin or B-cell receptor. Preferably the bispecific polypeptide molecule is an “antigen binding molecule” which is understood to mean a molecule that specifically binds an antigen or antigenic determinant or epitope. Examples of antigen binding molecules include, or include combinations of, T cell receptors or antibodies and derivatives, binding portions or binding fragments thereof, including the fragments thereof which retain the capacity to recognise or specifically bind an antigen or antigenic determinant of an antigen or epitope.

The term “antigen binding domain” refers to the part of an antigen binding molecule that comprises a structural amino acid sequence or area/region or set of such sequences or areas/regions which specifically binds to and/or is structurally complementary to part of or all of an antigen or antigenic determinant such that it may associate therewith. An antigen binding domain may be provided by, for example, one or more binding region of a variable domain of an antibody or TCR. The binding region of a variable domain may comprise an antibody light chain variable region (VL) optionally associated with the constant region CL or portion thereof and/or an antibody heavy chain variable region (VH) optionally associated with the constant region CH1 or antigen binding portion thereof. For example, the binding region of a variable domain may comprise an antibody light chain variable region (VL) and/or an antibody heavy chain variable region (VH), or portion thereof which retains binding and specificity to the antigen. An antigen binding domain may likewise be provided by one or more binding region of a variable domain of a TCR. For example, the binding region of a variable domain may comprise a TCR alpha chain variable region (Vα) optionally associated with the constant region Cα or portion thereof and/or a TCR beta chain variable region (Vβ) optionally associated with the constant region Cβ or portion thereof. For example, the binding region of a variable domain may comprise a TCR alpha chain variable region (Vα) and/or a TCR beta chain variable region (Vβ) or portion thereof which retains binding and specificity to the antigen or epitope.

The terms “first” and “second” with respect to antigen-binding domain, binding region of a variable domain, hinge domain subunit, Fc domain subunit, are used to distinguish when there is more than one of each type of moiety and is not intended to confer a specific order or orientation of the bispecific polypeptide molecule unless explicitly so stated.

The term “antibody” includes but is not limited to monoclonal antibodies, polyclonal antibodies or recombinantly produced antibodies which may be human, humanised antibodies, and derivatives thereof, including the fragments thereof which retain the capacity to recognise or specifically bind an antigen or antigenic determinant of an antigen or epitope.

An “antibody fragment” or means a molecule that comprises a portion of an intact antibody that binds the antigen to which the intact antibody binds. Examples of antibody fragments include but are not limited to Fv, Fab, Fab′, Fab′-SH, F(ab′)₂, diabodies, for example with two antigen-binding sites that may be bivalent or bispecific, linear antibodies, single-chain antibody molecules such as scFv molecules, and single-domain antibodies. Single-domain antibodies, which may be human single-domain antibodies, are antibody fragments comprising all or a part of the variable domain of an antibody, i.e., either heavy chain variable domain or light chain variable domain.

With respect to an antibody or antibody fragment, the term “variable region” or “variable domain” refers to the domain of an antibody heavy or light chain that is involved in binding the antibody to antigen, for example the variable domains of the heavy chain (VH) and light chain (VL). Each variable domain comprises four conserved framework regions (FR) and three hypervariable regions (HVRs). A single VH or VL domain may be sufficient to confer antigen-binding specificity. The “hypervariable region” (HVR), as used with respect to an antibody or antibody fragment, refers to each of the regions of an antibody variable domain which are hypervariable in amino acid sequence and comprise a complementarity determining region (CDR) of high variability and adjacent variable residues of the framework, these regions are involved in antigen recognition. Antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. CDRs generally comprise the amino acid residues that form the hypervariable regions. Hence hypervariable regions (HVRs) are also referred to as “complementarity determining regions” (CDRs), and these terms are used herein interchangeably in reference to portions of antibody variable region forming “antigen binding regions”. Those skilled in the art can routinely determine which residues comprise a particular CDR given the variable region amino acid sequence of the antibody.

Humanised antibodies are antibodies (or parts thereof) from non-human species whose protein sequences have been modified to increase their similarity to antibodies or antibody variants produced naturally in humans. The process of “humanization” is usually applied to monoclonal antibodies developed for administration to humans (for example, antibodies developed as anti-cancer drugs). Suitable methods for humanization are known from the literature.

An “antigen binding site” refers to the structural polypeptide surface or site situated on an antigen binding molecule which provides interaction with the antigen. One or more binding regions or antigen binding regions of variable domains of the antigen binding molecule may contribute to the antigen binding site. For example, the antigen binding site of an antibody or a TCR comprises amino acid residues from one or more of the complementarity determining regions (CDRs) which may be brought together to provide a surface which interacts with an antigen or antigenic determinant. For example, a Fab, a scFv molecule or scTv molecule typically have a single antigen binding site, usually comprising one or more CDRs.

An “antigen” refers to a molecule that can bind to or be recognised by an antigen binding molecule or antigen binding domain of an antigen binding molecule, such as an antibody or T-cell receptor and on binding may trigger an immune response. Antigens can be proteins, peptides, polysaccharides, lipids, or nucleic acids for example cell surface expressed proteins, peptides, polysaccharides, lipids, or nucleic acids, as an example the antigen may be a cell surface receptor. A “target cell antigen” as used herein refers to an antigen or an antigenic determinant or epitope presented on the surface of a target cell, for example which is bound by an antigen binding domain or antigen binding site, for example an antigen presented on the surface of a tumour cell or a cancer cell or a cell of the tumour stroma or antigen on the surface of an immune effector cell. The immune effector cell and tumour cell or a cancer cell may be understood to be a “target cell” in this context.

A “T-cell receptor” (TCR) refers to a heterodimeric protein consisting of the highly variable alpha (α) and beta (β) chains and which recognises and binds to HLA-bound antigenic peptide, pMHC. The α and β chains are composed of extracellular domains comprising a constant (C) region and a variable (V) region. A TCR can exist as a disulphide-linked membrane-anchored heterodimeric protein comprising the extracellular variable domain as well as, at its C terminus, transmembrane and intracellular domains and can associate with the invariant CD3 chain molecules to form a complete functioning TCR. In the cell presented TCR, the Constant region is proximal to the cell membrane, followed by a transmembrane region and a short cytoplasmic tail, while the Variable region binds to the ligand. T-cells expressing this receptor are referred to as α:β (or αβ) T-cells. An immature form of an entire TCR also comprises a leader peptide sequence at its N terminus which is removed after translation by cellular peptidases such as signal peptidase. Each chain has a connecting peptide region which links the transmembrane and intracellular regions to the extracellular domain at its C terminus. The soluble form of the TCR consists of the extracellular domain (also called the TCR antigen-binding domain). The extracellular region of each chain comprises three CDRs (CDR1, CDR2, CDR3) and four framework regions which are either side of the CDRs, and a constant region. The TCR variable domain is formed of variable regions of both the TCR α-chain and β-chain each of which has three hypervariable regions called complementarity determining regions (CDRs). There is also an additional area of variability on the β-chain (HV4) that does not normally contact antigen and, therefore, is not considered a CDR. In general, the antigen-binding site is formed by the CDR loops of the TCR α-chain and β-chain. CDR1α and CDR2α are encoded by the individual Vα genes whereas CDR1β and CDR2β are encoded by the individual Vβ genes. The CDR3 of the TCR α-chain is especially hypervariable due to the potential for nucleotide addition and removal around the joining of the variable (V) region and a joining (J) region. The TCR β-chain CDR3 has even more capacity for variation as it can also include a diversity (D) gene. CDR3 is the main CDR responsible for recognizing HLA-bound antigenic peptides (the usual target for αβ TCRs). However, in some cases CDR1 of the a chain has also been shown to interact with the N-terminal part of the HLA-bound antigenic peptide, and CDR1 of the β-chain may interact with the C-terminal part of the HLA-bound antigenic peptide. An antigen binding domain of the bispecific polypeptide molecule of the invention can be an antigen binding fragment of a TCR and can comprise or consist of a TCR extracellular domain (also called the antigen-binding domain), e.g. a soluble form of the TCR, wherein the TCR comprises a TCR alpha chain variable domain (Vα) and a TCR beta chain variable domain (Vβ). Alternatively, it can comprise of a single-chain format in which TCR variable domains are connected by a flexible peptide linker, so called single chain TCRs (scTv) or may comprise of a single-variable-domain TCR (svd TCR) that utilizes only the variable domain of the β chain (Vβ) or alpha chain (Vα), preferably (Vβ). Such an antigen binding fragment of a TCR retains the function of cancer-specific binding i.e. to bind to its target antigen or epitope, for example to the binding motif or epitope for example presented in complex with the MHC (pMHC) presented on the surface of the cancer or tumour cell.

The term “domain”, when used with reference to a TCR, is generally used to refer to a part of the TCR formed of the corresponding region of the two chains. For example, the transmembrane regions of the α and β chains form the transmembrane domain, etc. The term “intracellular” domain or region is used interchangeably with the term “cytoplasmic” domain or region and in the literature, this is sometimes referred to as the “cytosolic” domain or region. The domain may also be an antigen binding domain as described above with respect to TCRs.

The term “epitope” or “antigenic determinant” refers to a site or contiguous stretch of amino acids or a conformational configuration made up of different regions of non-contiguous amino acids on a polypeptide macromolecule to which an antigen binding site or antigen binding region of an antigen binding domain binds, forming an antigen binding site-antigen complex.

“Polypeptide” refers to a molecule comprising amino acid monomers linearly linked by peptide bonds. Examples include (a) a single-chain Fv (scFv) molecule, i.e. wherein the light chain variable region and the heavy chain variable region of an immunoglobulin molecule are connected by a peptide linker to form a single peptide chain, (b) a single-chain Tv (scTv) molecule, i.e. wherein the alpha chain variable region and the beta chain variable region of a TCR molecule are connected by a peptide linker to form a single peptide chain.

The term “fused” is synonymous with the term “linked”, meaning linked or attached directly to such that the fused components follow adjacent to each other, generally the linkage is by covalent bonding such as a peptide bond, for example a TCR antigen binding domain or an scTv fragment and a hinge domain subunit are linked by peptide bonds, either directly or via one or more peptide linkers.

The “hinge” or “hinge region” or “hinge domain” refers to the flexible portion of a heavy chain located between the CH1 domain and the CH2 domain in an antibody. It is approximately 25 amino acids long, and is divided into an “upper hinge,” a “middle hinge” or “core hinge,” and a “lower hinge.” A “hinge subdomain” refers to the upper hinge, or middle (or core) hinge or the lower hinge. The core hinge region usually contains at least one cysteine-bridge connecting the two heavy chains. Furthermore, mutations can be made in the lower hinge region to remove antibody-dependent cell-mediated cytotoxicity effector functions. A hinge domain connects the antigen binding domains to the Fc domain in the bispecific polypeptide molecule herein. The amino acids sequences of the hinges of an IgG1, IgG2, and IgG4 molecule are (EU numbering indicated):

IgG1 Hinge:
(SEQ ID NO. 20)
EPKSCDKTHTCPPCPAPELLG
IgG2 Hinge:
(SEQ ID NO. 21)
ERKCCVECPPCPAPPVAGP
IgG4 Hinge:
(SEQ ID NO. 22)
ESKYGPPCPSCPAPEFLG

“Fc domain” or “Fc region” means a C-terminal region of an immunoglobulin heavy chain that contains at least a portion of the constant region, i.e., the dimeric heavy chain region comprising a pair of constant heavy chains 2 and 3, e.g. a pair of CH2CH3 chains, e.g. (CH2CH3) chains, or a portion of said pair for example truncated in CH2 and/or CH3 sequence. “Fc domain subunit” denotes the monomeric domain or region of the Fc domain. The fragment crystallizable region (Fc region) or portion thereof is preferably selected from IgG1, IgG2 and IgG4, or a recombinant variant thereof for example comprising amino acid substitutions, deletions, or additions which may be derived from an alternative IgG subclass, or portion thereof (optionally human) or may be a recombinant chimera of such domains, for example which is derived from two or more human immunoglobulin heavy chains, for example two or more human immunoglobulin heavy chain CH2 domains, which human immunoglobulins are selected from IgG1, IgG2 and IgG4. An Fc region may comprise mutations shown to increase the binding affinity to neonatal Fc receptor (FcRn) to extend the half-life in vivo or the serum half-life of the Fc-containing molecule. Additionally, the Fc domain can comprise a CH2 comprising at least one effector function silencing mutation. The human IgG heavy chain Fc region is usually defined to extend from Cys226, or from Pro230, to the carboxyl-terminus of the heavy chain, the C-terminal Lys447 may or may not be present. Unless otherwise specified herein, the numbering of amino acid residues in the Fc region is the EU numbering system.

An “Fc domain subunit” or “subunit” of an Fc domain refers to one of the two polypeptides forming the dimeric Fc domain, e.g., (CH2CH3) chains, or a portion thereof for example truncated in CH2 and/or CH3 sequence., i.e., an Fc domain subunit can be a polypeptide sequence comprising the C-terminal constant regions of an immunoglobulin heavy chain or portion thereof. Two such Fc domain subunits are capable of stable self-association to form an Fc domain or Fc region. For example, the IgG Fc domain subunit comprises a monomeric IgG CH2 and an IgG CH3 constant domain, optionally CH3 truncated.

The term “effector function” refers to antibody Fc region associated biological activities. Examples of antibody effector functions include: C1q binding and complement dependent cytotoxicity (CDC), Fc receptor binding, antibody-dependent cell-mediated cytotoxicity (ADCC), antibody-dependent cellular phagocytosis, cytokine secretion, immune complex-mediated antigen uptake by antigen presenting cells, down regulation of cell surface receptors, for example the B cell receptor, and B cell activation.

The “class” of an antibody or immunoglobulin refers to the isotype of constant region of its heavy chain which may be selected from α, δ, ε, γ, and μ, to give rise to the five major classes of antibodies: IgA, IgD, IgE, IgG, and IgM. Isotype subclasses are also known e.g., IgG1, IgG2, IgG3, IgG4, IgA1, and IgA2.

“Framework” or “FR” with respect to antibody or TCR sequences or antibody or TCR derived sequences, e.g. of an antigen binding domain, refers to variable domain residues other than hypervariable region (HVR) residues or complementarity-determining region (CDR) residues. The FR of a variable domain generally consists of four FR domains: FR1, FR2, FR3, and FR4. Accordingly, the HVR/CDR and FR sequences generally appear in the following sequence in: FR1-HVR1-FR2-HVR2-FR3-HVR3-FR4 or FR1-CDR1-FR2-CDR2-FR3-CDR3-FR4, i.e., in antibody VH or VL or in TCR Vα or Vβ.

A “pharmaceutical composition” refers to a formulation or preparation of an active ingredient which maintains the biological activity of the active ingredient, e.g., bispecific polypeptide molecule, as effective for administration to a subject.

A “pharmaceutically acceptable carrier” refers to a pharmaceutically neutral component in a pharmaceutical composition, which is suitable for the stabilisation and/or conveyance of the active ingredient, e.g., bispecific polypeptide molecule, for administration to a subject. A pharmaceutically acceptable carrier includes, but is not limited to, a buffer, excipient, stabilizer, or preservative.

A “subject” is a mammal, particularly a human individual.

A “therapeutically effective amount” or “effective amount” of an agent, e.g. a pharmaceutical composition, refers to an amount effective, at dosages and for periods of time necessary, to achieve the desired therapeutic or prophylactic result, and/or for example eliminates, decreases, delays, minimizes or prevents the effects or magnitude of a disease, for example cancer or tumour, as judged by a recognised clinical endpoint.

As used herein, the term “bind”, “specific binding” or “specifically binds” or “specifically binding” in relation to the binding of A to B means that A binds to B, for example at or within a respective specific binding site, domain or pocket, with an affinity typically associated with the binding of ligands to receptors or typically associated with molecules of the immune system, such as antibodies and T-cell receptors, optionally a binding affinity level of micromolar or nanomolar affinity or greater, such that the affinity of binding of A to B exceeds or greatly exceeds that of the binding of A to other molecules not intended to be targeted by A. Binding is “selective” where the antigen and can be discriminated from unwanted or non-specific interactions. The ability to bind to a specific antigenic determinant or epitope can be measured either through an enzyme-linked immunosorbent assay (ELISA) or other techniques familiar to one of skill in the art, e.g. surface plasmon resonance technique and traditional binding assays. Preferably the extent of binding to an unrelated protein is less than about 10% of the binding to the antigen as measured by a suitable technique and preferably with a dissociation constant (K_{D}) of <1 μM, <100 nM, <10 nM, <1 nM, <0.1 nM, <0.01 nM, or <0.001 nM (e.g. 10~⁸M or less, e.g., from 10^{~8}M to 10^{~13}M, e.g., from 10^{~9}M to 10^{~13}M). The term “being specifically bound” is to be interpreted in a similar sense.

“Affinity” refers to the magnitude of the sum-total of non-covalent interactions between a single binding site of a binding molecule (e.g., antigen binding domain for example, a scTv, scFv, a receptor) and its binding partner (e.g., a ligand, antigen, antigenic determinant), hence the “binding affinity” refers to intrinsic binding affinity arising through a 1:1 interaction between members of such a binding pair. The affinity of a molecule A for its partner B can be denoted by the dissociation constant (KD), which is the ratio of dissociation and association rate constants (K-off and K-on, respectively). Affinity can be measured by methods known in the art, for example Surface Plasmon Resonance (SPR).

As used herein, the term “specifically reactive” when used in relation to the binding of A to B means that when A specifically binds to target B, a biological activation is initiated either in B or if B is a receptor is transduced through B to produce a biological activation effect to an extent that greatly exceeds that activation when the binding of A to other targets not intended to be targeted by A or not specifically bound by A, or that activation in B exceeds that in absence of the specific binding of A. By way of example (and with reference to the bispecific polypeptide molecule according the present invention) an antigen-binding domain, such as an antibody derived antigen binding domain, like an scFv, which recognises and specifically binds to a cell surface antigen expressed on the surface of an immune effector cell, such as an activating receptor on the surface of the immune effector cell like a T-cell (e.g., CD3), may by its specific binding induce activation of the immune effector cell, e.g. transduced through such bound receptor. The context of “specifically reactive” implies that activation is induced in the immune cell to a higher level than the activation measured in the absence of the binding event.

In the context of the bispecific polypeptide molecule according to the present invention, the TCR derived antigen binding domain or antigen binding site “specifically binds” an MHC-associated peptide epitope of a tumour or cancer polypeptide antigen presented at the cell surface of a tumour cell or a cancer cell. The TCR derived antigen binding domain or antigen binding site may also be “specifically reactive” to said tumour cell or cancer cell and/or tumour cell or cancer cell presented cancer polypeptide antigen or antigenic peptide fragments thereof, in the context of peptide MHC presentation. This may be discovered by determining if upon interaction with the cancer or tumour polypeptide antigen or antigenic peptide fragment presented by the tumour or cancer cell, there is an induced activation of an immune response to said tumour cell or cancer cell, for example an induced activation of an immune effector cell bound by the antibody derived antigen binding domain of the bispecific polypeptide molecule, to a higher level than the activation measured in the absence of the bound tumour or cancer cell. Comparison may be made for example to activation measured when binding a non-cancer peptide antigen MHC presented at the cell surface of a non-tumour cell or non-cancer cell or activation measured when binding a non-target cancer MHC presented peptide antigen which is not bound or recognised by the subject TCR or TCR derived antigen binding domain or antigen binding site. Immune cell or T cell activation can be evaluated with any of the following measurements: cytokine release (IFN gamma or TNF alpha), chemokine release, immune cell proliferation, immune cell expression of activation markers, immune cell target cell killing, induction of transcription factors or of reporter genes of the immune cell.

The term “immune effector cell” The term “immune effector cell” refer to cells of the lymphoid lineage including T cells, Natural Killer T (NKT) cells, and precursors thereof including embryonic stem cells, and pluripotent stem cells from which lymphoid cells may be differentiated. Preferably the immune cells and immune cell clones are T cells which can for example include, but are not limited to, helper T cells, cytotoxic T cells, memory T cells (including central memory T cells, stem-cell-like memory T cells or stem-like memory T cells), and effector memory T cells such as TEM cells and TEMRA cells, Regulatory T cells or suppressor T cells, Natural killer T cells, Mucosal associated invariant T cells, TILs (tumour infiltrating lymphocytes) and gamma-delta T cells. Preferably, the immune cells and immune cell clones are T cells optionally CD4⁺ T cells or a CD8⁺ T cells. Accordingly, the immune cells and immune cell clones may be T-cells, optionally CD4+ T cells or CD8+ T cells, or the immune cells and immune cell clones may be a population of T-cells, optionally CD4+ T cells; or CD8+ T cells, or a mixed population of CD4+ T cells and CD8+ T cells. Suitably the immune effector cell is a T-cell preferably a CD8+ T-cell.

The term “reduced binding”, means reduction in binding affinity or avidity for the respective interaction conversely, “increased binding” refers to an increase in binding affinity or avidity for the respective interaction.

The term “activating receptor” refers to an antigenic determinant or epitope, for example receptor molecule, such as for example CD3, expressed on the surface of a T lymphocyte, particularly a CD8+ or cytotoxic T lymphocyte, which is capable of inducing T cell activation upon interaction with an antigen binding molecule, the binding of which may induce T cell activation by triggering the signalling cascade through the T cell receptor complex. In a particular embodiment the activating receptor is the activating T cell antigen CD3E.

The term “immune effector cell activation” refers to one or more cellular response of an immune effector cell for example a T lymphocyte where such activation is also considered “T cell activation”, for example a cytotoxic T lymphocyte activation, e.g. selected from: proliferation, differentiation, cytokine secretion, cytotoxic effector molecule release, cytotoxic activity, and expression of activation markers. The immune effector cell/T cell activating bispecific antigen binding molecules of the invention are capable of inducing immune effector cell/T cell activation. Suitable assays to measure immune effector cell/T cell activation are known in the art. For example, immune effector cell activation can be evaluated with any of the following measurements: cytokine release (IFN gamma or TNF alpha), chemokine release, immune cell proliferation, immune cell expression of activation markers, immune cell target cell killing, induction of transcription factors or of reporter genes of the immune cell.

A “mutation” or “mutations”, for example in the Fc domain or domain subunit or portion thereof, which facilitates the formation of heterodimers is a mutation which is understood to promote the association of the first and the second subunit of the Fc domain subunits. Such association reduces or prevents the association of a polypeptide comprising an Fc domain subunit with a polypeptide comprising the same Fc domain subunit to form a homodimer of polypeptide chains in the bispecific polypeptide molecule. Mutations promoting heterodimer association as used herein particularly includes separate modifications made to each of the two Fc domain subunits desired to associate (i.e. the first and the second Fc domain subunits), wherein the modifications are complementary to each other so as to promote association of the two Fc domain subunits. For example, a modification promoting association may alter the structure or charge of one or both of the Fc domain subunits so as to make their association sterically or electrostatically favourable, respectively. Such dimerisation or heterodimerisation may then occur between a polypeptide comprising the first Fc domain subunit and a polypeptide comprising the second Fc domain subunit, which may be non-identical in the sense that further components fused to each of the subunits, e.g., antigen binding domains are not the same.

In some embodiments the modification promoting association comprises an amino acid mutation in the Fc domain, specifically an amino acid substitution. In a particular embodiment, the modification promoting association comprises separate amino acid mutations, specifically amino acid substitutions, in each of the two subunits of the Fc domain.

“Naturally occurring” or “native”, which terms are interchangeable, when used with reference to a polypeptide or polynucleotide sequence means a sequence found in nature and not synthetically modified.

The term “artificial” when used with reference to a polypeptide or polynucleotide sequence means a sequence not found in nature which is, for example, a synthetic modification of a natural sequence, or contains an unnatural polypeptide or polynucleotide sequence.

The term “engineered” or “recombinant” when used with reference to a cell means a cell not found in nature which is, for example, a synthetic modification of a natural cell, for example, because it contains or expresses foreign elements and/or lacks natural elements.

The term “heterologous” when used with reference to the relationship of one polynucleotide or polypeptide to another polynucleotide or polypeptide indicates that the two or more sequences are not found in the same relationship to each other in nature.

The term “heterologous” when used with reference to the relationship of one polynucleotide or polypeptide sequence to a cell means a sequence which is not isolated from, derived from, expressed by said cell or associated with or based upon a naturally occurring polynucleotide or polypeptide sequence found in the said cell.

The term “vector” or “expression vector” or “expression construct” refers to a DNA molecule that is used to introduce and direct the expression of a specific nucleic acid in a host cell to which it is operably associated. Once the expression vector is inside the host cell, the ribonucleic acid molecule or protein that is encoded by the nucleic acid is produced by the cellular transcription and/or translation machinery. In one embodiment, the expression vector of the invention comprises a nucleic acid or polynucleotide sequences that encodes the bispecific polypeptide molecule of the invention or fragments or portions thereof.

The term “host cell” refers to a cell into which exogenous nucleic acid has been introduced, including the progeny of such cell. Host cells include “transformants” and “transformed cells,” which include the primary transformed cell and progeny derived from such cells. Examples of suitable mammalian host cells include cells such as CHO cells, BHK cells, NSO cells, SP2/0 cells, YO myeloma cells, P3X63 mouse myeloma cells, PER cells, PER.C6 cells or hybridoma cells, and cells comprised within a transgenic animal or animal tissue, yeast cells or bacterial cells may also serve as host cells, for example E. coli, Bacillus subtilis, Saccharomyces cerevisiae or Aspergillus.

“Activating Fc receptor”, means an Fc receptor that following engagement by an Fc domain of an antibody elicits signalling events that stimulates the receptor-bearing cell to perform effector functions. Human activating Fc receptors include: CD16a, CD32, CD64, CD89.

“Effective amount” of an agent refers to the amount that is necessary to result in a physiological change in the cell or tissue to which it is administered or measurably treats (ameliorates/improves) a disease or condition.

“Treatment”, “treat” or “treating” refers to clinical intervention in an attempt to alter the natural course of a disease in the individual being treated, and includes preventing, alleviating or diminishing of any direct or indirect pathological or symptomatic consequences of the disease or condition, optionally where said disease or condition is cancer.

### DETAILED DESCRIPTION

According to a first aspect of the present invention there is provided a bispecific polypeptide molecule capable of simultaneously binding a first and second antigen, wherein the bispecific polypeptide molecule comprises a first polypeptide chain and a second polypeptide chain, wherein:
(A) the first polypeptide chain comprises: (i) a first antigen-binding domain, (ii) a first hinge domain subunit, (iii) a first Fc domain subunit; and
(B) the second polypeptide chain comprises: (i) a second antigen-binding domain, ii) a second hinge domain subunit, (iii) a second Fc domain subunit;
wherein the first and second hinge domain subunits are capable of forming a stable association as a hinge domain and the first and second Fc domain subunits are capable of forming a stable association as an Fc domain or Fc domain portion such that the two polypeptide chains are connected by covalent and/or non-covalent bonds between the hinge domain subunits and Fc-domain subunits, preferably wherein the a first and second antigens are different antigens and are expressed on two different and/or distinct cells, for example a first target cell and a second target cell respectively.

Preferably the bispecific polypeptide molecule of the invention may simultaneously bind a first antigen and second antigen respectively. The first antigen may be target cell antigen, preferably wherein the target cell is an immune effector cell, preferably said antigen is activating receptor on an immune effector cell, for example CD3. The second antigen may be a target cell antigen, preferably wherein said target cell is a cancer or tumour cell or cell presenting (on its surface, optionally in complex with a receptor, optionally MHC or HLA) a cancer or tumour cell antigen, preferably wherein the target cell antigen is a tumour cell or cancer cell antigen. Preferably said simultaneous binding provides immune effector cell activation and/or provides immune effector cell target cell killing wherein said target cell is a cell presenting a cancer or tumour cell antigen, for example a cancer or tumour cell. Preferably said simultaneous binding of each antigen is high affinity binding and/or specifically reactive binding.

Preferably the first polypeptide chain comprises: (i) a first antigen-binding domain which is directly linked to or fused to (ii) a first hinge domain subunit, which is directly linked to or fused to (iii) a first Fc domain subunit; preferably in an N-terminal to C-terminal direction.

Preferably the second polypeptide chain comprises: (i) a second antigen-binding domain which is directly linked to or fused to (ii) a second hinge domain subunit, which is directly linked to or fused to (iii) a second Fc domain subunit; preferably in an N-terminal to C-terminal direction.

### The First Antigen-Binding Domain

According to the invention the first antigen binding domain may be derived from one or more antibody, which is preferably a human or humanised antibody, preferably which is capable of binding or specifically binding to and/or recruiting immune effector cells, preferably human immune effector cells, for example by specifically binding to an antigen of said effector cells or expressed on the surface of said effector cells, i.e. a surface antigen. Preferably said antigen is activating receptor on an immune effector cell, for example CD3Preferably said binding is specifically reactive to an immune effector cell and is capable of achieving immune effector cell activation.

Preferably the first antigen-binding domain comprises a first binding region of a variable domain (VD1) of an antibody and a second binding region of a variable domain (VD2) of an antibody, preferably wherein the first binding region (VD1) and the second binding region (VD2) associate to form a first antigen binding site (VD1)(VD2). In a preferred embodiment said VD1 and VD2 are connected to each other by a first linker (LNK1) connecting said domains wherein the first binding region (VD1) and the second binding region (VD2) associate to form a first antigen binding site (VD1)(VD2).

Accordingly, the first antigen-binding domain may comprise a first binding region of a variable domain (VD1) of an antibody and a second binding region of a variable domain (VD2) of an antibody and a first linker (LNK1) connecting said domains preferably wherein the first binding region (VD1) and the second binding region (VD2) associate to form a second antigen binding site (VD1)(VD2). The first linker LNK1 may be connected to or fused to VD1 and/or VD2 by a peptide bond, the connection may be to the C-terminal or N-terminal of said VD1 or VD2 depending on the chain orientation of the variable domains. Where VD1 is at the N-terminal then LK1 is connected to the C-terminal of VD1 and the N-terminal of VD2. Where VD2 is at the N-terminal then LK1 is connected to the C-terminal of VD2 and the N-terminal of VD1. In some embodiments LNK1 may be connected to, or peptide bonded to the VL or VH domain or antigen or epitope binding portion thereof, in some embodiments LNK1 may be connected to or peptide bonded to amino acid residues derived from a CL region or CH1 region respectively linked to the c-terminus of the respective VL or VH, for example as already described herein.

The order of the binding regions of the first antigen-binding domain can be VD1 N-terminal to VD2 or VD2 N terminal to VD1, preferably VD1 and VD2 are connected by a linker molecule or linker LNK1, for example from N terminus to C terminus the orientation may be either VD1- LNK1-VD2 or VD2- LNK1-VD1, such that the first binding region (VD1) and the second binding region (VD2) associate to form the first antigen binding site (VD1)(VD2), i.e. connected by LNK1. Preferably the order of the binding regions of the first antigen-binding domain is VD1 N-terminal to VD2 or N-terminal VD1-LNK1-VD2 where VD1 and VD2 are connected by the linker LNK1.

Preferably the first binding region of a variable domain (VD1) and/or second binding region of a variable domain (VD2) are derived from one or more antibody which is preferably a human or humanised antibody, which is capable of binding or specifically binding to and/or recruiting immune effector cells, preferably human immune effector cells, for example by specifically binding to an antigen of said effector cells or expressed on the surface of said effector cells, i.e. a surface antigen, optionally an activating receptor. Preferably said binding is specifically reactive for an immune effector cell and is capable of achieving immune effector cell activation. Preferably said first binding region of a variable domain (VD1) and/or second binding region of a variable domain (VD2) and/or first antigen binding site (VD1)(VD2), have the foregoing binding, binding specificity and activation functions and are capable of achieving immune effector cell activation. The antibody from which the binding region and/or domains are derived is preferably of the class IgG, for example any of IgG1, IgG2, IgG3, IgG4 or recombinant chimera thereof.

The first binding region of a variable domain (VD1) may comprise an antibody variable light domain (VL) or epitope binding portion thereof, optionally it may further comprise part or all of an antibody light chain constant domain, CL, preferably linked to or fused to the C-terminus of the VL domain. Alternatively, VD1 does not comprise an antibody light chain constant domain, CL. Preferably VD1 comprises or consists of an antibody variable light domain (VL) or epitope binding portion thereof. The second binding region of a variable domain (VD2) may comprise an antibody variable heavy domain (VH) or epitope binding portion thereof, optionally it may further comprise part or all of an antibody heavy chain constant domain 1, CH1, preferably linked to or fused to the C-terminus of the VH domain. Alternatively, VD2 does not comprise an antibody heavy chain constant domain, CH1. Preferably VD2 comprises or consists of an antibody variable heavy domain (VH) or epitope binding portion thereof.

Preferably (a) the first binding region of a variable domain (VD1) may comprise an antibody variable light domain (VL) or epitope binding portion thereof, which comprises any of one, two or three light chain CDR sequences, preferably three light chain CDR sequences, and any of one, two, three or four light chain framework sequences, preferably four light chain framework sequences, and/or (b) the second binding region of a variable domain (VD2) may comprise an antibody variable heavy domain (VH) or epitope binding portion thereof, which comprises any of one, two or three heavy chain CDR sequences, preferably three heavy chain CDR sequences, and any of one, two, three or four heavy chain framework sequences, preferably four heavy chain framework sequences.

According to the present invention the first antigen-binding domain may comprise a first binding region of a variable domain (VD1) of an antibody and a second binding region of a variable domain (VD2) of an antibody and a first linker (LNK1) as hereinabove described. Preferably the introduction of said linker LNK1 provides improved stability of the (VD1)(VD2) binding site and/or improved binding specificity and/or affinity of the binding site for the respective antigen or epitope or target cell for example as measured by methods disclosed herein. Accordingly, the first antigen-binding domain may comprise or consist of a Fab or epitope binding portion thereof or a single chain Fv (scFv) or epitope binding portion thereof, preferably an scFv.

Preferably the linker LNK1 is a flexible linker permitting the connected domains a degree of movement and/or interaction and to achieve appropriate functional separation of the domains, preferably composed of small, non-polar (e.g. glycine) and/or small polar (e.g. serine or threonine) amino acids. Preferably LNK1 is of between 3 and 20 amino acids in length, i.e. any of 3, 4, 5, 6, 7, 8, 9, 10, 11, 12, 13, 14, 15, 16, 17, 18, 19 or 20 amino acids, optionally comprising glycine and serine residues, or glycine and threonine residues. A suitable example of a Gly and Ser containing flexible linker is GSAGSAAGSGEF (SEQ ID NO.37). The linker may also comprise Ala to maintain flexibility, as well as polar amino acids such as Lys and Glu to improve solubility. Preferably the linker is a “GS” linker, i.e. having sequences consisting primarily of stretches of glycine and serine residues, optionally of the motif or sequence (Gly-Gly-Gly-Ser)ₙ (Gly-Gly-Gly-Gly-Ser)ₙ, preferably where the copy number “n”, is any one of 1, 2, 3 or 4, more preferred is either (Gly-Gly-Gly-Ser)₃ or (Gly-Gly-Gly-Ser)₄. In other embodiments the linker may be selected from the sequences KESGSVSSEQLAQFRSLD (SEQ ID NO.38), EGKSSGSGSESKST (SEQ ID NO.39), (Gly)₆ and (Gly)₈. Preferably the linker is any of GGGSGGGG (SEQ ID NO.40), GGGS (SEQ ID NO.41), GGGGS (SEQ ID NO.42), TVLRT (SEQ ID NO.43), TVSSAS (SEQ ID NO.44), and TVLSSAS (SEQ ID NO.45), most preferably the linker is GGGGSGGGGSGGGGSGGGGS (SEQ ID NO.46, G4S4 linker) or GSADDAKKDAAKKDGKS (SEQ ID NO.47, GS2 or Kranz linker).

According to one embodiment of the invention VD1 and VD2 may comprise an engineered disulphide bridge capable of introducing a covalent bond between VD1 and VD2, preferably wherein one or more cysteines are recombinantly introduced into a framework region of VD1 and/or VD2, preferably wherein said cysteines are recombinantly introduced into a framework region, preferably a surface exposed framework region, for example into framework region (FR) 4 in case of VL and framework region (FR)2 in case of VH or in the alternative are recombinantly introduced into framework region (FR) 4 in case of VH and framework region (FR) 2 in case of VL. For example where the chain order from N-terminus to C-terminus is VL-VH said cysteines may be recombinantly introduced into for example into framework region (FR) 2 in case of VL and framework region (FR)4 in case of VH, or where the chain order from N-terminus to C-terminus is VH-VL said cysteines may be recombinantly introduced into for example into framework region (FR) 2 in case of VH and framework region (FR)4 in case of VL. According to these embodiments the linker LNK1 may comprise one or more cysteine (Cys) residues, preferably two cysteine residues, preferably LNK1 comprises a first LNK1 Cys and a second LNK1 Cys. Said cysteine residues may be native to the sequence, for example a structurally conserved Cys, or may be recombinantly introduced not said sequence. For example, said linker LNK1 may comprise the sequence CPPC (SEQ ID NO:52). Accordingly, a first disulphide bond may be formed between a recombinantly introduced VH cysteine (Cys) and a first LNK1 Cys or a second disulphide bond may be formed between a recombinantly introduced VL Cys and a second LNK1 Cys; or a first disulphide bond may be formed between a recombinantly introduced VH cysteine (Cys) and a first LNK1 Cys and a second disulphide bond may be formed between a recombinantly introduced VL Cys and a second LNK1 Cys. In the alternative the VH Cys and/or VL Cys may be native to the sequence, for example a structurally conserved Cys, preferably a structurally conserved surface exposed VH Cys and/or VL Cys. Preferably the introduction of said disulphide bridge provides improved stability of the (VD1)(VD2) binding site and/or improved binding specificity and/or affinity of the binding site for the respective antigen or epitope or target cell, for example as measured by methods disclosed herein.

Preferably, the LNK1 may comprise a contiguous amino acid sequence derived from an immunoglobulin (Ig) hinge region, optionally the Ig hinge region is derived from (i.e. comprise all or part of the sequence thereof) a human or a non-human Ig hinge region, preferably human Ig hinge region, further preferably the human Ig hinge region is an IgG1, IgG2, IgG3 or IgG4 isotype. LNK1 may comprise an amino acid sequence C(X)yC (SEQ ID NO: 48), wherein X is glycine (Gly), serine (Ser), proline (Pro), alanine (Ala), arginine (Arg), asparagine (Asn), aspartic acid (Asp), glutamic acid (Glu), glutamine (Gin), histidine (His), isoleucine (lie), leucine (Leu), lysine (Lys), phenylalanine (Phe), threonine (Thr), tryptophan (Trp) or tyrosine (Tyr), and y is an integer from 1 to 3, further preferably LNK1 comprises an amino acid sequence C(X)yC (SEQ ID NO:48), wherein X is Gly, Ser or Pro, and y is an integer from 1 to 3. Preferably LNK1 comprises the amino acid sequence CPC (SEQ ID NO:49), CGC (SEQ ID NO:50), CSC (SEQ ID NO:51), CPPC (SEQ ID NO:52), CGPC (SEQ ID NO:53), CPGC (SEQ ID NO:54), CGGC (SEQ ID NO:55), CSPG (SEQ ID NO:56), CPSC (SEQ ID NO:57), CSSC (SEQ ID NO:58), CGSC (SEQ ID NO:59), CSGC (SEQ ID NO:60), CPPPC (SEQ ID NO:61), CGPPC (SEQ ID NO:62), CPGPC (SEQ ID NO:63), CPPGC (SEQ ID NO:64), CGGPC (SEQ ID NO:65), CPGGC (SEQ ID NO:66), CGGGC (SEQ ID NO:67), CSPPC (SEQ ID NO:68), CPSPC (SEQ ID NO:69), CPPSC (SEQ ID NO:70), CSSPC (SEQ ID NO:71), CPSSC (SEQ ID NO:72), CSSSC (SEQ ID NO:73), CGSPC (SEQ ID NO:74), CPGSC (SEQ ID NO:75), CSGPC (SEQ ID NO:76) or CPSGC (SEQ ID NO:77).

Preferably the LNK1 may comprise an amino acid sequence selected from any of:

(SEQ ID NO. 17)
GGGSGGSGGCPPCGGSGG,
(SEQ ID NO. 18)
GGGSDDSGGCPPCGGKGG,
and
(SEQ ID NO. 19)
GGAAGGSGGCPPCGGSGG.

Preferably, the first antigen binding domain and/or first and/or second binding region of a variable domain (VD1) and/or (VD2) and/or first antigen binding site (VD1)(VD2), binds with high specificity to the immune effector cell and/or immune effector cell antigen, e.g. with a binding affinity (KD) of about 100 nM or less, about 30 nM or less, about 10 nM or less, about 3 nM or less, about 1 nM or less, for example as measured in by ELISA or SPR or for example as determined by flow cytometry against the effector cell.

In one particular embodiment, the first antigen binding domain and/or first and/or second binding region of a variable domain (VD1) and/or (VD2) and/or first antigen binding site (VD1)(VD2), is capable of binding or specifically binding to an antigen or cell surface antigen expressed on the surface of an immune effector cell, preferably human immune effector cell; optionally wherein said antigen or cell surface antigen is an activating receptor and wherein the binding to said antigen or cell surface antigen or activating receptor results in immune effector cell activation.

Preferably the antigen or cell surface antigen or activating receptor is selected from the group consisting of: CD3, such as the CD3y, CD35, and CD3E chains, CD4, CD7, CD8, CD10, CD11 b, CD11 c, CD14, CD16, CD18, CD22, CD25, CD28, CD32a, CD32b, CD33, CD41, CD41 b, CD42a, CD42b, CD44, CD45RA, CD49, CD55, CD56, CD61, CD64, CD68, CD94, CD90, CD1 17, CD123, CD125, CD134, CD137, CD152, CD163, CD193, CD203c, CD235a, CD278, CD279, CD287, Nkp46, NKG2D, GITR, FCERI, TCRa/β and TCRy/δ, HLA-DR. Preferably the first antigen binding domain and/or first and/or second binding region of a variable domain (VD1) and/or (VD2) and/or first antigen binding site (VD1)(VD2), is specifically reactive to said antigen or cell surface antigen or activating receptor.

In one particular embodiment, the first antigen binding domain and/or first and/or second binding region of a variable domain (VD1) and/or (VD2) and/or first antigen binding site (VD1)(VD2), binds or specifically binds to CD3 or to an antigenic determinant or epitope of the TCR-CD3 complex of human T cells, comprising the peptide chains TCRa, TCR 3, CD3gamma, CD3delta, CD3epsilon, and CD3zeta.

In one particular embodiment, the first binding region of a variable domain (VD1) and the second binding region of a variable domain (VD2) are derived from the humanised anti-CD3 antibody variant, UCHT1, preferably UCHT1.v9 (SEQ ID NO.1 & 2) and specifically binds to CD3 or to an antigenic determinant or epitope thereof. Alternatively, the UCHT1 is UCHT1.v17 (SEQ ID NO. 167 & 168) and specifically binds to CD3 or to an antigenic determinant or epitope thereof.

Preferably the first binding region of a variable domain (VD1) comprises any of one, two or three CDR sequences selected from: SEQ ID NO.3 RASQDIRNYLN, SEQ ID NO.4 YYTSRLES, and SEQ ID NO.5 QQGNTLPWT, or variants thereof. Preferably the second binding region of a variable domain (VD2) comprises any of one, two or three CDR sequences selected from: SEQ ID NO.6 GYTMN, SEQ ID NO.7 LINPYKGVSTYNQKFKD, and SEQ ID NO.8 SGYYGDSDWYFDV, or variants thereof; preferably wherein the variant CDR respectively has 1, 2, or 3 amino acid variations selected from additions, substitutions and deletions with respect to the recited native sequence.

Preferably the first antigen binding domain comprises (a) a first binding region of a variable domain (VD1) which comprises any of one, two or three CDR sequences selected from: SEQ ID NO.3 RASQDIRNYLN, SEQ ID NO.4 YYTSRLES, and SEQ ID NO.5 QQGNTLPWT, or variants thereof and/or (b) a second binding region of a variable domain (VD2) comprises any of one, two or three CDR sequences selected from: SEQ ID NO.6 GYTMN, SEQ ID NO.7LINPYKGVSTYNQKFKD, and SEQ ID NO.8 SGYYGDSDWYFDV, or variants thereof; preferably wherein the variant CDR respectively has 1, 2, or 3 amino acid variations selected from additions, substitutions and deletions with respect to the recited native sequence.

Preferably the first antigen binding domain comprises (a) a first binding region of a variable domain (VD1) which comprises the CDR sequences: SEQ ID NO.3 RASQDIRNYLN, SEQ ID NO.4 YYTSRLES, and SEQ ID NO.5 QQGNTLPWT, or variants thereof and (b) a second binding region of a variable domain (VD2) which comprises the CDR sequences: SEQ ID NO.6 GYTMN, SEQ ID NO.7 LINPYKGVSTYNQKFKD, and SEQ ID NO.8 SGYYGDSDWYFDV, or variants thereof; preferably wherein the variant CDR respectively has 1, 2, or 3 amino acid variations selected from additions, substitutions and deletions with respect to the recited native sequence.

Preferably the first antigen binding domain comprises (a) a first binding region of a variable domain (VD1) which comprises an antibody variable light domain (VL) of SEQ ID NO.1, DIQMTQSPSSLSASVGDRVTITCRASQDIRNYLNWYQQKPGKAPKLLIYYTSRLESGVPSRFSGSGSGTDY TLTISSLQPEDFATYYCQQGNTLPWTFGQGTKVEIK, or variant thereof, or epitope binding portion thereof; and/or (b) a second binding region of a variable domain (VD2) which comprises an antibody variable heavy domain (VH) of SEQ ID NO.2, EVQLVESGGGLVQPGGSLRLSCAASGYSFTGYTMNWVRQAPGKGLEWVALINPYKGVSTYNQKFKDRFT ISVDKSKNTAYLQMNSLRAEDTAVYYCARSGYYGDSDWYFDVWGQGTLVTVSS, or variant thereof, or epitope binding portion thereof.

Preferably the first antigen binding domain comprises (a) a first binding region of a variable domain (VD1) which comprises an antibody variable light domain (VL) of SEQ ID NO.1, DIQMTQSPSSLSASVGDRVTITCRASQDIRNYLNWYQQKPGKAPKLLIYYTSRLESGVPSRFSGSGSGTDY TLTISSLQPEDFATYYCQQGNTLPWTFGQGTKVEIK, or variant thereof, or epitope binding portion thereof; and/or (b) a second binding region of a variable domain (VD2) which comprises an antibody variable heavy domain (VH) of SEQ ID NO.2, EVQLVESGGGLVQPGGSLRLSCAASGYSFTGYTMNWVRQAPGKGLEWVALINPYKGVSTYNQKFKDRFT ISVDKSKNTAYLQMNSLRAEDTAVYYCARSGYYGDSDWYFDVWGQGTLVTVSS, or variant thereof, or epitope binding portion thereof. In respect of the foregoing first antigen binding domain, the chain orientation or connectivity may be (from N-terminal to C-terminal), VD1-VD2, or VD2-VD1, or in the context of the linker LNK1, VD1-LNK1-VD2, or VD2-LNK1-VD1, such that the chain orientations may be VL-VH, or VH-VL, or in the context of the linker LNK1, VL-LNK1-VH, or VH-LNK1-VL. The preferred orientation is VD1-LNK1-VD2, such that from N-terminal to C-terminal the first antigen binding domain orientation is VL-VH, or VL-LNK1-VH.

In respect of the foregoing first antigen binding domain, its binding regions and CDRs the term “variant” encompasses polypeptide sequences having at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% sequence identity to the recited relevant amino acid sequence described. Such variants may include sequences comprising any of amino acid substitutions, deletions, truncations, and insertions of one or more amino acids. Preferably a variant of said first antigen binding domain and/or its first and/or second binding regions and/or the CDRs thereof retain the ability to recognise and bind to the immune effector cell or surface antigen or antigenic determinant thereof, for example CD3. Preferably a variant retains the same or substantially the same functional binding ability of the native sequence for binding and/or activating the immune effector cell or surface antigen or antigenic determinant thereof, for example CD3, for example as measured by binding affinity (KD), i.e. varies by less than or equal to 10%, 7%, 5%, 2% or 1%.

Even further preferred is a bispecific polypeptide molecule according to the invention, wherein said first antigen binding domain and/or first binding site (VD1)(VD2) specifically binds the first antigen, epitope or antigenic determinant of the surface antigen of human immune effector cells (e.g. CD3), and is optionally humanised, has a binding affinity (KD) of about 100 pM or less, about 50 pM or less, about 25 pM or less, or about 10 pM or less. More preferred are high affinity binding sites having binding affinities of about 1 pM or less, about 100 nM or less, about 50 nM or less, about 25 nM or less, about 10 nM or less, about 5 nM or less, about 2 nM or less, about 1 nM or less, about 500 pM or less, about 200 pM or less, about 100 pM or less non-limiting examples of preferred binding affinity ranges for binding sites of the present invention include about 10 pM to about 100 pM, 100 pM to about 1 nM, 1 nM to about 10 nM; about 10 nM to about 20 nM; about 20 nM to about 30 nM; about 30 nM to about 40 nM; about 40 nM to about 50 nM; about 50 nM to about 60 nM; about 60 nM to about 70 nM; about 70 nM to about 80 nM; about 80 nM to about 90 nM; and about 90 nM to about 100 nM, for example as measured by SPR, ELISA or flow cytometry.

Preferably the first antigen-binding domain or first antigen binding site (VD1)(VD2) thereof specifically binds to and/or activates the immune effector cell or surface antigen or receptor thereof which immune effector cell is preferably a T- cell, a CD4+ T-cell, a CD8+ T-cell, a natural killer cell, a macrophage, a granulocyte, or a dendritic cell, preferably a CD8+ T-cell.

### The Second Antigen-Binding Domain

According to the invention the second antigen binding domain may be derived from one or more TCR, which is preferably a human TCR, optionally native or recombinant, which is capable of binding or specifically binding to a second antigen, preferably a cancer or tumour cell antigen, further preferably an MHC-associated antigen or peptide or MHC-associated peptide epitope or antigen. Preferably the second antigen or MHC-associated peptide epitope or antigen is a cancer or tumour cell peptide epitope or antigen and/or wherein the antigen or MHC-associated peptide epitope or antigen is presented or detectibly present on the cell surface of a cancer or tumour cell. Examples of cancer or tumour cell antigens and peptide epitopes or antigens are described herein. Preferably said binding (e.g. of said second antigen binding domain) is specifically reactive to a cancer or tumour cell and is capable of achieving an immune response or achieving immune effector cell activation, preferably to or against said cancer or tumour cell.

Preferably the second antigen-binding domain comprises a first binding region of a variable domain (VR1), preferably of a TCR, and a second binding region of a variable domain (VR2), preferably of a TCR, preferably wherein the first binding region (VR1) and the second binding region (VR2) associate to form a second antigen binding site (VR1)(VR2). In a preferred embodiment said VR1 and VR2 are connected to each other by a second linker (LNK2) connecting said domains wherein the first binding region (VR1) and the second binding region (VR2) associate to form a second antigen binding site (VR1)(VR2).

The order of the binding regions of the second antigen-binding domain can be VR1 N-terminal to VR2 (i.e. N-VR1-VR2) or VR2 N-terminal to VR1 (i.e. N-VR2-VR1), preferably VR1 and VR2 are connected by LNK2, for example from N terminus to C terminus the orientation may be either VR1- LNK2-VR2 or VR2- LNK2-VR1, such that the first binding region (VR1) and the second binding region (VR2) associate to form the second antigen binding site (VR1)(VR2), i.e. connected by LNK2. Preferably the order of the binding regions of the second antigen-binding domain is VR2 N-terminal to VR1 or N-terminal VR2-LNK2-VR1 where VR2 and VR1 are connected by the linker. According to the invention, where the chain order is VR2-VR1, i.e. Vβ-Vα (i.e. Vβ-LNK2-Vα) then the first residue of Vα (i.e. the N-terminal amino acid residue) may optionally be substituted by a glycine residue.

According to the invention, when the order of the binding regions of the second antigen-binding domain is VR2 N-terminal to VR1, i.e. Vβ-Vα (i.e. Vβ-LNK2-Vα), then the VR1 may comprise a Vα domain linked to or fused at its c-terminus to any one of the Ca residue sequences of SEQ ID NO.182, 183 or 184, or for example the respective two N-terminal amino acids contributed by the relevant Ca. According to the invention, when the order of the binding regions of the second antigen-binding domain is VR1 N-terminal to VR2, i.e. Va-Vβ (i.e. Vα-LNK2-Vβ), then the VR1 may comprise a Vα domain linked to or fused at its c-terminus to any one of the Ca residue sequences of SEQ ID NO.185, 186 or 187, or for example the respective four N-terminal amino acids contributed by the relevant Ca. According to the invention, when the order of the binding regions of the second antigen-binding domain is either (a) VR2 N-terminal to VR1, i.e. Vβ-Vα (i.e. Vβ-LNK2-Vα), or (b) VR1 N-terminal to VR2, i.e. Vα-Vβ (i.e. Vα-LNK2-Vβ), then the VR2 may comprise a Vβ domain linked to or fused at its c-terminus to any one of the Cβ residue sequences of SEQ ID NO.189 or 190, or for example the respective four N-terminal amino acids contributed by the relevant Cβ.

Preferably the first binding region of a variable domain (VR1) and/or second binding region of a variable domain (VR2) are derived from one or more TCR which is preferably a human TCR, optionally recombinant or native, which is capable of binding or specifically binding to an MHC-associated peptide or MHC-associated peptide epitope or antigen (pMHC), preferably the MHC-associated peptide epitope or antigen is a cancer or tumour cell peptide epitope or antigen and/or the MHC-associated peptide epitope or antigen is detectibly present on the cell surface of a cancer or tumour cell. Preferably said binding is specifically reactive to a cancer or tumour cell and is capable of achieving an immune response or achieving immune effector cell activation, preferably to or against said cancer or tumour cell, particularly in the context of the bispecific polypeptide molecule of the invention. Preferably said first binding region of a variable domain (VR1) and/or second binding region of a variable domain (VR2) and/or second antigen binding site (VR1)(VR2), have the foregoing binding, binding specificity and said binding is specifically reactive.

According to the invention, the first binding region of a variable domain (VR1) may comprise a TCR α chain variable domain (Vα) or MHC-associated peptide epitope binding portion thereof. Optionally VR1 may further comprise part or all of a TCR α chain constant domain, Cα, preferably linked to or fused to the C-terminus of the Vα domain, for example via a peptide bond, for example VR1 may comprise a Vα domain linked to or fused at its c-terminus to any of 1, 2, 3, 4 or 5 amino acid residues derived from a Cα region of a TCR. Accordingly, and according preferred Cα region amino acids may be selected from any of the sequences NI (SEQ ID NO.182), HI (SEQ ID NO.183), DI (SEQ ID NO.184), NIQN (SEQ ID NO.185), HIQN (SEQ ID NO.186) or DIQN (SEQ ID NO.187) [read N-terminus to C-terminus direction]. Alternatively, the VR1 may lack all or part of said Ca. Preferably VR1 comprises or consists of TCR α chain variable domain (Vα) or MHC-associated peptide epitope binding portion thereof. According to the invention the second binding region of a variable domain (VR2) may comprise a TCR β chain variable domain (Vβ) or MHC-associated peptide epitope binding portion thereof, optionally it may further comprise part or all of TCR β chain constant domain, Cβ, preferably linked to or fused to the C-terminus of the Vβ domain, for example via a peptide bond, for example VR2 may comprise a Vβ domain linked to or fused at its c-terminus to any of 1, 2, 3, 4 or 5 amino acid residues derived from a C3 region of a TCR. Accordingly, and according to the invention preferred C3 region amino acids may be the sequence EDLKN (SEQ ID NO.189) or EDLIN (SEQ ID NO.190), [read N-terminus to C-terminus direction]. Alternatively, the VR2 may lack all or part of said Cβ. Preferably VR2 comprises or consists of a TCR β chain variable domain (Vβ) or MHC-associated peptide epitope binding portion thereof.

The extracellular region of a TCR alpha and beta chain each comprises three CDRs (CDR1, CDR2, CDR3) and four framework regions which are either side of the CDRs (FR1, FR2, FR3, FR4) which constitute the variable region (the variable region having the structure FR1, CDR1, FR2, CDR2, FR3, CDR3, FR4) and a constant region. The constant region or even parts of the constant region may assist in the structural stability of the variable region. In general, the antigen-binding site is formed by the CDR loops of the TCR α-chain and β-chain. CDR3 is the main CDR responsible for recognising HLA-bound antigenic peptides (the usual target for αβ TCRs).

Preferably (a) the first binding region of a variable domain (VR1) may comprise or consist of a TCR α chain variable domain (Vα) or MHC-associated peptide epitope binding portion thereof, which comprises any of one, two or three alpha chain CDR sequences, preferably three a chain CDR sequences, and any of one, two, three or four a chain framework sequences, preferably four a chain framework sequences, and/or (b) the second binding region of a variable domain (VR2) may comprise or consist of a TCR β chain variable domain (Vβ) or MHC-associated peptide epitope binding portion thereof, which comprises any of one, two or three β chain CDR sequences, preferably three β chain CDR sequences, and any of one, two, three or four β chain framework sequences, preferably four β chain framework sequences.

According to the present invention the second antigen-binding domain may comprise a first binding region of a variable domain (VR1) and a second binding region of a variable domain (VR2) and a second linker (LNK2) linking VR1 and VR2 as hereinabove described. Accordingly, the second antigen-binding domain may comprise or consist of any of: (a) a soluble form of a TCR comprising both alpha and beta chains of an extracellular domain wherein each chain comprises three CDRs (CDR1, CDR2, CDR3) and four framework regions (FW1, FW2, FW3, FW4), and a constant region or part of said constant region, (b) a soluble form of a TCR, wherein the TCR comprises a TCR alpha chain variable domain and a TCR beta chain variable domain, (c) a single chain TCR, (scTv) or epitope binding portion thereof, preferably an scTv. Preferably the second antigen-binding domain comprises or consists of an scTv comprising (a) a first binding region of a variable domain (VR1) comprising or consisting of a TCR α chain variable domain (Vα), which comprises three a chain CDR sequences and four a chain framework sequences, and (b) second binding region of a variable domain (VR2) comprising or consisting of a TCR β chain variable domain (Vβ), which comprises three β chain CDR sequences and four β chain framework sequences, wherein said VR1 and VR2 are connected to each other by a second linker (LNK2) connecting said domains optionally wherein the first binding region (VR1) and the second binding region (VR2) associate to form a second antigen binding site (VR1)(VR2); optionally the VR1 and/or VR2 may also comprise any of 1, 2, 3, 4 or 5 amino acid residues derived from a Cα region or Cβ region respectively linked to the c-terminus of the respective Vα or Vβ.

According to the invention the second antigen-binding domain may comprise a first binding region of a variable domain (VR1) of a TCR and a second binding region of a variable domain (VR2) of a TCR and a second linker (LNK2) connecting said domains preferably wherein the first binding region (VR1) and the second binding region (VR2) associate to form a second antigen binding site (VR1)(VR2). The second linker LNK2 may be connected to or fused to VR1 and/or VR2 by a peptide bond, the connection may be to the C-terminal or N-terminal of said VR1 or VR2 depending on the chain orientation of the variable domains. Where VR1 is at the N-terminal then LK2 is connected to the C-terminal of VR1 and the N-terminal of VR2. Where VR2 is at the N-terminal then LK2 is connected to the C-terminal of VR2 and the N-terminal of VR1. In some embodiments LNK2 may be connected to, or peptide bonded to the Vα or Vβ domain or MHC-associated peptide epitope binding portion thereof, in some embodiments LNK2 may be connected to or peptide bonded to amino acid residues derived from a Cα region or Cβ region respectively linked to the c-terminus of the respective Vα or Vβ, for example as already described herein above.

Preferably the linker LNK2 is a flexible linker permitting the connected domains a degree of movement and/or interaction and to achieve appropriate functional separation of the domains, preferably composed of small, non-polar (e.g. glycine) and/or small polar (e.g. serine or threonine) amino acids. Preferably LNK2 is of between 3 and 20 amino acids in length, i.e. any of 3, 4, 5, 6, 7, 8, 9, 10, 11, 12, 13, 14, 15, 16, 17, 18, 19 or 20 amino acids, optionally comprising glycine and serine residues, or glycine and threonine residues. A suitable example of a Gly and Ser containing flexible linker is GSAGSAAGSGEF (SEQ ID NO.37). The linker may also comprise Ala to maintain flexibility, as well as polar amino acids such as Lys and Glu to improve solubility. Preferably the linker is a “GS” linker, i.e. having sequences consisting primarily of stretches of glycine and serine residues, optionally of the motif or sequence (Gly-Gly-Gly-Ser)ₙ (Gly-Gly-Gly-Gly-Ser)ₙ, preferably where the copy number “n”, is any one of 1, 2, 3 or 4, more preferred is either (Gly-Gly-Gly-Ser)₃ or (Gly-Gly-Gly-Ser)₄. In other embodiments the linker may be selected from the sequences KESGSVSSEQLAQFRSLD (SEQ ID NO.38), EGKSSGSGSESKST (SEQ ID NO.39), (Gly)₆ and (Gly)s. Preferably the linker is any of GGGSGGGG (SEQ ID NO.40), GGGS (SEQ ID NO.41), GGGGS (SEQ ID NO.42), TVLRT (SEQ ID NO.43), TVSSAS (SEQ ID NO.44), and TVLSSAS (SEQ ID NO.45), most preferably the linker is GGGGSGGGGSGGGGSGGGGS (SEQ ID NO.46, G4S4 linker) or GSADDAKKDAAKKDGKS (SEQ ID NO.47, GS2 or Kranz linker).

According to one embodiment of the invention VR1 and VR2 may comprise an engineered disulphide bridge capable of introducing a covalent bond between VR1 and VR2, preferably wherein one or more cysteines are recombinantly introduced into a framework region of VR1 and/or VR2, preferably wherein said cysteines are recombinantly introduced into a framework region, preferably a surface exposed framework region, for example into framework region 4 (FR4) in case of Vα and framework region 2 (FR2) in case of Vβ or in the alternative are recombinantly introduced into framework region4 (FR4) in case of Vβ and framework region 2 (FR2) in case of Vα. For example where the chain order in the second antigen binding domain, from N-terminus to C-terminus with respect to the first and second binding regions is VR1-VR2 said cysteines may be recombinantly introduced into for example framework region (FR) 2 in case of the VR1 Vα, and framework region (FR)4 in case of the VR2 VS, or where the chain order from N-terminus to C-terminus is VR2-VR1 said cysteines may be recombinantly introduced for example into framework region (FR) 2 in case of VR2 Vβ and framework region (FR)4 in case of VR1 Vα. Preferably the cysteine substitutions may be made at residue 47 or 48 VR1 Vα and residue 120 in VR2 Vβ or at residue 47 or 48 VR2 Vβ and residue 120 in VR1 Vα where the residue position number is according to the IMGT numbering system for variable domains (imgt.org). For example for an N terminus to C-terminus directions (i) where the chain order is VR1-VR2, i.e. Vα-Vβ (i.e. Vα-LNK2-Vβ) then the cysteine residue substitutions may be Vα residue 47 or 48 and Vβ residue 120; (ii) where the chain order is VR2-VR1, i.e. Vβ-Vα, (i.e. Vβ-LNK2-Vα) then the cysteine residue substitutions may be Vβ residue 47 or 48 and Vα residue 120. According to this embodiment, where the chain order is VR2-VR1, i.e. Vβ-Vα (i.e. Vβ-LNK2-Vα) then the first residue of Vα may optionally be substituted by a glycine residue.

According to this embodiment the linker LNK2 may comprise one or more cysteine (Cys) residues, preferably two cysteine residues, preferably LNK2 comprises a first LNK2 Cys and a second LNK2 Cys, for example LNK2 may comprise the sequence CPPC (SEQ ID NO:52). Accordingly, a first disulphide bond may be formed between a recombinantly introduced VR1 Vα cysteine (Cys) and a first LNK2 Cys and a second disulphide bond may be formed between a recombinantly introduced VR2 Vβ Cys and a second LNK2 Cys; alternatively a first disulphide bond may be formed between a recombinantly introduced VR2 Vβ cysteine (Cys) and a first LNK2 Cys and a second disulphide bond may be formed between a recombinantly introduced VR1 Vα Cys and a second LNK2 Cys. According to this embodiment the cysteine substitutions may be at the IMGT positions 47, 48 and 120 as described herein, i.e., where the chain order is VR1-VR2, i.e. Vα-Vβ (i.e. Vα-LNK2-Vβ) then the cysteine residue substitutions may be Vα residue 47 or 48 and Vβ residue 120; (ii) where the chain order is VR2-VR1, i.e. Vβ-Vα, (i.e. Vβ-LNK2-Vα) then the cysteine residue substitutions may be VS residue 47 or 48 and Vα residue 120. In the alternative the VR2 Vβ Cys and/or VR1 Vα Cys may be native to the sequence, for example a structurally conserved Cys, preferably a structurally conserved surface exposed Vβ Cys and/or Vα Cys. Preferably the linkage formed between the LNK2 first and second Cys and the recombinantly introduced VR1 Vα cysteine (Cys) and recombinantly introduced VR2 Vβ Cys stabilises the second antigen binding site (VR1)(VR2) and/or brings the recombinantly introduced VR1 Vα cysteine (Cys) and recombinantly introduced VR2 Vβ Cys into close proximity, preferably between about 5 to about 12 Angstroms as measured by inter-alpha-carbon distance, preferably about any one of 5, 6, 7, 8, 9, 10, 11, 11.5 or 12 Angstroms.

According to this embodiment the linker LNK2 may comprise one or more cysteine (Cys). Preferably, the LNK2 may comprise a contiguous amino acid sequence derived from an immunoglobulin (Ig) hinge region, optionally the Ig hinge region is derived from a human or a non-human Ig hinge region, preferably human Ig hinge region, further preferably the human Ig hinge region is an IgGI, IgG2, IgG3 or IgG4 isotype. LNK2 may comprise an amino acid sequence C(X)yC (SEQ ID NO: 48), wherein X is glycine (Gly), serine (Ser), proline (Pro), alanine (Ala), arginine (Arg), asparagine (Asn), aspartic acid (Asp), glutamic acid (Glu), glutamine (Gin), histidine (His), isoleucine (lie), leucine (Leu), lysine (Lys), phenylalanine (Phe), threonine (Thr), tryptophan (Trp) or tyrosine (Tyr), and y is an integer from 1 to 3, further preferably LNK2 comprises an amino acid sequence C(X)yC (SEQ ID NO:48), wherein X is Gly, Ser or Pro, and y is an integer from 1 to 3. Preferably LNK2 comprises the amino acid sequence of any of CPC (SEQ ID NO:49), CGC (SEQ ID NO:50), CSC (SEQ ID NO:51), CPPC (SEQ ID NO:52), CGPC (SEQ ID NO:53), CPGC (SEQ ID NO:54), CGGC (SEQ ID NO:55), CSPG (SEQ ID NO:56), CPSC (SEQ ID NO:57), CSSC (SEQ ID NO:58), CGSC (SEQ ID NO:59), CSGC (SEQ ID NO:60), CPPPC (SEQ ID NO:61), CGPPC (SEQ ID NO:62), CPGPC (SEQ ID NO:63), CPPGC (SEQ ID NO:64), CGGPC (SEQ ID NO:65), CPGGC (SEQ ID NO:66), CGGGC (SEQ ID NO:67), CSPPC (SEQ ID NO:68), CPSPC (SEQ ID NO:69), CPPSC (SEQ ID NO:70), CSSPC (SEQ ID NO:71), CPSSC (SEQ ID NO:72), CSSSC (SEQ ID NO:73), CGSPC (SEQ ID NO:74), CPGSC (SEQ ID NO:75), CSGPC (SEQ ID NO:76) or CPSGC (SEQ ID NO:77). Preferably the LNK2 may comprise an amino acid sequence selected from any of: GGGSGGSGGCPPCGGSGG (SEQ ID NO.17), GGGSDDSGGCPPCGGKGG (SEQ ID NO.18), and GGAAGGSGGCPPCGGSGG (SEQ ID NO.19).

In one embodiment LNK2 is either SEQ ID NO.17 or 19, and VR1 comprises a cysteine substitution at position IMGT47 or48 of VR1 Vα, and VR2 comprises a cysteine substitution at position IMGT 120 of VR2 VS, preferably when chain orientation is VR1-LNK2-VR2.

In one embodiment LNK2 is either SEQ ID NO.17 or 18, and VR2 comprises a cysteine substitution at position IMGT46 or47 of VR2 VS, and VR1 comprises a cysteine substitution at position IMGT 120 of VR1 Vα, preferably when chain orientation is VR2-LNK2-VR1.Preferably, the second antigen binding domain and/or first and/or second binding region of a variable domain (VR1) and/or (VR2) and/or second antigen binding site (VR1)(VR2), binds with high specificity to an MHC-associated peptide epitope or antigen (pMHC), e.g., with a binding affinity (KD) of about 100 nM or less, about 30 nM or less, about 10 nM or less, about 3 nM or less, about 1 nM or less, for example as measured in by ELISA or SPR or for example as determined by flow cytometry against a cell, for example a target cancer or tumour cell, presenting said antigen or epitope. Preferably the MHC-associated peptide epitope or antigen is a cancer or tumour cell epitope or antigen and/or the MHC-associated peptide epitope or antigen is presented or detectibly present on the cell surface or a cancer or tumour cell.

In one particular embodiment, the second antigen binding domain and/or first and/or second binding region of a variable domain (VR1) and/or (VR2) and/or second antigen binding site (VR1)(VR2), is capable of binding or specifically binding to an MHC-associated peptide epitope or antigen presented or detectibly present on the cell surface of a cancer or tumour cell wherein the binding to said epitope or antigen is specifically reactive to a cancer or tumour cell and is capable of achieving an immune response or achieving immune effector cell activation, preferably to or against said cancer or tumour cell.

Preferably, the second antigen binding domain and/or first and/or second binding region of a variable domain (VR1) and/or (VR2) and/or second antigen binding site (VR1)(VR2), specifically binds or and/or recognises cancer or tumour target cells or tissues, e.g. cancer cells or tumour cells or tissues for example which present on their surface a MHC-associated peptide epitope or antigen (herein a cancer or tumour peptide antigen or epitope). As used herein, the terms “cancer,” “neoplasm,” and “tumour” are used interchangeably and, in either the singular or plural form, refer to cells that have undergone a malignant transformation that makes them pathological to the host organism.

Examples of such cancer or tumour peptide antigen or epitope are known cancer antigens, for example these may be a cancer-testis antigen, such as NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, HIV-GAG, HIV-TAX, TRAIL, DR4, Thyroglobin, TGFBII frameshift antigen, LAGE-1A, KRAS, CMV (cytomegalovirus), CEA (carcinoembryonic antigen), AFP (a-fetoprotein), MAGE-A1, MAGE-A2, MAGE-A3, MAGE-A4, MAGE-A6, MAGE-A8, and MAGE-A9, MAGE-A10, or MAGE-A12, or peptide epitopes thereof.

In one particular embodiment, the second antigen binding domain and/or first and/or second binding region of a variable domain (VR1) and/or (VR2) and/or second antigen binding site (VD1)(VD2), binds or specifically binds to any one of the above referenced cancer or tumour peptide antigens or epitopes, preferably to PRAME or to a peptide antigen thereof, for example SLLQHLIGL (SEQ ID No.81) or MHC/HLA-A2 presented SLLQHLIGL. According to further alternative embodiments, the second antigen binding domain and/or first and/or second binding region of a variable domain (VR1) and/or (VR2) and/or second antigen binding site (VR1)(VR2), binds or specifically binds to any one of the cancer or tumour peptide antigens or epitopes selected from, SEQ ID NO:128 (HIV-GAG peptide), SEQ ID NO:129 (HIV-TAX peptide) or SEQ ID NO:130 (MAGE-A4 peptide), or respective MHC presented epitope.

The first binding region of a variable domain (VR1) of the second antigen binding domain may comprise a TCR alpha chain variable domain (Vα) or epitope binding portion thereof, optionally it may further comprise part or all of a TCR alpha chain constant domain, Cα, preferably linked to or fused to the C-terminus of the Vα domain. Preferably VR1 comprises or consists of a TCR alpha chain variable domain (Vα) or epitope binding portion thereof. The second binding region of a variable domain (VR2) of the second antigen binding domain may comprise a TCR beta chain variable domain (Vβ) or epitope binding portion thereof, optionally it may further comprise part or all of a TCR beta chain constant domain, Cβ, preferably linked to or fused to the C-terminus of the Vβ domain. Preferably VR2 comprises or consists of an antibody variable heavy domain (Vβ) or epitope binding portion thereof.

Preferably the first binding region of a variable domain (VR1) comprises any of one, two or three CDR sequences selected from: SEQ ID NO.11 DRGSQS, SEQ ID NO.12 IYSNGD, and SEQ ID NO.13 AAVIDNDQGGILT, or variants thereof. Preferably the second binding region of a variable domain (VR2) comprises any of one, two or three CDR sequences selected from: SEQ ID NO.14 PGHRA, SEQ ID NO.15 YVHGEE, and SEQ ID NO.16 ASSPWDSPNVQY, or variants thereof; wherein the variant CDR respectively has 1, 2, or 3 amino acid variations selected from additions, substitutions and deletions with respect to the recited native sequence.

Preferably the second antigen binding domain comprises (a) a first binding region of a variable domain (VR1) which comprises any of one, two or three CDR sequences selected from: SEQ ID NO.11 DRGSQS, SEQ ID NO.12 IYSNGD, and SEQ ID NO.13 AAVIDNDQGGILT, or variants thereof and/or (b) a second binding region of a variable domain (VR2) which comprises any of one, two or three CDR sequences selected from: SEQ ID NO.14 PGHRA, SEQ ID NO.15 YVHGEE, and SEQ ID NO.16 ASSPWDSPNVQY, or variants thereof; wherein the variant CDR respectively has 1, 2, or 3 amino acid variations selected from additions, substitutions and deletions with respect to the recited native sequence.

Preferably the second antigen binding domain comprises (a) a first binding region of a variable domain (VR1) which comprises the CDR sequences SEQ ID NO.11 DRGSQS, SEQ ID NO.12 IYSNGD, and SEQ ID NO.13 AAVIDNDQGGILT, or variants thereof and (b) a second binding region of a variable domain (VR2) which comprises the CDR sequences: SEQ ID NO.14 PGHRA, SEQ ID NO.15 YVHGEE, and SEQ ID NO.16 ASSPWDSPNVQY, or variants thereof; wherein the variant CDR respectively has 1, 2, or 3 amino acid variations selected from additions, substitutions and deletions with respect to the recited native sequence.

Preferably the second antigen binding domain comprises (a) a first binding region of a variable domain (VR1) which comprises or consists of the sequence of SEQ ID NO.9, QKEVEQNSGPLSVPEGAIASLNCTYSDRGSQSFFWYRQYSGKSPELIMSIYSNGDKEDGRFTAQLNKASQ YVSLLIRDSQPSDSATYLCAAVIDNDQGGILTFGTGTRLTIIPNI, or variant thereof, or epitope binding portion thereof; and/or (b) a second binding region of a variable domain (VR2) which comprises or consists of the sequence of SEQ ID NO.10, KAGVTQTPRYLIKTRGQQVTLSCSPIPGHRAVSWYQQTPGQGLQFLFEYVHGEERNKGNFPGRFSGRQF SNSSSEMNISNLELGDSALYLCASSPWDSPNVQYFGPGTRLTVTEDLKN, or variant thereof, or epitope binding portion thereof.

According to a further embodiment the second antigen binding domain may comprise any of:
a. (i) a first binding region of a variable domain (VR1) which comprises any of one, two or three CDR sequences selected from: SEQ ID NO.110, 111, 112 or variants thereof and/or (ii) a second binding region of a variable domain (VR2) which comprises any of one, two or three CDR sequences selected from: SEQ ID NO.113, 114 and 115, or variants thereof;
b. (i) a first binding region of a variable domain (VR1) which comprises any of one, two or three CDR sequences selected from: SEQ ID NO.116, 117, 118 or variants thereof and/or (ii) a second binding region of a variable domain (VR2) which comprises any of one, two or three CDR sequences selected from: SEQ ID NO.119, 120 and 121, or variants thereof; or
c. (i) a first binding region of a variable domain (VR1) which comprises any of one, two or three CDR sequences selected from: SEQ ID NO.122, 123, 124 or variants thereof and/or (ii) a second binding region of a variable domain (VR2) which comprises any of one, two or three CDR sequences selected from: SEQ ID NO.125, 126 and 127, or variants thereof; wherein the variant CDR respectively has 1, 2, or 3 amino acid variations selected from additions, substitutions and deletions with respect to the recited native sequence.

Preferably the second antigen binding domain may comprise any of:
a. (i) a first binding region of a variable domain (VR1) which comprises the CDR sequences SEQ ID NO.110, 111, 112 or variants thereof and (ii) a second binding region of a variable domain (VR2) which comprises any of one, two or three CDR sequences selected from: SEQ ID NO.113, 114 and 115, or variants thereof;
b. (i) a first binding region of a variable domain (VR1) which comprises the CDR sequences SEQ ID NO.116, 117, 118 or variants thereof and (ii) a second binding region of a variable domain (VR2) which comprises the CDR sequences SEQ ID NO.119, 120 and 121, or variants thereof; or
c. (i) a first binding region of a variable domain (VR1) which comprises the CDR sequences SEQ ID NO.122, 123, 124 or variants thereof and (ii) a second binding region of a variable domain (VR2) which comprises the CDR sequences SEQ ID NO.125, 126 and 127, or variants thereof; wherein the variant CDR respectively has 1, 2, or 3 amino acid variations selected from additions, substitutions and deletions with respect to the recited native sequence.

Preferably the second antigen binding domain comprises any of:
(a) (i) a first binding region of a variable domain (VR1) which comprises or consists of the sequence of SEQ ID NO.169, or variant thereof, or epitope binding portion thereof; and/or
(ii) a second binding region of a variable domain (VR2) which comprises or consists of the sequence of SEQ ID NO.170, or variant thereof, or epitope binding portion thereof,
(b) (i) a first binding region of a variable domain (VR1) which comprises or consists of the sequence of SEQ ID NO.171, or variant thereof, or epitope binding portion thereof; and/or
(ii) a second binding region of a variable domain (VR2) which comprises or consists of the sequence of SEQ ID NO.172, or variant thereof, or epitope binding portion thereof, or
(c) (i) a first binding region of a variable domain (VR1) which comprises or consists of the sequence of SEQ ID NO.173, or variant thereof, or epitope binding portion thereof; and/or
(ii) a second binding region of a variable domain (VR2) which comprises or consists of the sequence of SEQ ID NO.174, or variant thereof, or epitope binding portion thereof.

According to the invention the variant of any one of SEQ ID Nos. 9, 169, 171 and 173 may include the addition of the residues QN (SEQ ID NO188), to the C-terminus of said sequence [such that for example the C-terminal ending amino acid sequences of SEQ ID Nos. 9, 169, 171 and 173 are NIQN (SEQ ID NO.185), HIQN (SEQ ID NO.186) or DIQN(SEQ ID NO.187) and NIQN (SEQ ID NO.185)respectively].

In respect of the foregoing second antigen binding domain, the chain orientation or connectivity may be (from N-terminal to C-terminal), VR1-VR2, or VR2-VR1, or in the context of the linker LNK2, VR1-LNK2-VR2, or VR2-LNK2-VR1, the preferred orientation is VR2-LNK2-VR1, such that from-terminal to C-terminal the second antigen binding domain orientation is Vβ-Vα, or Vβ-LNK2-Vα.

In respect of the foregoing second antigen binding domain, its binding regions and CDRs the term “variant” encompasses polypeptide sequences having at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% sequence identity to the recited relevant amino acid sequence described. Such variants may include sequences comprising any of amino acid substitutions, deletions, truncations, and insertions of one or more amino acids. Preferably a variant of said second antigen binding domain and/or its first and/or second binding regions and/or the CDRs thereof retain the ability to recognise and bind to an MHC-associated peptide epitope or antigen presented or detectibly present on the cell surface of a cancer or tumour cell, for example any of PRAME, HIV-TAX, HIV-GAG, or MAGE-A4, e.g. SEQ ID Nos: 81, 128, 129, or 130. Preferably a variant retains the same or substantially the same functional binding ability of the native sequence for the bind an MHC-associated peptide epitope or antigen presented or detectibly present on the cell surface of a cancer or tumour cell, for example any of PRAME, HIV-TAX, HIV-GAG, or MAGE-A4, e.g., SEQ ID NOs: 81, 128, 129, or 130, for example as measured by binding affinity (KD), i.e., varies by less than or equal to 10%, 7%, 5%, 2% or 1% of the recited relevant amino acid sequence described.

Even further preferred is a bispecific polypeptide molecule according to the invention, wherein said second binding site (VR1)(VR2) that specifically binds an MHC-associated peptide epitope or antigen presented or detectibly present on the cell surface of a cancer or tumour cell, has a binding affinity (KD) of about 100 pM or less, about 50 pM or less, about 25 pM or less, or about 10 pM or less. More preferred are high affinity binding sites having binding affinities of about 1 M or less, about 100 nM or less, about 50 nM or less, about 25 nM or less, about 10 nM or less, about 5 nM or less, about 2 nM or less, about 1 nM or less, about 500 pM or less, about 200 pM or less, about 100 pM or less non-limiting examples of preferred binding affinity ranges for binding sites of the present invention include about 10 pM to about 100 pM, 100 pM to about 1 nM, 1 nM to about 10 nM; about 10 nM to about 20 nM; about 20 nM to about 30 nM; about 30 nM to about 40 nM; about 40 nM to about 50 nM; about 50 nM to about 60 nM; about 60 nM to about 70 nM; about 70 nM to about 80 nM; about 80 nM to about 90 nM; and about 90 nM to about 100 nM, for example as measured by SPR, ELISA or flow cytometry.

Preferably, the second antigen binding domain and/or first and/or second binding region of a variable domain (VR1) and/or (VR2) and/or second antigen binding site (VR1)(VR2), binds or specifically binds to the cancer polypeptide antigen or antigenic peptide fragment presented at the cell surface of the tumour cell or cancer cell optionally in complex with a peptide presenting molecule for example major histocompatibility complex (MHC) or an HLA, optionally class I or II, for example with HLA-A2, preferably selected from any of HLA-A*02, HLA-A*02:01, HLA-A*02:02, HLA-A*02:03, HLA-A*02:04, HLA-A*02:05, HLA-A*02:06, or HLA-A*02:07. Optionally, the binding or specific binding is independent of presentation of the cancer polypeptide antigen or antigenic peptide fragment as a complex with a peptide-presenting molecule such as MHC or HLA, for example where the cancer polypeptide antigen or antigenic peptide fragment is expressed directly at the surface of the tumour cell or cancer cell.

Preferably, the second antigen binding domain and/or first and/or second binding region of a variable domain (VR1) and/or (VR2) and/or second antigen binding site (VR1)(VR2), also “specifically recognises” the cancer peptide antigen or epitope presented at the cell surface of a tumour cell or a cancer cell in that it both binds or specifically binds and is specifically reactive to said tumour cell or cancer cell and/or tumour cell or cancer cell presented cancer peptide antigen or epitope, optionally in the context of a peptide presenting molecule.

As used herein with reference to the second antigen binding domain and/or first and/or second binding region of a variable domain (VR1) and/or (VR2) and/or second antigen binding site (VR1)(VR2), the term “epitope binding portion thereof” provides for fragments of the relevant domain which retain the function of cancer-specific binding and/or substantially retain the ability of the domain to bind to and/or be specifically reactive to its target antigen or epitope, for example as presented on the surface of the cancer or tumour cell, for example the binding affinity of the fragment is 75% or more e.g. 80% or more e.g. 85% or more e.g. 90% or more e.g. 95% or more e.g. 98% or more e.g. 99% or more of that of the full domain, for example the respective the TCR Vα and Vβ chains, optionally comprising all or part of a respective Cα and Cβ region or domain.

The binding sites (VD1)(VD2) and (VR1)(VR2) of the present description preferably specifically bind to a surface antigen of immune effector cells and a MHC-associated peptide epitope as herein described, respectively, preferably having a binding affinity (KD) for a MHC-associated peptide epitope and/or an immune effector cells antigen of 100 pM or less.

The binding sites (VD1)(VD2) and (VR1)(VR2) of the present description preferably bind to a MHC-associated peptide epitope and/or an immune effector cells antigen, respectively, with a binding affinity (KD) of about 100 pM or less, about 50 pM or less, about 25 pM or less, or about 10 pM or less. More preferred are high affinity binding sites having binding affinities of about 1 pM or less, about 100 nM or less, about 50 nM or less, about 25 nM or less, about 10 nM or less, about 5 nM or less, about 2 nM or less, about 1 nM or less, about 500 pM or less, about 200 pM or less, about 100 pM or less non-limiting examples of preferred binding affinity ranges for binding sites of the present invention include about 10 pM to about 100 pM, 100 pM to about 1 nM, 1 nM to about 10 nM; about 10 nM to about 20 nM; about 20 nM to about 30 nM; about 30 nM to about 40 nM; about 40 nM to about 50 nM; about 50 nM to about 60 nM; about 60 nM to about 70 nM; about 70 nM to about 80 nM; about 80 nM to about 90 nM; and about 90 nM to about 100 nM, for example as measured by ELISA or SPR or flow cytometry.

### The Hinge Domain

According to the present invention the bispecific polypeptide molecule may comprise a first polypeptide chain comprising a first hinge domain subunit and a second polypeptide chain comprising a second hinge domain subunit, said first and second hinge domain subunits being capable of forming a stable association as a hinge domain. Accordingly, the hinge domain comprises a first hinge domain subunit and a second hinge domain subunit, said first and second hinge domain subunits being capable of forming a stable association as a hinge domain. Preferably the hinge domain comprises two identical hinge domain subunits.

The bispecific polypeptide molecule according to the invention may comprise a hinge domain which comprises a first hinge domain subunit and a second hinge domain subunit, wherein the first hinge domain subunit is fused to the first antigen-binding domain and the second hinge domain subunit is fused to the second antigen-binding domain, wherein the fusion orientates the antigen-binding domain at the N-terminal and the hinge domain subunit at the C-terminal and said fusion is by means of a peptide bond, between the c-terminal amino acid of the respective antigen binding domain and the N-terminal amino acid of hinge domain subunit, optionally wherein said first hinge domain subunit and said second hinge domain subunit is between 2 and 25 amino acids in length and comprises between 1 and 4 cysteine residues.

Preferably the first antigen-binding domain of the bispecific polypeptide molecule is fused to the first hinge domain subunit and the second antigen-binding domain of the bispecific polypeptide molecule is fused to the second hinge domain subunit, preferably wherein the fusion orientates the antigen-binding domain at the N-terminal and the hinge domain subunit at the C-terminal. The fusion may be by means of a peptide bond, for example between the terminal amino acids of the respective antigen binding domain and the hinge domain.

Preferably the hinge domain subunit is between 2 and 25 amino acids in length, i.e. any of 2, 3, 4, 5, 6, 7, 8, 9, 10, 11, 12, 13, 14, 15, 16, 17, 18, 19, 20, 21, 22, 23, 24 or 25 amino acids in length and comprises any of 1, 2, 3 or 4 cysteine residues. In a preferred embodiment the hinge domain subunit is of 11 or 12 amino acids length comprising 2 cysteine residues, in an alternative preferred embodiment the hinge domain subunit is of 4 or 5 amino acids length comprising 2 cysteine residues, in a further preferred embodiment the hinge domain subunit is of 2 amino acids length comprising 2 cysteine residues.

Preferably the hinge domain subunit is from or derived from an antibody hinge domain, preferably an (human) IgG hinge domain selected from IgG1, 2 or 4, or variant sequence thereof. Accordingly, the hinge domain subunit may comprise any one of the sequences selected from any of: SEQ ID No.20 EPKSCDKTHTCPPCPAPELLG (IgG1 Hinge), SEQ ID No.21 ERKCCVECPPCPAPPVAGP (IgG2 Hinge), SEQ ID No.22 ESKYGPPCPSCPAPEFLG (IgG4 Hinge), or variants or fragments thereof (EU numbering used). In some embodiments mutations can be made in hinge domain subunit to remove unwanted antibody-dependent cell-mediated cytotoxicity.

Preferably the hinge domain subunit is a fragment of SEQ ID NO.20, preferably CPPCPAPELLG SEQ ID NO.23 or variant thereof preferably a variant which removes unwanted antibody-dependent cell-mediated cytotoxicity, for example, SEQ ID NO.24 CPPCPAPEAAG or variant or fragment thereof, alternatively the hinge domain subunit may have the sequence of SEQ ID NO.78, 79 or 80 or variant or fragment thereof.

The hinge domain subunit may comprise of consist of SEQ ID NO.80, EPKSSDKTHTCPPCPAPEAAGG, or variant thereof, or an N-terminal truncation thereof such that any of 1, 2, 3, 4, 5, 6, 7, 8, 9 or 10 of the N-terminal amino acid residues are deleted, for example SEQ ID NO.79 represents a truncation of the terminal 5 amino acids of SEQ ID NO.80, and SEQ ID NO.78 represents a truncation of the terminal 10 amino acids of SEQ ID NO.80. Most preferably the hinge domain subunit has the sequence of SEQ ID NO.78.

Preferably the first antigen-binding domain of the bispecific polypeptide molecule is fused to the first hinge domain subunit such that the N-terminal amino acid of the first hinge domain subunit is peptide bonded to the C-terminal amino acid of a first or second binding region of a variable domain (VD1) or (VD2), preferably to the C-terminal amino acid of a variable domain VD1 VL domain or variable domain VD2 VH domain, alternatively to the C-terminal amino acid of a constant domain, CL or part thereof, linked to or fused to the C-terminus of the VD1 VL domain or to the C-terminal amino acid of a constant domain, CH1 or part thereof, linked to or fused to the C-terminus of the VD2 VH domain, most preferably the hinge domain subunit has the sequence of SEQ ID NO.78; and/or the second antigen-binding domain of the bispecific polypeptide molecule is fused to the second hinge domain subunit such that the N-terminal amino acid of the second hinge domain subunit is peptide bonded to the C-terminal amino acid of a first or second binding region of a variable domain (VR1) or (VR2), preferably to the C-terminal amino acid of a variable domain VR1 Vα domain or a variable domain VR2 Vβ domain, alternatively to the C-terminal amino acid of a constant domain, Ca or part thereof, linked to or fused to the C-terminus of the VR1 Vα domain or to the C-terminal amino acid of a constant domain, Cβ or part thereof, linked to or fused to the C-terminus of the VR2 Vβ domain, most preferably the hinge domain subunit has the sequence of SEQ ID NO.78.

The hinge domain subunit may have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence of SEQ ID NO: 20, 21, 22, 23, 24, 78, 79 or 80 or may have any of 1, 2, 3, 4 or 5 amino acid variations selected from additions, substitutions and deletions with respect to the respective recited sequence, preferably wherein the resulting sequence or sequences retain the capability of forming a stable association as a hinge domain for example with an identical hinge domain subunit.

### Connector Sequence (CNX)

According to the present invention the hinge domain provides a connecting sequence (referred to herein by the abbreviation CNX) which connects the first antigen binding domain to the second antigen binding domain, for example, by providing a means of connecting the C-terminal of the first antigen binding domain to the C-terminal amino acid of the second antigen binding domain.

Preferably the hinge domain provides a connecting sequence (CNX) which connects the first antigen binding domain to the second antigen binding domain, such that the first antigen binding domain and the second antigen binding domain may simultaneously bind a first antigen and second antigen respectively, preferably wherein said simultaneous binding provides immune effector cell activation and/or provides immune effector cell target cell killing, preferably wherein said target cell is a cell presenting a cancer or tumour cell antigen, for example a cancer or tumour cell. Preferably the first cell antigen is an activating receptor on an immune effector cell. Preferably the second cell antigen is a target cell antigen, preferably a cancer or tumour antigen. Preferably said simultaneous binding of each antigen is high affinity binding and/or specifically reactive binding.

In one embodiment said connecting sequence (CNX) may consist of a first cysteine residue connected to a second cysteine residue by a disulphide bond (i.e. Cys=Cys, SEQ ID NO.175) wherein the first cysteine residue is connected to the first antigen binding domain and the second cysteine residue is connected to the second antigen binding domain. Preferably said connection between the cysteine residue and the binding domain is by means of a peptide bond. The first cysteine residue may be provided by the first hinge domain subunit of the hinge domain and the second cysteine may be provided by the second hinge domain subunit of the hinge domain.

Preferably the first cysteine is peptide bonded to the C-terminal amino acid of the first antigen binding domain and the second cysteine is peptide bonded to the C-terminal amino acid of the second antigen binding domain.

Accordingly, the first cysteine residue (of CNX) may be peptide bonded to the C-terminal amino acid of a first or second binding region of a variable domain (VD1) or (VD2) of the first antigen binding domain, preferably to the C-terminal amino acid of a variable domain VD1 VL domain or variable domain VD2 VH domain, alternatively to the C-terminal amino acid of a constant domain, CL, or part thereof, linked to or fused to the C-terminus of the VD1 VL domain, or to the C-terminal amino acid of a constant domain, CH, or part thereof, linked to or fused to the C-terminus of the VD2 VH domain; and/or the second cysteine residue (of CNX) may be peptide bonded to the C-terminal amino acid of a first or second binding region of a variable domain (VR1) or (VR2) of the second antigen binding domain, preferably to the C-terminal amino acid of a variable domain VR1 Vα domain or a variable domain VR2 Vβ domain, alternatively to the C-terminal amino acid of a constant domain, Cα, or part thereof, linked to or fused to the C-terminus of the VR1 Vα domain, or to the C-terminal amino acid of a constant domain, Cβ or part thereof, linked to or fused to the C-terminus of the VR2 Vβ domain.

In a preferred embodiment the connecting sequence, CNX, can comprise a sequence (for example Cys=Cys, SEQ ID NO.175) wherein (i) the first cysteine is peptide bonded to a first intervening polypeptide sequence of any one of 1, 2, 3, 4, 5, 6, 7, 8, 9, or 10 amino acid residues which intervene the first cysteine residue and the first antigen binding domain or C-terminal amino acid of the first antigen binding domain; and/or (ii) the second cysteine is peptide bonded to a second intervening polypeptide sequence of any one of 1, 2, 3, 4, 5, 6, 7, 8, 9, or 10 amino acid residues which intervene the second cysteine and the second antigen binding domain or C-terminal amino acid of the second antigen binding domain. Preferably the length of the first and second intervening polypeptide sequences are the same. For example, a combined intervening sequence and cysteine residue may have the sequence of the N-terminal portion of any one of the hinge domain or hinge domain subunits disclosed herein.

According to this embodiment, the terminal amino acid (e.g., N-terminal amino acid) of the first intervening polypeptide sequence is peptide bonded to the C-terminal amino acid of the first antigen binding domain and/or the terminal amino acid (e.g., N-terminal amino acid) of the second intervening polypeptide sequence is peptide bonded to the C-terminal amino acid of the second antigen binding domain. According to this embodiment, the terminal amino acid (e.g., N-terminal amino acid) of the first intervening polypeptide sequence (of CNX) may be peptide bonded to the C-terminal amino acid of a first or second binding region of a variable domain (VD1) or (VD2) of the first antigen binding domain, preferably to the C-terminal amino acid of a variable domain VD1 VL domain or variable domain VD2 VH domain, alternatively to the C-terminal amino acid of a constant domain, CL or part thereof, linked to or fused to the C-terminus of the VD1 VL domain or to the C-terminal amino acid of a constant domain, CH1 or part thereof, linked to or fused to the C-terminus of the VD2 VH domain; and/or the terminal amino acid (e.g., N-terminal amino acid) of the second intervening polypeptide sequence (of CNX) may be peptide bonded to the C-terminal amino acid of a first or second binding region of a variable domain (VR1) or (VR2) of the second antigen binding domain, preferably to the C-terminal amino acid of a variable domain VR1 Vα domain or a variable domain VR2 Vβ domain, alternatively to the C-terminal amino acid of a constant domain, Ca or part thereof, linked to or fused to the C-terminus of the VR1 Vα domain or to the C-terminal amino acid of a constant domain, Cβ or part thereof, linked to or fused to the C-terminus of the VR2 Vβ domain.

According to the foregoing embodiments the first antigen binding domain may be connected to the second antigen binding domain by a connection sequence or connector sequence (CNX), such that the C-terminal end of the first antigen binding domain is separated from the C-terminal end of the second antigen binding domain by any of 2, 3, 4, 5, 6, 7, 8, 9, 10, 11, 12, 13, 14, 15, 16, 17, 18, 19, 20, 21, 22, 23 or 24 peptide bonds in combination with 1 disulphide bond (e.g. cys=cys), or by a distance of any of about 1.36, 1.74, 2.12, 2.5, 2.88, 3.26, 3.64, 4.02, 4.4, 4.78, 5.16, 5.54, 5.92, 6.3, 6.68, 7.06, 7.44, 7.82, 8.2, 8.58, 8.96, 9.34 or about 9.72 nm (or said distance in nm to one decimal place), said distances in nm being the sum of alpha-carbon (CA) to alpha-carbon distances (0.38 nm) plus the distance of between the two alpha carbons of the first and second cysteine residues (cys=cys) (0.6 nm). Preferably the C-terminal end of the first antigen binding domain is separated from the C-terminal end of the second antigen binding domain by any of 2, 3, 4, 5, 6, 7, 8, peptide bonds in combination with 1 disulphide bond (e.g. cys=cys), or by a distance of any of about 1.36, 1.74, 2.12, 2.5, 2.88, 3.26, or about 3.64, nm (or said distance in nm to one decimal place).

Preferably the C-terminal end of the first antigen binding domain is separated from the C-terminal end of the second antigen binding domain by any one of 2, 4, 6 or 8 peptide bonds in combination with 1 disulphide bond (e.g. cys=cys) or by any of around 1.36, 2.12, 2.88 or around 3.64 nm (or said distance in nm to one decimal place), optionally wherein the CNX sequence is any of SEQ ID NOs. 175, 176, 177 or 178. Preferably the C-terminal end of the first antigen binding domain is separated from the C-terminal end of the second antigen binding domain by 2 peptide bonds in combination with 1 disulphide bond (e.g. cys=cys) or around 1.36 nm (2×0.38 nm+0.6 nm), preferably wherein said CNX is provided by two cysteine residues connected by a disulphide bond (cys=cys) SEQ ID NO.175.

According to the foregoing embodiments the first antigen binding domain may be connected to the second antigen binding domain by a connection sequence or connector sequence (CNX), such that (e.g., depending on the relative variable domain orientation in the first binding domain, and the relative variable domain orientation in the second binding domain) any one of:
(a) the C-terminal end amino acid of the first antigen binding domain second variable domain VD2 VH domain is separated from the C-terminal end amino acid of the second antigen binding domain first variable domain VR1 Vα domain,
(b) the C-terminal end amino acid of the first antigen binding domain second variable domain VD2 VH domain is separated from the C-terminal end amino acid of the second antigen binding domain second variable domain VR2 Vβ domain,
(c) the C-terminal end amino acid of the first antigen binding domain first variable domain VD1 VL domain is separated from the C-terminal end amino acid of the second antigen binding domain first variable domain VR1 Vα domain, or
(d) the C-terminal end amino acid of the first antigen binding domain first variable domain VD1 VL domain is separated from the C-terminal end amino acid of the second antigen binding domain second variable domain VR2 Vβ domain, by any of, 1, 2, 3, 4, 5, 6, 7, 8, 9, 10, 11, 12, 13, 14, 15, 16, 17, 18, 19, 20, 21, 22, 23 or 24 peptide bonds in combination with 1 disulphide bond, or by a distance of any of about, 0.98 n m, 1.36, 1.74, 2.12, 2.5, 2.88, 3.26, 3.64, 4.02, 4.4, 4.78, 5.16, 5.54, 5.92, 6.3, 6.68, 7.06, 7.44, 7.82, 8.2, 8.58, 8.96, 9.34 or 9.72 nm (or said distance in nm to one decimal place).

Preferably the C-terminal end amino acid of the first antigen binding domain second variable domain VD2 VH domain is separated from the C-terminal end amino acid of the second antigen binding domain first variable domain VR1 Vα domain by any one of by any of, 1, 2, 3, 4, 5, 6, 7, 8, 9, 10, 11, 12, 13 peptide bonds in combination with 1 disulphide bond, or by any of around, 0.98 nm, 1.36, 1.74, 2.12, 2.5, 2.88, 3.26, 3.64, 4.02, 4.4, 4.78, 5.16, or 5.54 nm (or said distance in nm to one decimal place), optionally wherein the CNX sequence is any of SEQ ID NOs. 175, 176, 177 or 178; more preferably by 4 peptide bonds and 1 disulphide bond or by around 2.12 nm, preferably wherein said CNX is provided by two cysteine residues connected by a disulphide bond (cys=cys) SEQ ID NO.175.

According to the present invention the hinge domain, of the bispecific molecule of the invention, which provides a connecting sequence (referred to herein by the abbreviation CNX) which connects the first antigen binding domain to the second antigen binding domain, may comprise a hinge domain subunit sequence selected from any of sequences, SEQ ID NO.24, 78, 79 or 80, preferably SEQ ID NO.78.

According to invention the CNX may comprise or consist of the sequence of any one of SEQ ID NOs. 175, 176, 177, 178, 179, 180 or 181, preferably SEQ ID NO. 175. The connector sequence, CNX, determines the degree of separation of the first and second antigen binding domains in the bispecific polypeptide molecule of the invention, this in turn determines the synapse also known as the immunological synapse, a critical structural feature that forms the interface between antigen presenting cells (APC)/tumour cell (bound by the second antigen binding domain) and the recruited immune effector cell, for example T lymphocyte (bound by the first antigen binding domain) and is required for the establishment of an effective immune effector cell activation, for example T cell activation, and immune response to the targeted cancer or tumour cell, it is also required for the development of long-lasting immune effector cell or T cell memory. The connector is therefore important to immune effector cell activation.

According to the invention the hinge domain provides a connecting sequence (CNX) wherein the CNX connects the first antigen binding domain to the second antigen binding domain, such that the distance between the first antigen binding domain and the second antigen binding domain permits the first antigen binding domain and the second antigen binding domain to simultaneously bind a first antigen and second antigen respectively, preferably wherein said simultaneous binding provides immune effector cell activation and/or provides immune effector cell target cell killing, preferably wherein said target cell is a cell presenting a cancer or tumour cell antigen, for example a cancer or tumour cell.

### The Fc Domain

According to the present invention the bispecific polypeptide molecule may comprise a first polypeptide chain comprising a first Fc domain subunit and a second polypeptide chain comprising a second Fc domain subunit, said first and second Fc domain subunits being capable of forming a stable association as an Fc domain or Fc domain portion.

Preferably the first antigen-binding domain of the bispecific polypeptide molecule is fused to the first hinge domain subunit and the second antigen-binding domain of the bispecific polypeptide molecule is fused to the second hinge domain subunit, preferably wherein the fusion orientates the antigen-binding domain at the N-terminal and the hinge domain subunit at the C-terminal.

An Fc domain subunit may be fused to a hinge domain subunit or may comprise the hinge domain subunit, for example with the hinge domain subunit sequence fused or comprised N-terminal to the Fc domain subunit sequence. For example, a Fc domain subunit may be fused to a hinge domain subunit by means of a peptide bond, for example between the terminal amino acid residue of said hinge domain subunit and Fc domain subunit.

Preferably (a) the first antigen-binding domain of the bispecific polypeptide molecule is fused to the first hinge domain subunit and said first hinge domain subunit is fused to and/or comprised in a first Fc domain subunit and (b) the second antigen-binding domain of the bispecific polypeptide molecule is fused to the second hinge domain subunit and said second hinge domain subunit is fused to and/or comprised in a second Fc domain subunit; preferably wherein the fusion orientates the antigen-binding domain at the N-terminal of the hinge domain subunit and the hinge domain subunit at the N-terminal of the Fc domain subunit, preferably wherein the first and second Fc domain subunits may associate to be capable of forming a stable association as an Fc domain or Fc domain portion

According to an embodiment the present invention the Fc domain subunit or Fc domain may be an Fc domain subunit portion or Fc domain portion such that the full length sequence may be truncated at the N-terminus or C-terminus of the sequence, preferably at the C-terminus, for example at the C-terminal end, for example wherein any one of 1, 2, 3, 4, 5, 6, 7, 8, 9 or 10 amino acids are deleted from the N or C-terminus, preferably from the C-terminal end, preferably 2 amino acids are deleted, preferably from the C-terminal end. For example, the portion of an Fc domain subunit may be truncated at the C-terminus by deletion of one or more amino acids preferably 2 amino acids, for example, preferably the terminal GK amino acids are deleted. Preferably the portion is a dimerising portion, for example which retains the capability of the Fc domain subunit or Fc domain subunit portion to form a stable association as an Fc domain or Fc domain portion such that the two polypeptide chains of the bispecific polypeptide molecule are connected by covalent and/or non-covalent bonds between the Fc-domain subunits and/or hinge domain subunits.

Preferably the Fc domain or portion thereof is an antibody or immunoglobulin Fc domain or portion, preferably human or humanised, native or recombinant in origin, further preferably derived from an IgG1, IgG2 or IgG4 Fc domain or portion, or a chimera of any two or three of an IgG1, IgG2 or IgG4 Fc domain or portion, most preferably IgG1 or human IgG1, preferably wherein the portion is a dimerising portion as described herein.

The Fc domain subunit or portion thereof may comprise one, two or three heavy chain constant domains (denoted CH domains, i.e., CH2, CH3, and CH4) in each polypeptide chain. Preferably the Fc domain subunit or portion comprises an N-glycosylation site which permits Fc receptor-mediated activity. Preferably the Fc domain subunit or portion thereof improves the stability and/or pharmacokinetic properties of the bispecific polypeptide molecule and/or extends the serum half-life thereof, for example as compared to a bispecific polypeptide molecule not comprising the Fc subunit or portion, it is desirable for the selected sequence of the Fc subunit or portion to optimise this function to mitigate against fast clearance and a short half-life of the molecule in-vivo.

The Fc domain subunit or subunit domain or portion thereof may comprise one or more mutations from the native sequence, i.e. it may be a variant sequence. Preferably the Fc domain comprises mutations which increase the binding affinity to neonatal Fc receptor (FcRn) and/or half-life, e.g., in-vivo/in serum, preferably these mutations are located in the Fc CH2 and CH3 domains, preferably at the interface between the CH2 and CH3 domains, preferably the mutations are selected from any one or more of: T250Q, M428L, M252Y, S254T, T256E, H433K and N434F, further preferably selected from any one or more of the groups: T250Q/M428L, M252Y/S254T/T256E, and H433K/N434F, preferably comprising T250Q/M428L or M252Y/S254T/T256E/H433K/N434F, alternatively comprising T250Q/M428L/M252Y/S254T/T256E/H433K/N434F (in accordance with the EU numbering system).

Preferably the Fc domain or domain subunit or portion thereof can comprise a CH2 domain comprising at least one effector function silencing mutation. Preferably, these mutations are introduced into the residue region 233-238 (EU numbering), 246-251 (Kabat numbering) ELLGGP (SEQ ID No.82) of human IgG1, or corresponding residues of other IgG isotypes. Preferably the mutations are selected from one or more of: E233P, L234V, L235A and no residue or G in position 236 (EU numbered), [E246P, L247V, L248A and no residue or G in position 249 (Kabat)]. These preferred mutations represent the substitution of one or more mutations corresponding to residues derived from IgG2 and/or IgG4 into IgG1 Fc. The Fc domain or domain subunit or portion thereof can comprise or further comprise the mutation P331 S (EU numbering).

In a preferred embodiment the bispecific polypeptide molecule comprises a first and/or second Fc domain subunit or portion thereof, wherein the Fc domain subunit or portion comprises at least one effector function silencing mutation at a residue selected from positions 233, 234, 235, 236, 297 and 331, preferably wherein said effector function silencing mutation is generated by replacing at least one residue in position 233, 234, 235, 236, and 331 with the corresponding residue derived from IgG2 or IgG4.

The Fc domain or domain subunit or portion thereof can comprise a chimeric domain which is derived from two or more human immunoglobulin heavy chain CH2 domains, which human immunoglobulins are selected from IgG1, IgG2 and IgG4,and wherein the chimeric domain is a human immunoglobulin heavy chain CH2 domain which has any one or more of amino acid mutations at the stated positions: 233P, 234V, 235A and no residue or G in position 236, 327G, 330S and 331S (EU numbering system), preferably all of the substitutions 233P, 234V, 235A, 236G, 327G, 330S and 331S, and/or is at least 98% identical to a CH2 sequence (residues 231-340) from human IgG1, IgG2 or IgG4 having said modified amino acids. Optionally position 297 carries an N-glycosylated variant or a residue selected from the group of A, G and Q, i.e., a deglycosylated variant.

Preferred CH2 partial sequences of the Fc domain or domain subunit or part thereof may be selected from any of the following sequences:

(SEQ ID NO. 32)
APELLGGPSVFLFPPKPKDTLMISRTPEVTCVVVDVSHEDP
EVKFNWYVDGVEVHNAKTKPREEQYNSTYRVVSVLTVLHQD
WLNGKEYKCKVSNKALGAPIEKTISKAK,
[CH2 region residues 231-340 IgG1, EU numbering]
(SEQ ID NO. 33)
APPVA-GPSVFLFPPKPKDTLMISRTPEVTCWVDVSHEDPE
VKFNWYVDGVEVHNAKTKPREEQYQSTYRVVSVLTVLHQDW
LNGKEYKCKVSNKALPASIEK-;
(SEQ ID NO. 34)
APPVA-GPSVFLFPPKPKDTLMISRTPEVTCWVDVSHEDP
EVKFNWYVDGVEVHNAKTKPREEQYNSTYRWSVLTVLHQD
WLNGKEYKCKVSNKALPASIEK-.

In a preferred embodiment of the bispecific polypeptide molecule of the invention, the Fc domain or domain subunit can comprise a CH3 domain comprising at least one mutation facilitating dimerization of the Fc domain, preferably by introduction of “knobs-into-holes” mutations which can be engineered into the Fc sequence. With this design, Fc domains are driven to form heterodimers instead of their normal homodimers by addition of protruding bulky hydrophobic residues (“knobs”) to one chain and creation of complementary hydrophobic pockets (“holes”) on the other. A ‘knob’ variant can be obtained by replacement of a small amino acid with a larger one to insert into a ‘hole’ in the opposite domain created by replacement of a large residue with a smaller one (Ridgway, J. B. B. et al. “Knobs-into-holes” engineering of antibody CH3 domains for heavy chain heterodimerization. Protein Eng. 1996, 9, 617-621). Preferably the knob mutation is in the first polypeptide chain Fc domain and the hole mutation is in the second polypeptide chain Fc domain, alternatively the knob mutation is in the second polypeptide chain Fc domain and the hole mutation is in the first polypeptide chain Fc domain.

Preferably a bispecific polypeptide molecule according to the invention is provided, wherein in the CH3 domain of a first Fc domain subunit or portion thereof comprises a sequence in which an amino acid residue is replaced with an amino acid residue having a larger side chain volume, thereby generating a protuberance within the CH3 domain of the said subunit which is positionable in a cavity within the CH3 domain of a second Fc domain subunit or portion thereof, and which in the CH3 domain of the said Fc domain subunit or portion thereof an amino acid residue is replaced with an amino acid residue having a smaller side chain volume, thereby generating a cavity within the CH3 domain of the subunit within which the protuberance within the CH3 domain of the first subunit is positionable. Preferably said “protuberance and cavity” or “knob and hole” mutations facilitate the formation of heterodimers between the first polypeptide chain and second polypeptide chain of the bispecific polypeptide molecule of the present invention. Alternatively, the protuberance and cavity alterations are exchanged between the above order with respect to the first and second Fc domain subunit or portion thereof.

Preferably the knob-into-hole mutations are selected from (EU numbering): T366Y or T366W and/or S354C as knob, and any one two or three of: T366'S, L368′A, Y407V or Y407Y, and/or Y349C as hole in the CH3 domain. Further preferably the knob-into-hole mutations may further include either or both of the mutations K409A and F405′K (Wei et al. Structural basis of a novel heterodimeric Fc for bispecific antibody production, Oncotarget. 2017). A preferred embodiment is knob T366Y and hole Y407Y. A further preferred embodiment is S354C and T366W knob mutations and Y349C and T366S as hole mutations.

In a further preferred embodiment, the bispecific polypeptide molecule of the invention, can comprise or further comprise recombinantly introduced cysteine bridges between at least one cysteine residue on the first polypeptide chain and at least one cysteine residue on the second polypeptide chain in order to improve the stability of the molecules and heterodimerisation, optimally without interfering with the binding characteristics of the bivalent molecule, and/or for improved heterodimerization. Preferably a single cysteine residue is introduced into the Fc domain of both the knob and hole chains, preferably in the CH3 domain, preferably facilitating a disulphide bridge between said domain subunits. Preferably the Fc domain comprises a CH3 domain comprising at least one mutation introducing a non-native cysteine residue, for example selected from the mutations S354C and/or Y349C.

In a preferred embodiment a bispecific polypeptide molecule according to the invention is provided, wherein the Fc domain or domain subunit comprises a CH3 domain comprising at least one mutation that facilitates the formation of heterodimers, for example, wherein the mutations are located at any position selected from 366, 368, 405, and 407, preferably, wherein said mutations comprise T366W and T366'S, L368A′ and Y407V as knob-into-hole mutations (EU numbering) and/or wherein the Fc domain comprises CH2 and CH3 domain(s) comprising at least two additional cysteine residues, for example S354C and Y349C or L242C and K334C (EU numbering).

In a preferred embodiment a bispecific polypeptide molecule of the invention is provided wherein the first polypeptide chain comprises an Fc domain subunit or portion thereof comprising the mutations L234A L235A S354C T366W (Fc knob scFv arm) and the second polypeptide chain comprises an Fc domain subunit or portion thereof comprising the mutations L234A L235A Y349C T366S L368A and Y407V (Fc hole scTv arm). Alternatively, preferably an embodiment of a bispecific polypeptide molecule of the invention is provided wherein the second polypeptide chain comprises an Fc domain subunit or portion thereof comprising the mutations L234A L235A S354C T366W (Fc knob scTv arm) and the first polypeptide chain comprises an Fc domain subunit or portion thereof comprising the mutations L234A L235A Y349C T366S L368A and Y407V (Fc hole scFv arm). In each case, the Fc sequence is preferably an IgG1 Fc sequence, i.e. said Fc domain subunit or portion thereof is derived from IgG1. According to these embodiments the portion of said Fc domain subunit may be truncated at the C-terminus by deletion of one or more amino acids preferably 2 amino acids, preferably the terminal GK amino acids are deleted.

According to the present invention the Fc domain subunit or portion thereof may have a sequence selected from any one of:

SEQ ID NO. 25
CPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALGAPIEKTISKAKGQPREPQVYTLPPCRDELTKNQVSL
WCLVKGFYPSDIAVEWESNGQPENNYKTTPPVLDSDGSFFLYSKLTVDKSRWQQGNVFSCSVMHEALH
NHYTQKSLSLSP,
(Fc Knob, including hinge, truncated with C-terminal GK
deletion);
SEQ ID NO. 26
PSVFLFPPKPKDTLMISRTPEVTCVVVDVSHEDPEVKFNWYVDGVEVHNAKTKPREEQYNSTYRVVSVLT
VLHQDWLNGKEYKCKVSNKALGAPIEKTISKAKGQPREPQVYTLPPCRDELTKNQVSLWCLVKGFYPSDIA
VEWESNGQPENNYKTTPPVLDSDGSFFLYSKLTVDKSRWQQGNVFSCSVMHEALHNHYTQKSLSLSP,
(Fc Knob, excluding hinge, truncated with C-terminal GK deletion);
SEQ ID NO. 27
CPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALGAPIEKTISKAKGQPREPQVCTLPPSRDELTKNQVSL
SCAVKGFYPSDIAVEWESNGQPENNYKTTPPVLDSDGSFFLVSKLTVDKSRWQQGNVFSCSVMHEALHN
HYTQKSLSLSP, (Fc Hole, including hinge, truncated with C-terminal GK
deletion);
SEQ ID NO. 28
PSVFLFPPKPKDTLMISRTPEVTCVVVDVSHEDPEVKFNWYVDGVEVHNAKTKPREEQYNSTYRVVSVLT
VLHQDWLNGKEYKCKVSNKALGAPIEKTISKAKGQPREPQVCTLPPSRDELTKNQVSLSCAVKGFYPSDIA
VEWESNGQPENNYKTTPPVLDSDGSFFLVSKLTVDKSRWQQGNVFSCSVMHEALHNHYTQKSLSLSP,
(Fc Knob, excluding hinge, truncated with C-terminal GK deletion);
or sequence variant including the amino acid residues GK at
the C-terminus.

In a preferred embodiment, a bispecific polypeptide molecule of the invention is provided wherein the first polypeptide chain comprises an Fc domain subunit or portion thereof comprising either sequence of SEQ ID No.25 or 26; and/or the second polypeptide chain comprises an Fc domain subunit or portion thereof comprising either sequence of SEQ ID No.27 or 28, optionally wherein said recited sequences may further comprise the amino acid residues GK at the C-terminus.

Preferably the first polypeptide chain comprises an Fc domain subunit or portion thereof comprising the sequence of SEQ ID No.25 and the second polypeptide chain comprises an Fc domain subunit or portion thereof comprising a sequence of SEQ ID No.27, alternatively preferably the first polypeptide chain comprises an Fc domain subunit or portion thereof comprising the sequence of SEQ ID No.26 and the second polypeptide chain comprises an Fc domain subunit or portion thereof comprising a sequence of SEQ ID No.28; optionally wherein said recited sequences may further comprise the amino acid residues GK at the C-terminus.

Alternatively preferably the second polypeptide chain comprises an Fc domain subunit or portion thereof comprising the sequence of SEQ ID No.25 and the first polypeptide chain comprises an Fc domain subunit or portion thereof comprising either sequence of SEQ ID No.27, alternatively preferably the second polypeptide chain comprises an Fc domain subunit or portion thereof comprising the sequence of SEQ ID No.26 and the first polypeptide chain comprises an Fc domain subunit or portion thereof comprising a sequence of SEQ ID No.28; optionally wherein said recited sequences may further comprise the amino acid residues GK at the C-terminus.

According to the invention the Fc domain subunit or portion thereof may be a variant having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to the amino acid sequence to the respective sequence of SEQ ID NO: 25, 26, 27 or 28, preferably wherein said Fc domain subunit or portion thereof may comprise amino acid variations selected from additions, substitutions and deletions with respect to the recited native sequence, for example 1, 2, 3, 4, 5, 6, 7, 8, 9, or 10 such variations. Preferably said variant Fc domain subunit or portion thereof retains the capability of forming a stable association between the first and second Fc domain subunits and are capable of forming a stable association as an Fc domain or Fc domain portion such that the two polypeptide chains are connected by covalent and/or non-covalent bonds between the Fc-domain subunits.

### The Bispecific Polypeptide Molecule

In a preferred embodiment the bispecific polypeptide molecule of the invention comprises a first polypeptide chain and a second polypeptide chain, wherein
(A) the first polypeptide chain comprises in N-terminal to C-terminal direction: (i) a first antigen-binding domain, (ii) a first hinge domain subunit, (iii) a first Fc domain subunit, wherein the first antigen-binding domain comprises in N-terminal to C-terminal direction an antibody variable light domain (VL) or epitope binding portion thereof, a first linker LNK1, and an antibody variable heavy domain (VH) or antigen binding portion thereof; and
(B) the second polypeptide chain comprises in N-terminal to C-terminal direction: (i) a second antigen-binding domain, ii) a second hinge domain subunit, (iii) a second Fc domain subunit, wherein the second antigen-binding domain comprises in N-terminal to C-terminal direction; TCR β chain variable domain (Vβ) or MHC-associated peptide epitope binding portion thereof, a second linker LNK2, a TCR α chain variable domain (Vα) or MHC-associated peptide epitope binding portion thereof;
preferably wherein the first and second hinge domain subunits are capable of forming a stable association as a hinge domain and the first and second Fc domain subunits are capable of forming a stable association as an Fc domain or Fc domain portion such that the two polypeptide chains are connected by covalent and/or non-covalent bonds between the hinge domain subunits and Fc-domain subunits. According to an alternative of this preferred embodiment the second antigen-binding domain may comprise in N terminal to C-terminal direction; TCR α chain variable domain (Vα) or MHC-associated peptide epitope binding portion thereof, a second linker LNK2, and a TCR β chain variable domain (Vβ) or MHC-associated peptide epitope binding portion thereof.

According to this embodiment the first binding region of a variable domain (VR1) may further comprise part or all of a TCR α chain constant domain, Cα, preferably linked to or fused to the C-terminus of the Vα domain, for example via a peptide bond, as herein described and/or the second binding region of a variable domain (VR2) may further comprise part or all of TCR β chain constant domain, Cβ, preferably linked to or fused to the C-terminus of the Vβ domain, for example via a peptide bond, as herein described. In a preferred embodiment the bispecific polypeptide molecule of the invention comprises a first polypeptide chain and a second polypeptide chain, wherein:
(a) the first polypeptide chain comprises the sequence SEQ ID No.29, and the second polypeptide chain comprises the sequence SEQ ID No.30, optionally wherein said sequences may further comprise the amino acids GK at the C-terminus, or
(b) the first polypeptide chain comprises the sequence SEQ ID No.29, and the second polypeptide chain comprises the sequence SEQ ID No.31, optionally wherein said sequences may further comprise the amino acids GK at the C-terminus.

In further embodiments the bispecific polypeptide molecule of the invention comprises a first polypeptide chain and a second polypeptide chain, wherein:
(a) the first polypeptide chain comprises the sequence SEQ ID No.105, and the second polypeptide chain comprises the sequence SEQ ID No.83 or SEQ ID 84,
(b) the first polypeptide chain comprises the sequence SEQ ID No.106, and the second polypeptide chain comprises the sequence SEQ ID No.91,
(c) the first polypeptide chain comprises the sequence SEQ ID No.29, and the second polypeptide chain comprises any one of the sequences selected from SEQ ID No.85, 86, 87, 88, 89, and 90;
(d) the first polypeptide chain comprises the sequence SEQ ID No.29, and the second polypeptide chain comprises any one of the sequences selected from SEQ ID No.92, 93, 94, and 95;
(e) the first polypeptide chain comprises the sequence SEQ ID No.29, and the second polypeptide chain comprises any one of the sequences selected from SEQ ID No.96, 97, 98, 99, and 100;
(f) the first polypeptide chain comprises the sequence SEQ ID No.29, and the second polypeptide chain comprises any one of the sequences selected from SEQ ID No.101, 102, 103 or 104,
(g) the first polypeptide chain comprises the sequence SEQ ID No.29, and the second polypeptide chain comprises any one of the sequences selected from SEQ ID No.136, 137, 138, 139, 140, or
(h) the first polypeptide chain comprises the sequence SEQ ID No.29, and the second polypeptide chain comprises any one of the sequences selected from SEQ ID No.141, 142, 143, 144 or 105, optionally wherein said sequences may further comprise the amino acids GK at the C-terminus.

The present disclosure provides for a first polypeptide chain and/or second polypeptide chain having at least 50%, at least 60%, at least 70%, at least 80%, at least 90%, at least 95%, at least 96%, at least 97%, at least 98%, or at least 99% sequence identity to an amino acid sequence selected from any of the above stated first and/or second polypeptide chain sequences respectively as described herein. In a preferred embodiment, the percent identity of at least 50%, at least 60%, at least 70%, at least 80%, at least 90%, at least 95%, at least 96%, at least 97%, at least 98%, or at least 99% applies to any of the sequences of the structural regions described in herein and comprised in said first and/or second polypeptide chain sequence, for example, VD1, LNK1, VD2, Hinge Domain Subunit 1, Fc Domain Subunit 1; VR1, LNK2, VR2, Hinge Domain Subunit 2, Fc Domain Subunit 2, as described or being part of the sequences as disclosed herein.

### Sequence Comparisons

For the purposes of comparing two closely related polypeptide sequences, the “% sequence identity” between a first sequence and a second sequence may be calculated.

Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e. at least 85%, 86%, 87%, 88%, 89%, 90%, 91%, 92%, 93%, 94%, 95%, 96%, 97%, 98% or 99% sequence identity over a specified region), when compared and aligned for maximum correspondence over a comparison window (segment of sequence designated to be compared). Suitably, the comparison is performed over a window corresponding to the entire length of the reference sequence. Methods of alignment of sequences for comparison are well-known in the art, for example the BLAST algorithm also performs a statistical analysis of the similarity between two sequences (see, e.g., Karlin & Altschul, 1993, Proc. Nat'l. Acad. Sci. USA 90:5873-5787). Such procedures serve to compare a first, reference sequence to a second, comparison sequence, to determine the degree of identity or the number of additions, substitutions and/or deletions which when made to the first sequence would produce the second sequence.

### Sequence Variants

The term “amino acid” refers to any one of the naturally occurring amino acids, as well as amino acid analogues and amino acid mimetics that function in a manner which is similar to the naturally occurring amino acids. In an embodiment, the amino acid sequence of any of a polypeptide chain of the bispecific polypeptide molecule or antigen-binding domain or hinge domain subunit or Fc domain subunit or portion thereof may be artificial. Without limitation, and as discussed herein above, the polypeptide chain of the bispecific polypeptide molecule or antigen-binding domain or hinge domain subunit or Fc domain subunit or portion thereof may comprise at least one mutation, e.g. one or more mutation.

In an embodiment, at least one amino acid is substituted, added or deleted relative to the wildtype or native sequence provided herein. In the context of the binding domains, the at least one amino acid is (are) preferably located in a framework region (FR), a CDR or optionally a constant region, particularly in a framework region or a constant region, especially in a framework region, more particularly, any and all additions, substitutions and deletions of amino acids are in a framework region or a constant region, particularly in a constant region, alternatively the at least one amino acid is not located in any CDR.

Variations in sequence of the bispecific polypeptide molecule polypeptide chain or antigen-binding domain or hinge domain subunit or Fc domain subunit or portion thereof can be in the form of amino acid additions, substitutions and deletions, especially substitutions. For example, additions can be at the N and/or C termini of sequences and deletions can be at the N and/or C termini of sequences. Substitutions, i.e. substitution of one amino acid for another at a particular position in a sequence, are suitably conservative substitutions. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins 1984).

Suitably, sequence variations do not significantly adversely affect the ability of the antigen binding domain or antigen binding site to bind to its target MHC presented antigen or epitope, presented on the surface of the cancer cell or tumour cell or the cell surface antigen presented by the immune effector cell, for example the binding affinity of the variant is preferably 75% or more e.g. 80% or more e.g. 85% or more e.g. 90% or more e.g. 95% or more e.g. 98% or more e.g. 99% or more of that of the native antigen binding domain or antigen binding site. For the purpose of altering activity, for example to obtain a reduced binding to an Fc receptor, replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis or chemical modification. Various designations may be used herein to indicate the same amino acid mutation.

Where sequence variants are said to have at least 85% identity to a reference sequence, they may have at least 86%, 87%, 88%, 89%, 90%, 91%, 92%, 93%, 94%, 95%, 96%, 97%, 98% or 99% sequence identity to the relevant sequence. By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs for alignment.

### Polynucleotides

According to a further aspect of the invention there is provided a polynucleotide encoding the bispecific polypeptide molecule (bispecific) according to the invention.

In an embodiment, such a polynucleotide may be a chimeric polynucleotide comprising a gene encoding the bispecific polypeptide molecule and a heterologous promoter and/or other transcription control element such as a terminating signal operably linked thereto.

Since a complete bispecific polypeptide molecule comprises two polypeptide chains, two polynucleotides each encoding a chain of the bispecific may be provided or a polynucleotide encoding both chains of the bispecific may be provided. Yet further, the two chains of the bispecific may be linked by a cleavable peptide linker (e.g. a linker that cleaves in a host cell, for example a “self-cleaving” viral 2A sequence) and the polynucleotide may encode both chains of the bispecific and the linker such as nucleic acids.

The terms “nucleic acid” and “polynucleotide” are used interchangeably herein and refer to a polymeric macromolecule made from nucleotide monomers particularly deoxyribonucleotide or ribonucleotide monomers. The term encompasses polynucleotides containing known nucleotide analogues or modified backbone residues or linkages, which are naturally occurring and non-naturally occurring, which have similar properties as the reference polynucleotide, and which are intended to be metabolized in a manner similar to the reference nucleotides or are intended to have extended half-life in the system. Examples of such analogues include, without limitation, phosphorothioates, phosphoramidates, methyl phosphonates, chiral-methyl phosphonates, 2-O-methyl ribonucleotides, peptide-nucleic acids (PNAs). Suitably the term “polynucleotide” refers to naturally occurring polymers of deoxyribonucleotide or ribonucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature (e.g., in the case of cDNA). In an embodiment the polynucleotide of the invention is an artificial polynucleotide sequence (e.g., a cDNA sequence or polynucleotide sequence with non-naturally occurring codon usage). In one embodiment, the polynucleotides of the invention are DNA. Alternatively, the polynucleotides of the invention are RNA.

DNA (deoxyribonucleic acid) and RNA (ribonucleic acid) refer to polynucleotides having a backbone of sugar moieties which are deoxyribosyl and ribosyl moieties respectively. As used herein, a “corresponding RNA” is an RNA having the same sequence as a reference DNA but for the substitution of thymine (T) in the DNA with uracil (U) in the RNA.

In an embodiment the polynucleotide of the invention is a DNA, including single- or double-stranded DNA and straight-chain or circular DNA (i.e. plasmid DNA). Due to the degeneracy of the genetic code, a large number of different, but functionally identical polynucleotides can encode any given polypeptide. Such polynucleotide variations lead to “silent” (sometimes referred to as “degenerate” or “synonymous”) variants, which are one species of conservatively modified variations. Every polynucleotide sequence disclosed herein which encodes a polypeptide also enables every possible silent variation of the polynucleotide. Accordingly, each silent variation of a polynucleotide that encodes a polypeptide is implicit in each described sequence and is provided as an aspect of the invention.

Codons of the polynucleotide sequences of the invention may be altered in order that sequence variants of the bispecific or the antigen binding domains, hinge domain subunits or Fc domain subunits are expressed as discussed above. In an embodiment, up to 5 codons are altered, e.g., one, two, three, or four, e.g., one or two e.g., one codons are altered such that a different amino acid is encoded where the codon alteration occurs. For antigen binding domains codons encoding residues of the CDRs may be altered alternatively they are not altered. In an embodiment, the polynucleotides of the invention are codon optimised for expression in a host cell, particularly, a human host cell. The polynucleotide may suitably encode an immature bispecific polypeptide molecule or first and/or second polypeptide chain thereof which comprises an N-terminal leader sequence which is removed post translationally by cellular peptidases (such as signal peptidase) to produce the mature form.

### Vectors

According to a yet further aspect of the invention there is provided a vector encoding the bispecific polypeptide molecule (bispecific) of the invention. Specifically, there is provided a vector for delivery of a polynucleotide of the invention to cells or host cells comprising a polynucleotide encoding the bispecific of the invention.

As noted above, since the bispecific polypeptide molecule comprises two chains, two vectors each comprising a polynucleotide encoding a separate chain of the bispecific may be provided or a vector comprising a polynucleotide encoding both polypeptide chains of the bispecific may be provided. Yet further, the two chains of the bispecific may be linked by a cleavable peptide linker (e.g. a linker that cleaves in a host cell, particularly a permitting the encoded bispecific chains to associate) and the vector may comprise a polynucleotide which encodes both chains of the bispecific polypeptide molecule and the linker.

The, or each vector should suitably comprise such elements as are necessary for permitting transcription of a translationally active RNA molecule in the host cell, such as a promoter and/or other transcription control elements such as an internal ribosome entry site (IRES) or a termination signal. A “translationally active RNA molecule” is an RNA molecule capable of being translated into a protein by the host cell's translation apparatus.

Example promoters to drive transcription of the TCR chains include constitutive promoters such as the cytomegalovirus (CMV) promoter and elongation factor 1α (EF1α) promoter.

The vector may be, for example, a viral vector such as a lentiviral vector. Other examples of viral vectors include vectors derived from gamma-retrovirus, adenovirus, adeno-associated virus (AAV), alphavirus, herpes virus, arenavirus, measles virus, poxvirus or rhabdovirus. DNA molecules, for example transposons, may also be suitable vectors to transduce host cells.

### Host Cells and Production of Bispecifics

According to a further aspect of the present invention there is provided a host cell comprising the isolated polynucleotide of the invention or the vector/expression vector of the invention. The host cell may be a bacterial cell including for example E. coli or Bacillus subtilis or may be a yeast cell, for example Saccharomyces cerevisiae or Aspergillus, preferably said host cell is a mammalian host cell, for example a mammalian host cell selected from any one of: CHO cells, BHK cells, NSO cells, SP2/0 cells, YO myeloma cells, P3X63 mouse myeloma cells, PER cells, PER.C6 cells or hybridoma cells, and cells comprised within a transgenic animal or animal tissue.

Bispecifics of the invention can be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides followed by expression in a suitable organism or host cell to produce polypeptides. A gene encoding a bispecific polypeptide molecule of the invention can be synthetically produced by, for example, solid-phase DNA synthesis. In the context of this invention, one skilled in the art would understand that the polynucleotide sequences encoding the bispecific polypeptide molecule described in this invention could be readily used in a variety of protein production systems, including, for example, viral vectors.

For the purposes of production of polypeptides of the invention in a microbiological host (e.g., bacterial or fungal), polynucleotides of the invention will comprise suitable regulatory and control sequences (including promoters, termination signals etc) and sequences to promote polypeptide secretion suitable for protein production in the host.

Similarly, polypeptides of the invention could be produced by transducing cultures of eukaryotic cells with polynucleotides of the invention which have been combined with suitable regulatory and control sequences (including promoters, termination signals etc) and sequences to promote polypeptide secretion suitable for protein production in these cells.

Improved isolation of the polypeptides of the invention produced by recombinant means may optionally be facilitated through the addition of a purification tag at one end of the polypeptide.

### Compositions

The invention provides a pharmaceutical composition comprising the bispecific polypeptide molecule, polynucleotide, vector, or host cell of the invention and a pharmaceutically acceptable carrier. Suitably the pharmaceutical composition is formulated under sterile conditions and is suitable for parenteral administration. For parenteral administration, the carrier preferably comprises water and may contain buffers for pH control, stabilising agents e.g., surfactants and amino acids and tonicity modifying agents, e.g., salts and sugars.

### Use as a Medicament, Method of Treatment, Cancer Treatment

The present invention further provides the bispecific polypeptide molecule, polynucleotide, vector, host cell or pharmaceutical composition of the invention for use as a medicament.

Accordingly, there is provided a method of treatment of cancer comprising administering to a patient in need thereof the bispecific polypeptide molecule, polynucleotide, vector, host cell or pharmaceutical composition of the invention.

According to a further aspect of the invention, cells which have been obtained from a patient may modified ex vivo (e.g., by transduction), optionally ex vivo, by the introduction of a heterologous polynucleotide of the invention or by a vector of the invention so that they express bispecific polypeptide molecule according to the invention. Optionally the modified cells are immune effector cells, for example T-cells.

Hence in one aspect there is provided an ex vivo process comprising (i) obtaining cells, particularly immune effector cells from a patient, (ii) optionally expanding the cells (iii) introducing a heterologous polynucleotide according to the invention or a vector according to the invention into the cells, particularly immune effector cells to produce modified cells, particularly effector immune cells which express a bispecific polypeptide molecule according to the invention; and (iii) reintroducing said modified cells, particularly immune effector cells into the patient.

Hence the present invention provides a method for the treatment of cancer comprising administering to a patient in need thereof transduced cells, preferably immune effector cells, which have been obtained from said patient which modified ex vivo by the introduction of a heterologous polynucleotide of the invention or by a vector of the invention so that they express bispecific polypeptide molecule.

There is also provided use of the aforementioned modified (e.g. transduced) cells, in the manufacture of a medicament for the treatment of cancer. In the foregoing methods of treatment, the cancer treated is preferably selected those cancers set out in the section on cancer treatment below.

According to a further aspect of the invention there is provided a method of treating cancer comprising administering said bispecific polypeptide molecule, polynucleotide, vector, host cell or pharmaceutical composition of the invention to an individual to be treated.

There is also provided said bispecific polypeptide molecule, polynucleotide, vector, host cell or pharmaceutical composition of the invention for use in the treatment of cancer.

There is also provided the use of said bispecific polypeptide molecule, polynucleotide, vector, host cell or pharmaceutical composition of the invention in the manufacture of a medicament to treat cancer.

Also, there is provided a method of treating cancer in a subject comprising administering a therapeutically effective amount of bispecific polypeptide molecule, polynucleotide, vector, host cell or pharmaceutical composition of the invention to the subject.

According to the invention the cancer may include solid tumours and blood cancers, for example cancers selected from blood (e.g. leukemia), bladder, lung, colorectal, prostate, stomach, liver, cervix, uterine, oesophagus, thyroid, lymphoma, bladder, non-hodgkin lymphoma, pancreas, leukaemia, kidney, corpus uteri, lip, oral cavity, melanoma of skin, ovary, brain, central nervous system, larynx, multiple myeloma, nasopharynx, gallbladder, oropharynx, hypopharynx, hodgkin lymphoma, testis, thyroid, salivary glands, vulva, penis, kaposi sarcoma, breast, mesothelioma, vagina cancer, in each case including metastatic cancer. Equally well, it is also envisaged that all embodiments of the present invention that may be used to treat cancer may potentially be used to prevent cancer. Thus, corresponding methods and uses and substances for use to prevent cancer are provided as an aspect of the invention.

### Combinations

According to a yet further aspect of the invention there is provided a combination therapeutic for the treatment of cancer comprising:
a) the bispecific polypeptide molecule, polynucleotide, vector, host cell or pharmaceutical composition of the invention in combination with
b) a further cancer therapeutic agent.

Accordingly, the bispecific polypeptide molecule, polynucleotide, vector, host cell or pharmaceutical composition of the invention may be administered separately, simultaneously or sequentially with an anti-cancer agent.

More generally, there is provided a pharmaceutical composition comprising:
a) the bispecific polypeptide molecule, polynucleotide, vector, host cell or pharmaceutical composition of the invention; and
b) an anti-cancer agent.

Accordingly, the bispecific polypeptide molecule, polynucleotide, vector, host cell or pharmaceutical composition of the invention may be administered separately, simultaneously or sequentially with an anti-cancer agent.

Further anti-cancer agents that may be included in a combination therapy include immune check point inhibitors e.g. selected from PD-1 inhibitors, such as pembrolizumab, (Keytruda) and nivolumab (Opdivo), PD-L1 inhibitors, such as atezolizumab (Tecentriq), avelumab (Bavencio) and durvalumab (Imfinzi) and CTLA-4 inhibitors such as ipilimumab (Yervoy), other immune stimulants such as interferons (e.g. interferon a, 3 or y), steroids e.g. prednisolone and alkylating agents such as platinum-based anti-neoplastic agents e.g. cisplatin, carboplatin and oxaliplatin.

Other features of the present invention will become apparent from the following examples. Unless stated otherwise, any feature disclosed herein may be replaced by an alternative feature serving the same or a similar purpose.

### STATEMENT CLAUSES OF INVENTION

1. A bispecific polypeptide molecule capable of simultaneously binding a first and second antigen, wherein the bispecific polypeptide molecule comprises a first polypeptide chain and a second polypeptide chain, wherein:
(A) the first polypeptide chain comprises: (i) a first antigen-binding domain, (ii) a first hinge domain subunit, (iii) a first Fc domain subunit; and
(B) the second polypeptide chain comprises: (i) a second antigen-binding domain, ii) a second hinge domain subunit, (iii) a second Fc domain subunit;
wherein the first and second hinge domain subunits are capable of forming a stable association as a hinge domain and the first and second Fc domain subunits are capable of forming a stable association as an Fc domain or Fc domain portion, such that the two polypeptide chains are connected by covalent and/or non-covalent bonds between the hinge domain subunits and Fc-domain subunits, preferably wherein the first and second antigens are expressed on two distinct cells.
2. The bispecific polypeptide molecule according to clause 1 wherein the first antigen-binding domain comprises a first binding region of a variable domain (VD1) of an antibody and a second binding region of a variable domain (VD2) of an antibody and a first linker (LNK1) connecting said domains wherein the first binding region (VD1) and the second binding region (VD2) associate to form a first antigen binding site (VD1)(VD2).
3. The bispecific polypeptide molecule according to clause 2, wherein VD1 is N-terminal to VD2 or wherein VD2 is N terminal to VD1, and wherein VD1 and VD2 are connected by the first linker LNK1, optionally wherein VD1 is N-terminal to VD2 and VD1 and VD2 are connected by LNK1.
4. The bispecific polypeptide molecule according to either clause 2 or clause 3, wherein the first binding region of a variable domain (VD1) comprises an antibody variable light domain (VL) or epitope binding portion thereof and the second binding region of a variable domain (VD2) comprises an antibody variable heavy domain (VH) or antigen binding portion thereof, optionally wherein said variable light domain (VL) and/or variable heavy domain (VH) epitope or respective binding portion thereof, may further comprise part or all of a respective antibody heavy chain constant domain, CL and/or CH1.
5. The bispecific polypeptide molecule according to any one of clauses 2 to 4, wherein the linker LNK1 (a) is a flexible linker of between 3 and 20 amino acids and/or comprising of small, non-polar and/or small polar amino acids, optionally wherein LNK1 (a) comprises amino acids including glycine, or glycine serine and/or threonine or (b) comprises at least one sequence motif selected from GGGS, GGGGS, TVLRT, TVSSAS, and TVLSSAS or (c) comprises a sequence selected from GGGGSGGGGSGGGGSGGGGS, (SEQ ID NO.46) or GSADDAKKDAAKKDGKS, (SEQ ID NO.47).
6. The bispecific polypeptide molecule according to clause 5 wherein the linker LNK1 comprises all or part of an immunoglobulin (Ig) hinge sequence.
7. The bispecific polypeptide molecule according to any of clauses 2 to 6 wherein VD1 and VD2 comprise an engineered disulphide bridge introducing a covalent bond between VD1 and VD2, wherein cysteines are introduced into framework region 4 (FR4) in case of VL and framework region 2 (FR2) in case of VH or are introduced into framework region 4 (FR4) in case of VH and framework region 2 (FR2) in case of VL.
8. The bispecific polypeptide molecule according to clause 7, wherein the linker LNK1 comprises a first LNK1 cysteine residue (Cys) and a second LNK1 cysteine residue (Cys), wherein a first disulphide bond is formed between the introduced VH cysteine (Cys) and a first LNK1 Cys and/or a second disulphide bond is formed between the introduced VL Cys and a second LNK1 Cys.
9. The bispecific polypeptide molecule according to clause 8, wherein the linker LNK1 comprises the sequence CPPC (SEQ ID No.52) and/or the LNK1 comprises an amino acid sequence selected from any of: GGGSGGSGGCPPCGGSGG (SEQ ID NO.17), GGGSDDSGGCPPCGGKGG (SEQ ID NO.18), and GGAAGGSGGCPPCGGSGG (SEQ ID NO.19).
10. The bispecific polypeptide molecule according to any of clauses 1 to 9, wherein the first antigen-binding domain comprises or consists of a single chain Fv (scFv).
11. The bispecific polypeptide molecule according to any preceding clause, wherein the first antigen-binding domain is capable of specifically binding to a cell surface antigen expressed on the surface of an immune effector cell, optionally human immune effector cell, optionally wherein said immune effector cell expresses an activating receptor and wherein the first antigen-binding domain binds to the activating receptor resulting in immune effector cell activation.
12. The bispecific polypeptide molecule according to clause 11 wherein the immune effector cell is a T-cell, a CD4+ T-cell, a CD8+ T-cell, a natural killer cell, a macrophage, a granulocyte, or a dendritic cell, optionally a CD8+ T-cell.
13. The bispecific polypeptide molecule according to clause 11 or 12, wherein activating receptor is selected from the group consisting of: CD3, such as the CD3y, CD35, and CD3E chains, CD4, CD7, CD8, CD10, CD11 b, CD11 c, CD14, CD16, CD18, CD22, CD25, CD28, CD32a, CD32b, CD33, CD41, CD41 b, CD42a, CD42b, CD44, CD45RA, CD49, CD55, CD56, CD61, CD64, CD68, CD94, CD90, CD1 17, CD123, CD125, CD134, CD137, CD152, CD163, CD193, CD203c, CD235a, CD278, CD279, CD287, Nkp46, NKG2D, GITR, FCERI, TCRa/3 and TCRy/5, HLA-DR.
14. The bispecific polypeptide molecule according to any of clauses 1 to 13, wherein the first binding region of a variable domain (VD1) and the second binding region of a variable domain (VD2) are derived from the humanised anti-CD3 antibody or variant thereof.
15. The bispecific polypeptide molecule according to clause 14, wherein the humanised anti-CD3 antibody variant is a humanised anti-CD3 antibody variant, UCHT1, optionally UCHT1 v.9, wherein the variable domain (VD1) comprises an antibody variable light domain (VL) of SEQ ID No.1. or epitope binding portion thereof and wherein the second binding region of a variable domain (VD2) comprises an antibody variable heavy domain (VH) of SEQ ID No.2, or epitope binding portion thereof.
16. The bispecific polypeptide molecule according to any of clauses 1 to 15, wherein said first antigen binding domain or said first antigen binding site (VD1)(VD2) binds or specifically binds the first antigen with an affinity (KD) of about 100 pM or less.
17. The bispecific polypeptide molecule according to any preceding clause, wherein the second antigen-binding domain comprises a first binding region of a variable domain (VR1) of a TCR and a second binding region of a variable domain (VR2) of a TCR and a second linker (LNK2) connecting said domains wherein the first binding region (VR1) and the second binding region (VR2) associate to form a second antigen binding site (VR1)(VR2).
18. The bispecific polypeptide molecule according to clause 17 wherein the first binding region of a variable domain (VR1) comprises a TCR α chain variable domain (Vα) or antigen or MHC-associated peptide epitope binding portion thereof and the second binding region of a variable domain (VR2) comprises a TCR β chain variable domain (Vβ) or antigen or MHC-associated peptide epitope binding portion thereof, optionally wherein the first binding region of a variable domain (VR1) may further comprise part or all of a TCR α chain constant domain, Cα, preferably linked to or fused to the C-terminus of the Vα domain and/or the second binding region of a variable domain (VR2) may further comprise part or all of TCR β chain constant domain, Cβ, preferably linked to or fused to the C-terminus of the Vβ domain.
19. The bispecific polypeptide molecule according to clause 18, wherein VR1 is N-terminal to VR2 or VR2 is N terminal to VR1; and wherein VR1 and VR2 are connected by LNK2, optionally wherein VR2 is N-terminal to VR1 and VR2 and VR1 are connected by LNK2.
20. The bispecific polypeptide molecule according to any one of clauses 17 to 19, wherein the linker LNK2 (a) is a flexible linker of between 3 and 20 amino acids and/or comprising of small, non-polar and/or small polar amino acids, optionally wherein LNK2 (a) comprises amino acids including glycine, or glycine serine and/or threonine or (b) comprises at least one sequence motif selected from GGGS, GGGGS, TVLRT, TVSSAS, and TVLSSAS or (c) comprises a sequence selected from GGGGSGGGGSGGGGSGGGGS, (SEQ ID NO.46) or GSADDAKKDAAKKDGKS, (SEQ ID NO.47).
21. The bispecific polypeptide molecule according to clause 20 wherein the linker LNK2 comprises all or part of an immunoglobulin (Ig) hinge sequence.
22. The bispecific polypeptide molecule according to any of clauses 17 to 21 wherein VR1 and VR2 comprise an engineered disulphide bridge introducing a covalent bond between VR1 and VR2, wherein the cysteines are introduced into framework region 4 (FR4) in case of Vα and framework region 2 (FR2) in case of V β or are introduced into framework region 4 (FR4) in case of V β and framework region 2 (FR2) in case of Vα.
23. The bispecific polypeptide molecule according to clause 22, wherein the linker LNK2 comprises a first LNK2 cysteine (Cys) and a second LNK2 cysteine (Cys), wherein a first disulphide bond is formed between the introduced Vβ cysteine (Cys) and a first LNK2 Cys and/or a second disulphide bond is formed between the introduced Vα Cys and a second LNK2 Cys, optionally wherein the linker LNK2 comprises the sequence CPPC (SEQ ID No.52).
24. The bispecific polypeptide molecule according to clause 22 or 23, wherein the LNK2 comprises an amino acid sequence selected from any of: GGGSGGSGGCPPCGGSGG (SEQ ID NO.17), GGGSDDSGGCPPCGGKGG (SEQ ID NO.18), and GGAAGGSGGCPPCGGSGG (SEQ ID NO.19).
25. The bispecific polypeptide molecule according to any preceding clause, wherein the second antigen binding domain comprises or consists of a single chain TCR variable domain (scTv).
26. The bispecific polypeptide molecule according to any preceding clause, wherein the second antigen binding domain or said second antigen binding site (VR1)(VR2) binds or specifically binds the second antigen with an affinity (KD) of about 100 μM or less.
27. The bispecific polypeptide molecule according to clause 26, wherein the second antigen is an MHC-associated peptide epitope or antigen (pMHC).
28. The bispecific polypeptide molecule according to clause 26 or clause 27, wherein the second antigen is a cancer or tumour cell antigen and/or is detectibly present on the cell surface or a cancer or tumour cell and/or the MHC-associated peptide epitope or antigen is a cancer or tumour cell epitope or antigen and/or is detectibly present on the cell surface or a cancer or tumour cell.
29. The bispecific polypeptide molecule according to any one of clauses 26 to 28, wherein the binding to said second antigen or MHC-associated peptide epitope or antigen, results in immune effector cell activation.
30. The bispecific polypeptide molecule according to any one of clauses 26 to 29 wherein the antigen is any one of PRAME, HIV-GAG, HIV-TAX, or MAGE-A4, antigen or a peptide epitope thereof.
31. The bispecific polypeptide molecule according to any one of clauses 1 to 30 wherein the hinge domain comprises a first hinge domain subunit and a second hinge domain subunit, wherein the first hinge domain subunit is fused to the first antigen-binding domain and the second hinge domain subunit is fused to the second antigen-binding domain, wherein the fusion orientates the antigen-binding domain at the N-terminal and the hinge domain subunit at the C-terminal and wherein said fusion is by means of a peptide bond between the c-terminal amino acid of the respective antigen binding domain and the N-terminal amino acid of hinge domain subunit.
32. The bispecific polypeptide molecule according to clause 31, wherein said first hinge domain subunit and said second hinge domain subunit is between 2 and 25 amino acids in length and comprises between 1 and 4 cysteine residues, optionally wherein the hinge domain comprises two identical hinge domain subunits.
33. The bispecific polypeptide molecule according to any one of clauses 1 to 32 wherein the hinge domain:
(a) is an antibody hinge domain, which is a (human) IgG hinge domain selected from IgG1, 2 or4 or portion thereof, optionally wherein the hinge domain subunit comprises any one of the sequences of SEQ ID NO.20, 21, 22, 23 or 24 or variant sequence thereof, or
(b) comprises a hinge domain subunit comprising SEQ ID NO.80, EPKSSDKTHTCPPCPAPEAAGG or variant sequence thereof or an N-terminal truncation thereof such that any of 1, 2, 3, 4, 5, 6, 7, 8, 9 or 10 of the N-terminal amino acid residues are deleted, or
(c) comprises a hinge domain subunit comprising any one of the sequences of SEQ ID NO.78, 79 or 80.
34. The bispecific polypeptide molecule according to any one of clauses 31 to 33 wherein the hinge domain provides a connecting sequence (CNX), wherein said connecting sequence (CNX) comprises a first cysteine residue connected to a second cysteine residue by a disulphide bond (i.e. Cys=Cys, SEQ ID NO.175) and wherein either (i) the first cysteine residue is connected to the first antigen binding domain and the second cysteine residue is connected to the second antigen binding domain, optionally by means of a peptide bond, or (ii) the first cysteine is peptide bonded to a first intervening polypeptide sequence comprising of any one of 1, 2, 3, 4, 5, 6, 7, 8, 9, or 10 amino acid residues which intervene the first cysteine residue and the first antigen binding domain or C-terminal amino acid of the first antigen binding domain; and/or the second cysteine is peptide bonded to a second intervening polypeptide sequence comprising of any one of 1, 2, 3, 4, 5, 6, 7, 8, 9, or 10 amino acid residues which intervene the second cysteine and the second antigen binding domain or C-terminal amino acid of the second antigen binding domain, optionally the length of the first and second intervening polypeptide sequences are the same.
35. The bispecific polypeptide molecule according to clause 34 wherein said first cysteine residue is provided by the first hinge domain subunit of the hinge domain and the second cysteine is provided by the second hinge domain subunit of the hinge domain.
36. The bispecific polypeptide molecule according to either clause 34 or 35, wherein said connecting sequence (CNX) connects the first antigen binding domain to the second antigen binding domain, such that the first antigen binding domain and the second antigen binding domain may simultaneously bind a first antigen and second antigen respectively, and wherein said simultaneous binding provides immune effector cell activation.
37. The bispecific polypeptide molecule according to according to any one of clauses 34 to 36 wherein the first antigen binding domain is connected to the second antigen binding domain by a connection sequence or connector sequence (CNX), such that the C-terminal end of the first antigen binding domain is separated from the C-terminal end of the second antigen binding domain by any of 2, 3, 4, 5, 6, 7, 8, 9, 10, 11, 12, 13, 14, 15, 16, 17, 18, 19, 20, 21, 22, 23 or 24 peptide bonds in combination with 1 disulphide bond (e.g. cys=cys).
38. The bispecific polypeptide molecule according to any one of clauses 34 to 37, wherein the CNX connects the first antigen binding domain to the second antigen binding domain, such that the distance between the first antigen binding domain and the second antigen binding domain permits the first antigen binding domain and the second antigen binding domain to simultaneously bind a first antigen and second antigen respectively, and wherein said simultaneous binding provides immune effector cell activation and/or provides immune effector cell target cell killing, optionally wherein said target cell is a cell presenting a cancer or tumour cell antigen, for example a cancer or tumour cell.
39. The bispecific polypeptide molecule according to clause 38, wherein said distance is any of about 1.36, 1.74, 2.12, 2.5, 2.88, 3.26, 3.64, 4.02, 4.4, 4.78, 5.16, 5.54, 5.92, 6.3, 6.68, 7.06, 7.44, 7.82, 8.2, 8.58, 8.96, 9.34 or about 9.72 nm, said distances in nm being the sum of alpha-carbon to alpha-carbon distances plus the distance of between the two alpha carbons of the first and second cysteine residues.
40. The bispecific polypeptide molecule according to any previous clause, wherein the Fc domain is derived from human IgG1, IgG2 or IgG4 Fc domain or portion thereof, or a chimera of any two or three of an IgG1, IgG2 or IgG4 Fc domain or portion thereof, preferably wherein the portion is a dimerising portion.
41. The bispecific polypeptide molecule according to clause 40, wherein the Fc domain exhibits reduced binding affinity to an Fc receptor and/or reduced effector function, as compared to a native IgG1, IgG2 or IgG4 Fc domain and/or wherein the Fc domain comprises one or more amino acid substitution that reduces binding to an Fc receptor and/or effector function as compared to a native IgG1, IgG2 or IgG4 Fc domain.
42. The bispecific polypeptide molecule according to any one of clauses 40 to 41, wherein the Fc domain comprises at least one effector function silencing mutation at a residue selected from positions 233, 234, 235, 236, 297 and 331 and/or wherein said effector function silencing mutation is generated by replacing at least one residue in position 233, 234, 235, 236, and 331 (EU numbering).
43. The bispecific polypeptide molecule according to any of clauses 40 to 42, wherein in the CH3 domain of a first Fc domain subunit or portion thereof comprises a sequence in which an amino acid residue is replaced with an amino acid residue having a larger side chain volume, thereby generating a protuberance within the CH3 domain of the said subunit which is positionable in a cavity within the CH3 domain of a second Fc domain subunit or portion thereof, and which in the CH3 domain of the said Fc domain subunit or portion thereof an amino acid residue is replaced with an amino acid residue having a smaller side chain volume, thereby generating a cavity within the CH3 domain of the subunit within which the protuberance within the CH3 domain of the first subunit is positionable, preferably wherein the formation of heterodimers is facilitated.
44. The bispecific polypeptide molecule according to clause 43, wherein the Fc domain comprises a CH3 domain comprising at least one mutation that facilitates the formation of heterodimers, for example, wherein the mutations are located at any position selected from 366, 368, 405, and 407, preferably, wherein said mutations comprise T366W and T366'S, L368A′ and Y407V as knob-into-hole mutations (EU numbering) and/or wherein the Fc domain comprises CH2 and CH3 domain(s) comprising at least two additional cysteine residues, for example S354C and Y349C or L242C and K334C (EU numbering).
45. The bispecific polypeptide molecule according to any previous clause, wherein the bispecific polypeptide molecule comprises:
(A) a first polypeptide chain comprising a first binding domain comprising (i) a first binding region of a variable domain (VD1) comprising any of one, two or three CDR sequences selected from: SEQ ID NO.3 RASQDIRNYLN, SEQ ID NO.4 YYTSRLES, and SEQ ID NO.5 QQGNTLPWT, or variants thereof; and (ii) a second binding region of a variable domain (VD2) comprising any of one, two or three CDR sequences selected from: SEQ ID NO.6 GYTMN, SEQ ID NO.7 LINPYKGVSTYNQKFKD, and SEQ ID NO.8 SGYYGDSDWYFDV, or variants thereof; preferably wherein the variant CDR respectively has 1, 2, or 3 amino acid variations selected from additions, substitutions and deletions with respect to the recited native sequence; and
(B) a second polypeptide chain comprising a second binding domain comprising any of:
a. (i) a first binding region of a variable domain (VR1) which comprises any of one, two or three CDR sequences selected from: SEQ ID NO.11 DRGSQS, SEQ ID NO.12 IYSNGD, and SEQ ID NO.13 AAVIDNDQGGILT, or variants thereof and (ii) a second binding region of a variable domain (VR2) which comprises any of one, two or three CDR sequences selected from: SEQ ID NO.14 PGHRA, SEQ ID NO.15 YVHGEE, and SEQ ID NO.16 ASSPWDSPNVQY, or variants thereof;
b. (i) a first binding region of a variable domain (VR1) which comprises any of one, two or three CDR sequences selected from: SEQ ID NO.110, 111, 112 or variants thereof and (ii) a second binding region of a variable domain (VR2) which comprises any of one, two or three CDR sequences selected from: SEQ ID NO.113, 114 and 115, or variants thereof;
c. (i) a first binding region of a variable domain (VR1) which comprises any of one, two or three CDR sequences selected from: SEQ ID NO.116, 117, 118 or variants thereof and (ii) a second binding region of a variable domain (VR2) which comprises any of one, two or three CDR sequences selected from: SEQ ID NO.119, 120 and 121, or variants thereof; or
d. (i) a first binding region of a variable domain (VR1) which comprises any of one, two or three CDR sequences selected from: SEQ ID NO.122, 123, 124 or variants thereof and (ii) a second binding region of a variable domain (VR2) which comprises any of one, two or three CDR sequences selected from: SEQ ID NO.125, 126 and 127, or variants thereof; wherein the variant CDR respectively has 1, 2, or 3 amino acid variations selected from additions, substitutions and deletions with respect to the recited native sequence.
46. The bispecific polypeptide molecule according to any previous clause, wherein the bispecific polypeptide molecule comprises a first polypeptide chain and a second polypeptide chain, wherein:
(a) the first polypeptide chain comprises the sequence SEQ ID No.29, and the second polypeptide chain comprises the sequence SEQ ID No.30 or SEQ ID 31,
(b) the first polypeptide chain comprises the sequence SEQ ID No.105, and the second polypeptide chain comprises the sequence SEQ ID No.83 or SEQ ID 84,
(c) the first polypeptide chain comprises the sequence SEQ ID No.106, and the second polypeptide chain comprises the sequence SEQ ID No.91,
(d) the first polypeptide chain comprises the sequence SEQ ID No.29, and the second polypeptide chain comprises any one of the sequences selected from SEQ ID No.85, 86, 87, 88, 89, and 90;
(e) the first polypeptide chain comprises the sequence SEQ ID No.29, and the second polypeptide chain comprises any one of the sequences selected from SEQ ID No.92, 93, 94, and 95;
(f) the first polypeptide chain comprises the sequence SEQ ID No.29, and the second polypeptide chain comprises any one of the sequences selected from SEQ ID No.96, 97, 98, 99, and 100;
(g) the first polypeptide chain comprises the sequence SEQ ID No.29, and the second polypeptide chain comprises any one of the sequences selected from SEQ ID No.101, 102, 103 or 104,
(h) the first polypeptide chain comprises the sequence SEQ ID No.29, and the second polypeptide chain comprises any one of the sequences selected from SEQ ID No.136, 137, 138, 139, 140, or
(i) the first polypeptide chain comprises the sequence SEQ ID No.29, and the second polypeptide chain comprises any one of the sequences selected from SEQ ID No.141, 142, 143, 144 or 105.
47. An isolated polynucleotide encoding bispecific polypeptide molecule according to any of clauses 1 to 46.
48. A polypeptide encoded by the isolated polynucleotide of clause 47.
49. A vector, particularly an expression vector, comprising the isolated polynucleotide of clause 47.
50. A host cell comprising the isolated polynucleotide of clause 47 or the expression vector of clause 49.
51. A method of producing the bispecific polypeptide molecule according to any of clauses 1 to 46 and 48, comprising the steps of a) culturing the host cell of clause 50 under conditions suitable for the expression of the bispecific polypeptide molecule and b) recovering the bispecific polypeptide molecule.
52. A bispecific polypeptide molecule produced by the method of clause 51.
53. A pharmaceutical composition comprising the bispecific polypeptide molecule according to any of clauses 1 to 46 and 48, or polynucleotide of clause 47 or vector of clause 49 or host cell of clause 50 and a pharmaceutically acceptable carrier.
54. The bispecific polypeptide molecule according to any of clauses 1 to 46 and 48 or the pharmaceutical composition of clause 53 for use as a medicament.
55. The bispecific polypeptide molecule according to any of clauses 1 to 46 and 48 or the pharmaceutical composition of clause 53 for use in the treatment of a disease in an individual in need thereof, wherein the disease is cancer.
56. A method of treatment of cancer comprising administering to a patient in need thereof bispecific polypeptide molecule according to any of clauses 1 to 46 and 48 or the pharmaceutical composition of clause 53.
57. The use of bispecific polypeptide molecule according to any of clauses 1 to 46 and 48 or the pharmaceutical composition of clause 53 in the manufacture of a medicament to treat cancer.

### EXAMPLES

### Example 1: Bispecific Engager Design

The inventors postulated that bispecific engagers that re-direct patient T cells to cancer cells via recognition of tumour antigens displayed on HLA class I molecules need to be designed to maintain the orientation of the HLA class I to permit it to bind the T cell's CD8a/b coreceptor. The inventors proposed that optimal designs should produce a conformation of pHLA-bispecific engager-CD3 on targeted T cell that matches the synapse produced by the interaction of the pHLA on a cancer cell (or other presenting cell) with the natural TCR receptor on the surface of a T cell.

(a) Bispecific engager conformation

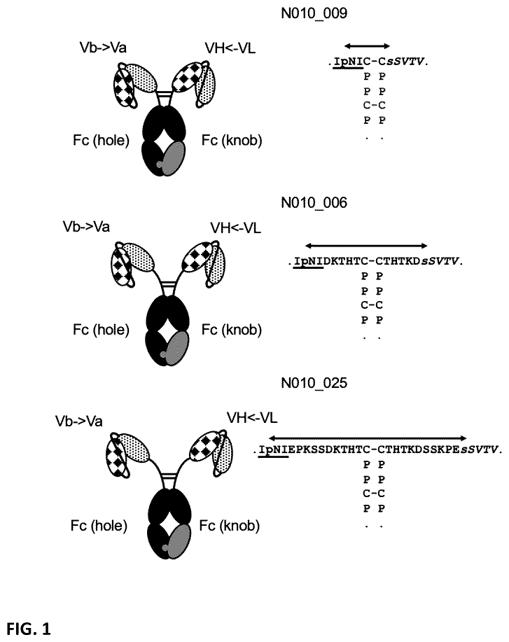

To test the proposed hypothesis that the size of the bispecific engager is important for ensuring efficacy, a panel of three TCR-directed immune cell engager (TCE) molecules were created using a PRAME epitope SLLQHLIGL (SEQ ID NO.81) HLA-A2-reactive scTv and an anti-CD3-reactive scFv (UCHT1v09, derived from Shalaby et al., J Exp Med. 1992 Jan. 1; 175(1):217-25), SEQ ID NO.1 and 2, held together by KiH Fc molecules containing different length-hinge regions on their paired Fc molecules (Carter. P., J Immunol Methods 2001; 248:7-15). Variants of the Fc domain were designed and incorporated into the produced TCE constructs by the inventors which comprised Fc silencing mutations. These mutations reduced or eliminated effector function while retaining binding to the neonatal Fc receptor. A number of mutations have been described to silence the Fc domain, including LALA (L234A, L235A), Xu D., et. al., Cell Immunol., 2000; 200:16-26, further mutations are described in the present description. FIG. 1 shows three TCE designs created by the inventors to demonstrate the role of distances between the antigen binding moieties and activity. The left side of FIG. 1 shows schematic diagrams of each TCE molecules, and the right side reveals the differences in the symmetrical Fc hinges that bind these two single-chain antigen binding moieties together. The portions of the TRAC chain and portions of the VH chain shown in the diagram (as well as the unshown portions of the TRAJ chain) also contribute to the distances between the antigen-binding moieties. Fc pairing was ensured by including Knob mutations (on the scFv chain) and Hole mutations on the scTv chains (using the KiH mutations described by Carter. P, J Immunol Methods 2001; 248:7-15).

The arrows shown in FIG. 1 define one of the key aspects of the invention, namely the importance of the distance between the scFv and scTv moieties. This can be expressed as a linear term defined by the sum of the distance between the C-terminal residues of the TRAJ and the VH peptides; using the metrics of polypeptide biology, these can be estimated as the sum of Calpha (CA) to CA distances (0.38 nm) plus one C—C distance derived from the distance between the CA of the Cys residues (0.6 nm) that span this distance. In the case of the 3 designs shown in FIG. 1, these estimates equal 2.1 nm (4×0.38+1×0.6) for N010_009, 5.9 nm (14×0.38+1×0.6) for N010_006, and 9.7 nm (24×0.38+1×0.6) for molecule N010_025.

### (b) Bispecific Stability can be Enhanced by Linker Crosslinking

Although scFvs have been developed into therapeutic molecules, several of the newer clinical candidates contain residues mutated to Cys in their VH/VL interfaces in framework2 and framework4 (FW2 & FW4) to permit the formation of direct S—S bonds between the VH and VL chains that can increase stability (Reiter et al., Nature Biotech., 1996; Bhatta & Humphreys, antibodies, 2018). One scTv-based bispecific engager (ABBV-184), which has been in clinical trials, also contains this type of direct Va-Vb S—S bond (Chervin et al., Mol. Cancer Ther., 2023 Aug. 1; 22(8):903-912), also located in these FW2/4 locations.

The inventors were motivated to explore the use of linkers containing a CPPC sequence derived from an IgG hinge as a means to crosslink the Cys residues (anchor residues) introduced within the interface between the Va and Vb moieties of an scTvs. The CPPC linkage was selected due to its known properties in mediating the stable linkage between two Fc domains of antibody molecules, which is ensured by the steric prevention of self-linkage by the intervening Pro residues. The application of such a feature to scTvs is challenging, due to the tremendous diversity of the TRAV sequences (over 40 highly diverse human isotypes, www.IMGT.org; which encode FW2) TRAJ (approximately 70 diverse human isotypes, IMGT.org; which encode FW4), TRBV (over 40 diverse human isotypes, IMGT.org; which encode FW2) TRBJ (16 diverse human isotypes, IMGT.org; which encode FW4).

Molecular modeling was employed to ascertain the best positioning of the cysteine anchor residues between the framework domains, with reference to the CPPC linkers to achieve the cross-connection the alpha and beta chains and which connect in the opposite orientations on the non-CDR side of the molecule.

To position these anchor residues on the TRBV-TRAJ orientation and the TRAV-TRBJ orientation, the inventors carefully reviewed the pdb database for model TCR structures which had been determined for full-length TCRs, as well employing deep learning computational models, ImmuneBuilder (Abanades, B., et al. ImmuneBuilder: Deep-Learning models for predicting the structures of immune proteins, Commun Biol, 6, 575, 2023) with TCR-specific model generating methods, TCRBuilder2, these were used to generate model structures of the scTvs and their wild types for the basis of anchor positioning.

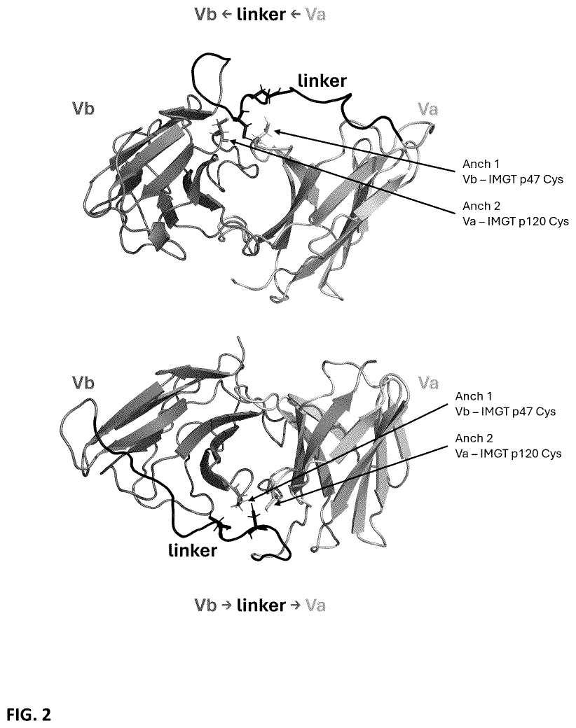

Based on these analyses, the technical value of linkers that bridged IMGT position Vα p48 to the first C of the CPPC-containing linker and the second C of the linker to IMGT position Vb p120 in a Va-Vb orientation scTv and linkers that bridged IMGT position Vb p47 to the first C of the CPPC-containing linker and the second C of the linker to IMGT position Vα p120 were further explored by modeling them onto the public structure (Protein Data Bank {pdb}coordinate file number 5e9d) of the RD1-MART1^{HIGH}scTv (Harris, D. T., et. al., (2016) Structure 24: 1142-1154). FIG. 2a shows a candidate linker inserted between the C-termini and N-termini of sequentially expressed Va/Vb polypeptides found in the modelled 5e9d sequence. These designs included the short portions of the TRAC and TRBV sequences found in the 5e9d sequence, and for the Vb->Va orientation, the first residue of the Va chain was mutated to a Gly residue. FIGS. 2b and 2c are images of structures created from 5e9d by using PyMol and subjected to energy minimization in GROMACS (www.gromacs.org) software for molecular dynamics simulation. Energy minimization was performed with steepest descent minimization algorithm to 100,000 steps with maximum force limit 100.00 KJ/mol/nm at 0.001 ps step size with PME treatment of long-range electrostatic interactions.

As can be seen in FIG. 2b and FIG. 2c, the anchors inserted into these models fit well with the CPPC-containing linker. In these designs (as in others throughout the present description) the N-termini of the TRAV and TRBV as well as the C-terminal ends of the TRAJ/TRBJ and the TRAC/TRBC fragments can be considered part of the linking moiety.

Based on this modelling success, 5 different CPPC-linked scTvs were created using the same sequence parameters used for our initial model. These designs are shown in Table 1, with various sequence data, including proposed anchor positions (with their IMGT numbers), and their SEQ ID numbers. In addition, TCRBuilder was used to construct models of each of these scTvs (without a linker), and the alpha carbon to alpha carbon (CA-CA) distances between the chosen positions of the anchor residues are also shown in Table 1.

Many of the designs in Table 1 share a single linker (GGGSGGSGGCPPCGGSGG), SEQ ID NO.17, but two have one alternative linker design (GGAAGGSGGCPPCGGSGG), SEQ ID NO.19, and two have a second alternative linker design (GGGSDDSGGCPPCGGKGG), SEQ ID NO.18. These later two designs were explored due to the potential positive interactions with surface residues in the model created from pdb file 5e9d. The first alternative linker (GGAAGGSGGCPPCGGSGG) SEQ ID NO.19, was implemented for the Vα-linker-Vβ orientation. The change in the first alternative linker to alanine residues in position 3 and 4 of the linker (AA) was designed to interact with hydrophobic residues in the Vα domain, mainly in positions IMGT Vα 124 and IMGT Vα 125. These conserved hydrophobic residues form newly exposed surface hydrophobic patches after the constant TCR domains are removed in order to form an scTv. These mutations aim to stabilize the flexible scTv linker as well as reduce aggregation potential of these new hydrophobic sites.

The second alternative linker (GGGSDDSGGCPPCGGKGG), SEQ ID NO.18, was implemented in the Vβ-linker-Vα orientation. The hypothesis behind the second linker alteration was to generate stable interactions to minimize possible movement of the highly flexible unstructured linker and hence increase the stability of the scTV. Mutations in position 5 and 6 of the linker to aspartic acid (DD) were designed to form a salt bridge with a highly conserved arginine in position IMGT Vβ 123 among different TRBJ species. Furthermore, lysine (K) was proposed in position 16 of the second alternative linker to interact with semi-conserved glutamic acid in position IMGT Vα 3. The optimal linker connected distance between the first and second anchor points, measured by inter-alpha carbon distance for the anchor residues, lay in the range of 5 to 11.5 Angstroms (Table 1).

TABLE 1
TCE molecule scTv TCR chains, anchor positions, and CPPC-containing linkers, and modelled
anchor atomic distances.
Table 1A:

| | scTv | | Cys | | | | | | Cys | | Anchor |
|---|---|---|---|---|---|---|---|---|---|---|---|
| | SEQ | | Anchor 1 | | TRAC | | | | Anchor 2 | | 1-2 |
| Target | ID | | (IMGT | | frag- | | | | (IMGT | TRBC2 | (CA-CA) |
| PHLA | NO. | TRAV | p43) | TRAJ | ment | Linker | TRBV | TRBJ | p120) | frag | Angstrom |
| PRAME | 88 | TRAV12-2 | YSGCSPE | TRAJ48 | NIQN | GGGSGGSGG | TRBV5-1 | TRBJ2-7 | YFGCGTR | EDLKN | 5.5 |
| | | | SEQ ID | | SEQ ID | CPPCGGSGG | | | SEQ ID | SEQ ID | |
| | | | NO. 146 | | NO. 185 | SEQ ID | | | NO. 148 | NO. 189 | |
| | | | | | | NO. 17 | | | | | |
| PRAME | 89 | TRAV12-2 | YSGCSPE | TRAJ48 | NIQN | GGAAGGSGG | TRBV5-1 | TRBJ2-7 | YFGCGTR | EDLKN | 6.8 |
| | | | SEQ ID | | SEQ ID | CPPCGGSGG | | | | | |
| | | | NO. 146 | | NO. 185 | SEQ ID | | | SEQ ID | SEQ ID | |
| | | | | | | NO. 19 | | | | | |
| | | | | | | | | | NO. 148 | NO. 189 | |
| HIV- | 95 | TRAV12-2 | YSGCSPE | TRAJ41 | HIQN | EGGSGGSGG | TRBV5-6 | TRBJ2-7 | YFGCGTR | EDLKN | 6.8 |
| GAG | | | SEQ ID | | SEQ ID | CPPCGGSGG | | | SEQ ID | SEQ ID | |
| | | | NO. 146 | | NO. 186 | SEQ ID | | | NO. 148 | NO. 189 | |
| | | | | | | NO. 17 | | | | | |
| HIV- | 100 | TRAV12-2 | YSGCSPE | TRAJ | DIQN | GGGSGGSGG | TRBV6-5 | TRBJ2-7 | YFGCGTR | EDLIN | 6.5 |
| Tax | | | SEQ ID | 24-2 | SEQ ID | CPPCGGSGG | | | SEQ ID | SEQ 10 | |
| | | | NO. 146 | | NO. 187 | SEQ ID | | | NO. 148 | NO. 190 | |
| | | | | | | NO. 17 | | | | | |
| MAGE | 104 | TRAV5 | EPGCGLQ | TRAJ3 | NIQN | GGGSGGSGG | TRBV12-4 | TRBJ2-2 | FFGCGSR | EDLKN | 6.1 |
| | | | SEQ. ID | | SEQ ID | CPPCGGSGG | | | SEQ ID | SEQ ID | |
| | | | NO. 147 | | NO. 185 | SEQ ID | | | NO. 149 | NO. 189 | |
| | | | | | | NO. 17 | | | | | |

Table 1B:

| | scTv | | Cys | | | | | | Cys | | Anchor |
|---|---|---|---|---|---|---|---|---|---|---|---|
| | SEQ | | Anchor | | TRAC | | | | Anchor 2 | TRBC2 | 1-2 |
| Target | ID | | IMGT | | frag- | | | | (MGT | frag- | (CA-CA) |
| PHLA | NO. | TRAV | p47) | TRAJ | ment | Linker | TRBV | TRBJ | p120) | ment | Angstrom |
| PRAME | 136 | TRAV12-2 | YSCKSPE | TRAJ48 | NIQN | GGGSGGSGG | TRBV5-1 | TRBJ2-7 | YFGCGTR | EDLKN | 8.4 |
| | | | SEQ ID | | SEQ ID | CPPCGGSGG | | | SEQ ID | SEQ ID | |
| | | | NO. 150 | | NO. 185 | SEQ ID | | | NO. 152 | NO. 189 | |
| | | | | | | NO. 37 | | | | | |
| PRAME | 137 | TRAV12-2 | YSCKSPE | TRAJ48 | NIQN | GGAAGGSGG | TRBV5-1 | TRBJ2-7 | YFGCGTR | EDLKN | 9.5 |
| | | | SEQ ID | | SEQ ID | CPPCGGSGG | | | SEQ ID | SEQ ID | |
| | | | NO. 150 | | NO. 185 | SEQ ID | | | NO. 152 | NO. 189 | |
| | | | | | | NO. 19 | | | | | |
| HIV- | 138 | TRAV12-2 | YSCKSPE | TRAJ41 | HIQN | GGGSGGSGG | TRBV5-6 | TRBJ2-7 | YFGCGTR | EDLKN | 9.5 |
| GAG | | | SEQ ID | | SEQ ID | CPPCGGSGG | | | SEQ ID | SEQ ID | |
| | | | NO. 150 | | NO. 186 | SEQ ID | | | NO. 152 | NO. 189 | |
| | | | | | | NO. 37 | | | | | |
| HIV- | 139 | TRAV12-2 | YSCKSPE | TRAJ | DIQN | GGGSGGSGG | TRBV6-5 | TRBJ2-7 | YFGCGTR | EDLIN | 9.4 |
| Tax | | | SEQ ID | | SEQ ID | CPPCGGSGG | | | SEQ ID | SEQ 10 | |
| | | | NO. 150 | 24-2 | NO. 187 | SEQ ID | | | NO. 152 | NO. 190 | |
| | | | | | | NO. 17 | | | | | |
| MAGE | 140 | TRAV5 | EPCKGLQ | TRAJ3 | NIQN | GGGSGGSGG | TRBV12-4 | TRBJ2-2 | FFGCGSR | EDLKN | 8.9 |
| | | | SEQ ID | | SEQ ID | CPPCGGSGG | | | SEQ ID | SEQ ID | |
| | | | NO. 151 | | NO. 185 | SEQ ID | | | NO. 153 | NO. 189 | |
| | | | | | | NO. 17 | | | | | |

Table 1C:

| | scTv | | Cys | | | | | | Cys | | Anchor |
|---|---|---|---|---|---|---|---|---|---|---|---|
| | SEQ | | Anchor | | | | | | Anchor | | 1-2 |
| Target | ID | | IMGT | | TRBC2 | | | | 2 (MGT | TRAC | (CA-CA) |
| PHLA | NO. | TRBV | p47) | TRBJ | frag | Linker | TRAV | TRAJ | p120) | frag | Angstrom |
| PRAME | 85 | TRBV5-1 | QTPCQGL, | TRBJ2-7 | EDLKN | GGGSGGSGG | TRAV12-2 | TRAJ48 | TFGCGTR | NI | 10.1 |
| | | | SEQ ID | | SEQ ID | CPPCGGSGG | | | SEQ ID | SEQ ID | |
| | | | NO. 154 | | NO. 189 | SEQ ID | | | NO. 158 | NO: 182 | |
| | | | | | | NO. 17 | | | | | |
| PRAME | 86 | TRBV5-1 | QTPCQGL, | TRBJ2-7 | EDLKN | GGGSDDSGG | TRAV12-2 | TRAJ48 | TFGCGTR | NI | 7.9 |
| | | | SEQ ID | | SEQ ID | CPPCGGKGG | | | SEQ ID | SEQ ID | |
| | | | NO. 154 | | NO. 183 | SEQ ID | | | NO. 158 | NO: 182 | |
| | | | | | | NO. 18 | | | | | |
| HIV- | 93 | TRBV5-6 | QALCQGP, | TRBJ2-7 | EDLKN | GGGSGGSGG | TRAV12-2 | TRAJ41 | NFGCGTS | HI | 7.9 |
| GAG | | | SEQ ID | | SEQ ID | CPPCGGSGG | | | SEQ ID | SEQ ID | |
| | | | 80. 155 | | NO. 189 | SEQ ID | | | NO. 159 | NO: 183 | |
| | | | | | | NO. 17 | | | | | |
| HIV- | 98 | TRBV6-5 | QDPCMGL, | TRBJ2-7 | EDLIN | GGGSGGSGG | TRAV12-2 | TRAJ | QFGCGTQ | DI | 6.5 |
| Tax | | | SEQ ID | | SEQ ID | CPPCGGSGG | | 24-2 | SEQ ID | SEQ ID | |
| | | | NO. 156 | | NO. 190 | SEQ ID | | | NO. 160 | NO: 184 | |
| | | | | | | NO. 17 | | | | | |
| MAGE | 102 | TRBV12-4 | QTMCRGL, | TRBJ2-4 | EDLKN | GGGSGGSGG | TRAV5 | TRAJ3 | IFGCGTR | NI | 9.7 |
| | | | SEQ ID | | SEQ ID | CPPCGGSGG | | | SEQ ID | SEQ ID | |
| | | | NO. 157 | | NO. 189 | SEQ ID | | | NO. 161 | NO: 182 | |
| | | | | | | NO. 17 | | | | | |

Table 1D:

| | scTv | | Cys | | | | | | Cys | | Anchor |
|---|---|---|---|---|---|---|---|---|---|---|---|
| | SEQ | | Anchor | | | | | | Anchor | | 1-2 |
| Target | ID | | IMGT | | TRBC2 | | | | 2 (MGT | TRAC | (CA-CA) |
| PHLA | NO. | TRBV | p46) | TRBJ | frag | Linker | TRAV | TRAJ | p120) | frag | Angstrom |
| PRAME | 141 | TRBV5-1 | QTCGQGL | TRBJ2-7 | EDLKN | GGGSGGSGG | TRAV12-2 | TRAJ48 | TFGCGTR | NI | 10.4 |
| | | | SEQ ID | | SEQ ID | CPPCGGSGG | | | SEQ ID | SEQ ID | |
| | | | NO. 162 | | NO. 189 | SEQ ID | | | NO. 158 | NO: 182 | |
| | | | | | | NO. 17 | | | | | |
| PRAME | 142 | TRBV5-1 | QACGQGP | TRBJ2-7 | EDLKN | GGGSDDSGG | TRAV12-2 | TRAJ48 | TFGCGTR | NI | |
| | | | SEQ ID | | SEQ ID | CPPCGGKGG | | | SEQ ID | SEQ ID | 10.8 |
| | | | NO. 163 | | NO. 183 | SEQ ID | | | NO. 158 | NO: 182 | |
| | | | | | | NO. 18 | | | | | |
| HIV- | 143 | TRBV5-6 | QDCGMGL | TRBJ2-7 | EDLKN | GGGSGGSGG | TRAV12-2 | TRAJ41 | NFGCGTS | HI | |
| GAG | | | SEQ ID | | SEQ ID | CPPCGGSGG | | | SEQ ID | SEQ ID | 10.8 |
| | | | NO. 164 | | NO. 189 | SEQ ID | | | NO. 159 | NO: 183 | |
| | | | | | | NO. 17 | | | | | |
| HIV- | 144 | TRBV6-5 | QTCMRGL | TRBJ2-7 | EDLIN | GGGSGGSGG | TRAV12-2 | TRAJ | QFGCGTQ | DI | |
| Tax | | | SEQ ID | | SEQ ID | CPPCGGSGG | | 24-2 | SEQ ID | SEQ ID | 9.1 |
| | | | NO. 165 | | NO. 190 | SEQ ID | | | NO. 160 | NO: 184 | |
| | | | | | | NO. 17 | | | | | |
| MAGE | 145 | TRBV12-4 | QTCMRGL | TRBJ2-4 | EDLKN | GGGSGGSGG | TRAV5 | TRAJ3 | IFGCGTR | NI | |
| | | | SEQ ID | | SEQ ID | CPPCGGSGG | | | SEQ ID | SEQ ID | 11.2 |
| | | | NO. 166 | | NO. 189 | SEQ ID | | | NO. 161 | NO: 182 | |
| | | | | | | NO. 17 | | | | | |

### Example 2: Bispecific Polypeptide Production

All TCE molecules included in the embodiments of the present invention contain two chains held together by KiH Fc molecules (see FIG. 1). Plasmid DNAs encoding these chains with appropriate signal peptides were created by using standard methods, and multiple mammalian cell line transfection systems were used to make these molecules from these plasmids according to one of three methodologies set out in Methods 2α to 2c below.
Methods: 2a) The TCE two-chain bispecific polypeptides were expressed in HEK293 cells by transient gene expression. Suspension culture-adapted HEK293 cells were co-transfected with the bispecific expression vector encoding plasmids and cultured for 6 days on a fed-batch production run. Expression of intact chain paired molecules was confirmed 72 hours after transfection using SDS PAGE on the cell culture crude supernatants. Supernatants were harvested and filtrated before affinity chromatography (Protein A) followed by cation exchange (CIEX) and/or size exclusion chromatography (SEC) using superdex 200™ Increase 5/150 GL. Purified proteins were stored in either PBS or 20 mM Histidine pH6.0. All proteins were filtered through 0.22 uM filters for sterilization prior to aliquoting and freezing prior to use in cell culture experiments. These molecules were used for the exampled cytotoxicity analysis.
Methods: 2b) Alternatively, the two-chain bispecific polypeptides were expressed in suspension-adapted CHO cells by transient gene expression. CHO cells were co-transfected with the bispecific expression vector encoding plasmids and cultured for 6 days on a fed-batch production run. Supernatants were harvested and filtered prior to affinity chromatography using MabSelect-SURE™ alkali-tolerant protein A. Followed by HPLC-SEC. Purified proteins were stored in PBS with 100 mM L-Arginine at pH 6.5-6.7 and filtered through 0.22 uM filters for sterilisation prior to aliquoting and freezing prior to use in SPR and stability studies. These molecules were used for the exampled thermal stability and SPR analysis.
Methods: 2c) The two-chain bispecific polypeptide encoding sequences were chemically synthetised and subcloned into separate mammalian expression plasmids (containing CMV enhancer/promoter, a mammalian signal peptide, and a chimeric intron). ExpiCHO-S™ cells were co-transfected with the two plasmids (one for each chain) using the ExpiCHO™ system (Thermo) and 8-10 days repetitive fed-batch expression runs were done at 48-mL scale on shaking Erlenmeyer flasks. Expression of intact chain paired molecules was confirmed 72-96 hours after transfection using SDS PAGE (gradient gel 4-10%) on the cell culture crude supernatants. Supernatants were harvested by centrifugation and filtrated using 0.22 um vacuum filters. Then, a first two consecutive affinity chromatography steps were done using (A) Hi-Trap™ Protein A 5-mL column (Cytiva™) and (B) HiTrap™ MabSelect™ VL 5-mL column (to enrich purity and remove potential by-products). Finally, TCE molecules were concentrated using VivaSpin™ columns (Sartorius™) and polished using a 320 mL (26/600) 200pg SEC column (Cytiva™). Purified proteins were suspended in PBS at final concentration of 2-10 uM, purity was confirmed using SDS-PAGE/SEC-HPLC (>97.5% pure) and Endotoxin content was measured confirming values of <0.5 EU/mg. Final samples were aliquoted and stored at −80° C. These molecules were used for the exampled IFNg production, thermal stability and SPR analysis.

### Example 3: Biophysical Characterization of the TCE Bispecific Engager Molecules

Multiple methods were used to characterize the properties of the mammalian cell expressed TCE molecules.
Methods 3a) Thermal stability of TCE molecules: The thermal stability of the purified TCE molecules was assessed using a Prometheus Panta™ nano differential scanning fluorimetry instrument. The unfolding temperatures, which may be also referred to as melting temperatures, transition temperatures, or inflection points, were assigned by analysing the first derivative of the ratio of fluorescence emission intensities at 350 and 330 nm. The summits of the peaks in the first-derivative graph were assigned as the unfolding temperatures of different domains of the samples according to prior knowledge and literature values. These temperatures represent the temperatures at which the protein domain unfolds at the highest rate. The experiments were carried out using the following parameters: 1) temperature range: 25-95° C. 2) ramping rate: 1° C./min.
Methods 3b) Binding of TCE molecules to pHLA: The binding kinetics of the TCR domains of the TCE molecules s were measured using a Cytiva Biacore 3000™ surface plasmon resonance instrument. The samples were immobilised on the Protein A sensor chip by their human Fc domains. The densities of the immobilised samples were optimised to avoid mass transfer limitation while keeping the binding signal level in the range suitable for the instrument. The sensor chip was kept at 37° C. The kinetics was measured by 5 successive injections of the peptide-human leukocyte antigen (pHLA) complex with increasing concentrations in an experimental design typically known as single-cycle kinetics. The concentration ranges of the pHLAs were designed so that the highest concentration is about 10-fold of the dissociation constant (KD), and the lowest concentration is sufficiently lower than the KD. In some instances, a wide range of concentrations were trialled first to estimate the KD and then a narrower range was used to accurately measure the KD. The duration of the association and dissociation phases were optimised so that the highest pHLA concentration reaches the signal plateau and at least 10% of bound pHLA was dissociated, respectively. The data were fitted to a 1:1 Langmuir binding model. The model fitting was inspected visually. In addition, the chi-squared value must be below 10% of the R-max value for the model fitting to be considered acceptable. KD was calculated by dividing the dissociate rate (k-off, measured in the unit s⁻¹) by the associate rate (k-on, measured in the unit M⁻¹s⁻¹).

### Results 3a) TCE Molecule Thermal Stability

The thermal stability of various TCE molecules (and derivatives thereof; not shown) was established using standard methods, these methodologies indicated that the first thermal transition temperature of TCE molecules was due to the unfolding of the scTv. As shown in Table 2, introduction of various CPPC-containing linkers (see Table 1) into PRAME-specific TCE molecules containing both Va-Vb and Vb-Va orientations produced molecules with increased thermal stability, as shown by an increase in the temperature of the first thermal transition. Interestingly, the first thermal transition displayed by the CPPC-linked TCE molecules exceeded that of a TCER molecule created from the same Va and Vb chains (and equivalent VL and VH chains) (Table 2). Importantly, this thermal stability increase was also observed in an increase in the first thermal transition of a CPPC-linked version of an HIV-GAG-specific TCE molecule, relative to the G4S4-linked version (Table 2). CPPC-linked and G4S4-linked versions of an HIV-TAX-specific (N022 series TCE molecules, SEQ ID NO.29 & SEQ ID NO.96-100) and MAGE-A4-specific (N022 series TCE molecules, SEQ ID NO.29 & SEQ ID NO.101-104) TCE molecules were also produced and purified for target binding and thermal stability assay. In conclusion the introduction of the scTv CPPC based linker enhances the resulting stability of the TCE molecule, the effect is consistent across different scTvs as demonstrated for both PRAME and HIV epitope targeting scTvs. Stability is enhanced over that of the comparator TCER molecule.

Results 3b) Binding of TCE to pHLA:

The binding of various TCE molecules to their pHLA targets was established using standard methods. As shown in Table 2, introduction of various CPPC-containing linkers (see Table 1) into PRAME-specific TCE molecules containing both Va-Vb and Vb-Va orientations produced molecules with increased affinity for their pHLA target relative to molecules with a G4S4 linker. Interestingly, the affinity achieved in these assays for CPPC-linked versions of these TCE molecules was equal to the affinity measured for the comparator TCER molecule created from the same Va and Vb chains. In this assay, the pHLA binding affinity of a CPPC-linked version of a very high affinity HIV-GAG-specific TCE molecule was not significantly enhanced by addition of the CPPC linker, but the CPPC linker did not have a detrimental effect on its binding affinity for its pHLA ligand (Table 2). In conclusion the presence of the CPPC linker within the scTv element of the TCE bispecific molecule has demonstrated a measurable enhancement to the binding affinity to the pMHC target, the effect of this enhancement being more pronounced at lower starting Kd (i.e. in absence of CPPC linker). The measured endpoint is equivalent for both the comparator TCER molecules and the TCE with CPPC linker.

TABLE 2
Influence of linker stapling on thermal stability
and binding to target pHLA:

| | Chain | Chain | | Tran- | |
|---|---|---|---|---|---|
| | 1 | 2 | | sition | |
| | SEQ | SEQ | | temp 1 | pHLA |
| Molecule | ID | ID | | (scTv; | KD |
| ID | NO. | NO. | Molecule Description | C.) | (nM) |
| N010_009 | 29 | 30 | PRAME TCE scFv-KiH- | 50.3 | 15.7 |
| | | | scTv (Vb-G4S4-Va) | | |
| N010_010 | 29 | 85 | PRAME TCE scFv-KiH- | 57.0 | 8.1 |
| | | | scTv (Vb-CPPC-Va) | | |
| N010_011 | 29 | 86 | PRAME TCE scFv-KiH- | 58.7 | 11.7 |
| | | | scTv (Vb-DDK-CPPC-Va) | | |
| N010_018 | 29 | 87 | PRAME TCE scFv-KiH- | 51.2 | 15.6 |
| | | | scTv (Va-G4S4-Vb) | | |
| N010_014 | 29 | 88 | PRAME TCE scFv-KiH- | 55.4 | 5.3 |
| | | | scTv (Va-CPPC-Vb) | | |
| N010_015 | 29 | 89 | PRAME TCE scFv-KiH- | 55.2 | 7.6 |
| | | | scTv (Va-AA-CPPC-Vb) | | |
| N011_003 | 35 | 36 | PRAME TCER | 53.9 | 7.7 |
| N021_009 | 29 | 92 | HIV-GAG TCE scFv-KiH- | 54.5 | 1.2 |
| | | | scTv (Vb-G4S4-Va) | | |
| N021_010 | 29 | 93 | HIV-GAG TCE scFv-KiH- | 63.9 | 1.2 |
| | | | scTv (Vb-CPPC-Va) | | |

### Example 4: Bispecific Polypeptide Activity Assays

To evaluate the functionality of the TCR based bi-specific molecules (TCEs), co-culture assays were set up to measure IFNy release and target cytotoxicity. Each bi-specific molecule contained the same scTv recognising the peptide HLA complex HLA-A2-PRAME peptide SLLQHLIGL (SEQ ID NO.81). Target cells used were PRAME positive MEL624 cells and PRAME negative 92-1 cells with PBMCS used as effector cells. Assays were carried out in RPMI containing 10% FBS. The bispecific molecule TCER comprising polypeptide chain sequences SEQ ID No.35 and 36 and as disclosed in WO2021023657 was used as a comparator.

### (a) IFNy Release Assay

For the IFNy release assay, 50,000 T2 cells were plated in flat-bottomed 96 well plates and allowed to adhere for 2 hours followed by addition of 5 nM peptide, final concentration. PBMCS were thawed and rested for 2 hours. 50,000 PBMC plus bi-specific molecules ranging from 10fM to 100 nM were added to plates containing target cells. Co-cultures were incubated at 37° C. in 5% CO₂ for 48 hours, before supernatant was harvested and analysed for IFNy using Human IFNγ DuoSet™ ELISA (R&D Systems™). All ELISAs were performed as per manufacturer's instructions.

### (b) Cytotoxicity Assay

Cytotoxicity was measured using the Incucyte SX5™ and Incucyte™ Caspase—3/7 Green Dye for Apoptosis (Sartorius™). 20,000 target cells were plated in flat-bottomed 96 well plates and allowed to adhere overnight. PBMCS were thawed and rested for 2 hours. 100,000 PBMC plus bi-specific molecules ranging from 1 μM to 10 nM were added to plates containing target cells. Assay media contained 5 μM Caspase—3/7 Green Dye for Apoptosis. Co-cultures were incubated at 37° C. in 5% CO₂ inside the Incucyte SX5 for 72 hours and images were taken every 3 hours. Incucyte 2021 C software was used to analyse images and generate counts of number of apoptotic target cells per image.

### Results:

### (a) Hinge Length Testing:

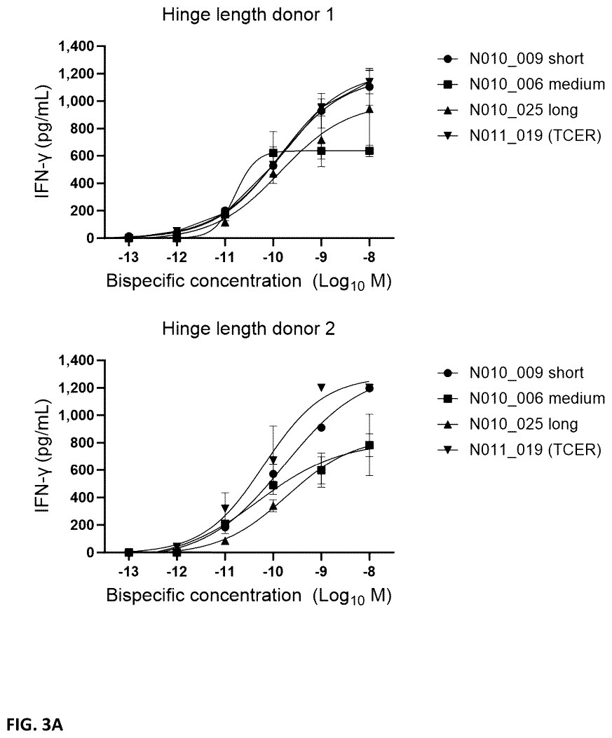

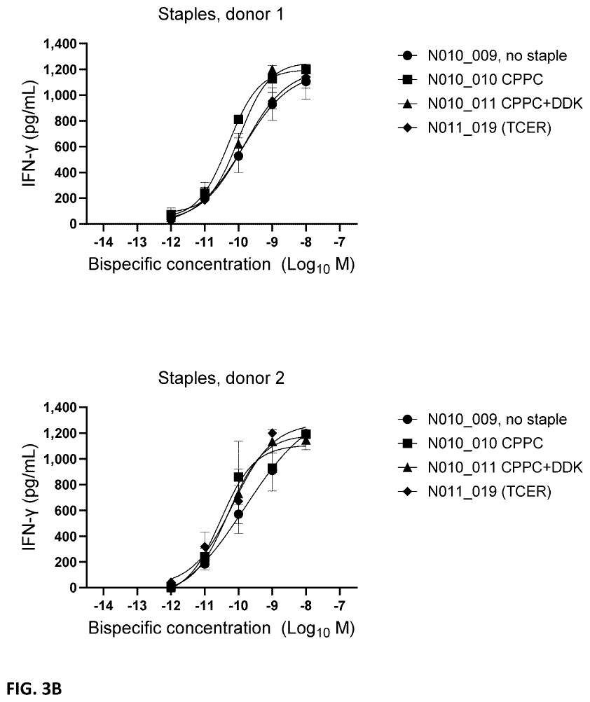

The effect of varying the hinge length of TCE designs provided by molecules N010_009 (SEQ ID NO.29 & SEQ ID NO.30) short hinge, N010_006 (SEQ ID NO.105 & SEQ ID NO.84) medium hinge and N010_025 (SEQ ID NO.106 & SEQ ID NO.91) long hinge [see FIG. 1], was investigated using an IFNy release assay. T2 were incubated with PRAME peptide, PBMCs plus bi-specific molecules ranging from 10fM to 100 nM. IFNg was measured at 48 hours. The data (FIG. 3A) show that incorporation of a short hinge, as exemplified with design N010_009 gives the best efficacy in terms of IFNg response of PBMC in response to PRAME peptide presented by T2 cells. The extent of IFNg release is very close to that of the comparator TCER molecule N011_019 (SEQ ID NO.134 & SEQ ID NO.135) across 5 log titration of the TCE.

### (b) CPPC Linker Stabilization:

The functional value of stabilising the scTv of the TCE was investigated by varying this domain of N010_009 (SEQ ID NO.29 & SEQ ID NO.30) by incorporation of a CPPC (molecule N010_010, SEQ ID NO.29 & SEQ ID NO.85) or CPPC+DDK staple (molecule N010_011, SEQ ID NO.29 & SEQ ID NO.86). Using the T2 cellular assay described above, the IFNg data (FIG. 3B) showed that incorporation of a CPPC or a CPPC+DDK staple enhanced the functional activity of the TCE to a small but reproducible degree.

### (c) N010_009 Versus TCER Comparator

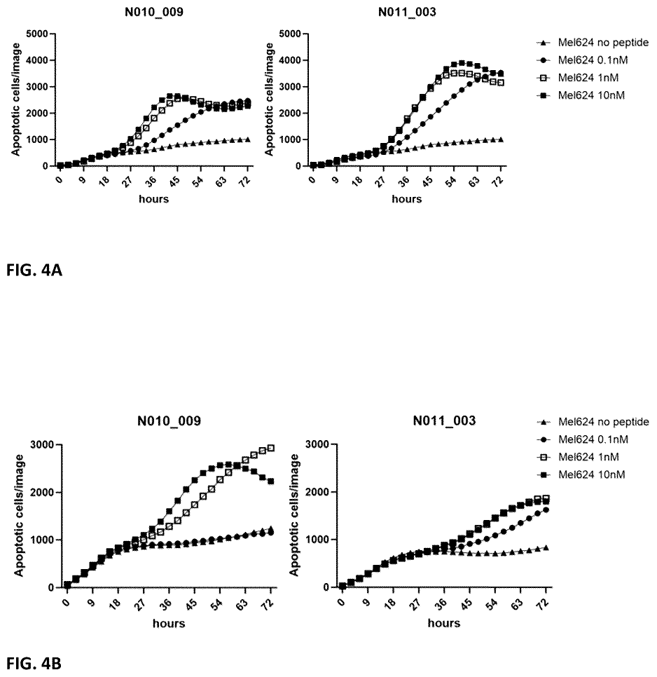

The relative efficacy of redirected killing of a PRAME positive cell target by the N010_009 (SEQ ID NO.29 & SEQ ID NO.30) TCE format was compared to the TCER N011_003 format (SEQ ID NO.35 & SEQ ID NO.36). Using the Incucyte™ assay and titrating the TCEs the timing of induction of killing, the degree of titration and degree of killing response is similar between the N010_009 format and N011_003 comparator, across two donors (FIGS. 4A and 4B).

### Conclusions:

The bispecific molecule N010_009 (SEQ ID NO.29 & SEQ ID NO.30) demonstrates specificity for cell targets expressing the PRAME antigen (SEQ ID NO.81) and an ability to engage an immune effector cell response as measured by cytokine release assay. N010_009 demonstrates the most similar activity to the comparator PRAME TCER molecule N011_003 in terms of time to peak kill, (FIGS. 4 and 5) this is most clearly seen in the Donor 1 data. The results confirm that the TCE molecule N010_009 offers an excellent basis as a therapeutic bispecific for cancer therapy.

Investigations into the role of hinge length in the N010_009 series employed three molecules with identical structures except for differences in hinge length, [molecules N010_009 (SEQ ID NO.29 & SEQ ID NO.30) short hinge, N010_006 (SEQ ID NO.105 & SEQ ID NO.84) medium hinge and N010_025 (SEQ ID NO.106 & SEQ ID NO.91) long hinge, see FIG. 1]. These studies documented that the length of the hinge was a key factor in this particular scFv-KiH-scTv format. The best efficacy in functional response assays was demonstrated with the short hinge which would produce the shortest synapse distance between an antigen presenting cell (APC) or tumour cell and a T-cell, the functional results of this assay being very similar to those of the comparator TCER molecule N011_019 (SEQ ID NO.134 & SEQ ID NO.135). The short hinge has a distance between the ends of the scTv and scFv of around 2.1 nm (comprising 4 peptide bonds and one cys=cys disulphide bridge) in comparison to the medium hinge of around 5.9 nm (comprising 14 peptide bonds and one cys=cys disulphide bridge) and the long hinge of around 9.7 nm (comprising 24 peptide bonds and one disulphide bridge). The distance discussed is the region spanning or connecting the terminal amino acids of the variable domains of the scTv and the scFv. The data points to the optimal distance as being in the range greater than or equal to 2.1 nm and less than 5.9 nm or greater than or equal to (cys=cys & 4 peptide bonds) but less than (cys-cys & 14 peptide bonds). Improvement in functional response was further demonstrated with the short hinge TCE N010_009 by introduction of a stabilising linker sequence into the scTv such that the Va and Vb domains are flexibly crosslinked, i.e. by incorporation of a CPPC staple (molecule N010_010, SEQ ID NO.29 & SEQ ID NO.85) or CPPC+DDK staple (molecule N010_011, SEQ ID NO.29 & SEQ ID NO.86).

In conclusion, the present data has shown that this TCE molecule provides excellent recognition and functional effectiveness promoting immune response to a number of diverse peptide epitope presented by MHC derived from different tumour and cancer types, a selection exampled here by MAGE-A4, HIV-GAG, HIV-TAX and PRAME epitopes. The functional data analysis presented here supports the proposal that the short hinge TCE N010_009 of the invention, optionally comprising the scTv linker CPPC or CPPC+DDK, offers an excellent basis as a therapeutic bispecific for cancer therapy which may be directed to a wide variety of MHC presented antigens in the context of treating a diverse set of tumours and cancers.

SEQUENCES
SEQ ID NO. 1
DIQMTQSPSSLSASVGDRVTITCRASQDIRNYLNWYQQKPGKAPKLLIYYTSRLESGVPSRFSGSGSGTDYTLTISSLQ
PEDFATYYCQQGNTLPWTFGQGTKVEIK.
(UCHT1.v9. VL)
CDRs underlined.
SEQ ID NO. 2
EVQLVESGGGLVQPGGSLRLSCAASGYSFTGYTMNWVRQAPGKGLEWVALINPYKGVSTYNQKFKDRFTISVDKSK
NTAYLQMNSLRAEDTAVYYCARSGYYGDSDWYFDVWGQGTLVTVSS.
(UCHT1.v9. VH)
CDRs underlined.
RASQDIRNYLN,
SEQ ID NO. 3
(UCHT1 VL CDR1),
SEQ ID NO. 4
YYTSRLES,
(UCHT1 VL CDR2),
SEQ ID NO. 5
QQGNTLPWT
(UCHT1 VL CDR3)
SEQ ID NO. 6
GYTMN,
(UCHT1 VH CDR1),
SEQ ID NO. 7
LINPYKGVSTYNQKFKD,
(UCHT1 VH CDR2),
SEQ ID NO. 8
SGYYGDSDWYFDV,
(UCHT1 VH CDR3)
SEQ ID NO. 9
QKEVEQNSGPLSVPEGAIASLNCTYSDRGSQSFFWYRQYSGKSPELIMSIYSNGDKEDGRFTAQLNKASQYVSLLIRD
SQPSDSATYLCAAVIDNDQGGILTFGTGTRLTIIPNI,
(Tv alpha (PRAME), [Vα]). CDRs
underlined. Q at position 1 may be replaced by G in some embodiments.
SEQ ID NO. 10
KAGVTQTPRYLIKTRGQQVTLSCSPIPGHRAVSWYQQTPGQGLQFLFEYVHGEERNKGNFPGRFSGRQFSNSSSE
MNISNLELGDSALYLCASSPWDSPNVQYFGPGTRLTVTEDLKN,
(Tv beta (PRAME), [Vβ]).
CDRs underlined
SEQ ID NO. 11
DRGSQS,
(Tv alpha, CDR1).
SEQ ID NO. 12
IYSNGD,
(Tv alpha, CDR2).
SEQ ID NO. 13
AAVIDNDQGGILT,
(Tv alpha, CDR3).
SEQ ID NO. 14
PGHRA,
(Tv beta, CDR1).
SEQ ID NO. 15
YVHGEE,
(Tv beta, CDR2).
SEQ ID NO. 16
ASSPWDSPNVQY,
(Tv beta, CDR3).
SEQ ID NO. 17
GGGSGGSGGCPPCGGSGG,
linker.
SEQ ID NO. 18
GGGSDDSGGCPPCGGKGG,
linker.
SEQ ID NO. 19
GGAAGGSGGCPPCGGSGG,
linker.
SEQ ID NO. 20
EPKSCDKTHTCPPCPAPELLG,
IgG1 hinge.
SEQ ID NO. 21
ERKCCVECPPCPAPPVAGP,
IgG2 hinge.
SEQ ID NO. 22
ESKYGPPCPSCPAPEFLG,
IgG4 hinge.
SEQ ID NO. 23
CPPCPAPELLG,
IgG1 hinge fragment.
SEQ ID NO. 24
CPPCPAPEAAG,
IgG1 hinge fragment variant.
SEQ ID NO. 25
CPPCPAPEAAGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALGAPIEKTISKAKGQPREPQVYTLPPCRDELTKNQVSLWCLVKGFYPSDIAV
EWESNGQPENNYKTTPPVLDSDGSFFLYSKLTVDKSRWQQGNVFSCSVMHEALHNHYTQKSLSLSP,
(Fc Knob, including hinge, truncated with C-terminal GK deletion); hinge underlined.
SEQ ID NO. 26
PSVFLFPPKPKDTLMISRTPEVTCVVVDVSHEDPEVKFNWYVDGVEVHNAKTKPREEQYNSTYRVVSVLTVLHQD
WLNGKEYKCKVSNKALGAPIEKTISKAKGQPREPQVYTLPPCRDELTKNQVSLWCLVKGFYPSDIAVEWESNGQPE
NNYKTTPPVLDSDGSFFLYSKLTVDKSRWQQGNVFSCSVMHEALHNHYTQKSLSLSP,
(Fc Knob, excluding hinge, truncated with C-terminal GK deletion).
SEQ ID NO. 27
CPPCPAPEAAGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALGAPIEKTISKAKGQPREPQVCTLPPSRDELTKNQVSLSCAVKGFYPSDIAV
EWESNGQPENNYKTTPPVLDSDGSFFLVSKLTVDKSRWQQGNVFSCSVMHEALHNHYTQKSLSLSP,
(Fc Hole, including hinge, truncated with C-terminal GK deletion); hinge underlined.
SEQ ID NO. 28
PSVFLFPPKPKDTLMISRTPEVTCVVVDVSHEDPEVKFNWYVDGVEVHNAKTKPREEQYNSTYRVVSVLTVLHQD
WLNGKEYKCKVSNKALGAPIEKTISKAKGQPREPQVCTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSLSP,
(Fc Knob, excluding hinge, truncated with C-terminal GK deletion); or
SEQuence variant including the amino
acid residues GK at the C-terminus.
SEQ ID NO. 29
DIQMTQSPSSLSASVGDRVTITCRASQDIRNYLNWYQQKPGKAPKLLIYYTSRLESGVPSRFSGSGSGTDYTLTISSLQ
PEDFATYYCQQGNTLPWTFGQGTKVEIKGGGGSGGGGSGGGGSGGGGSEVQLVESGGGLVQPGGSLRLSCAASG
YSFTGYTMNWVRQAPGKGLEWVALINPYKGVSTYNQKFKDRFTISVDKSKNTAYLQMNSLRAEDTAVYYCARSGYY
GDSDWYFDVWGQGTLVTVSSCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALGAPIEKTISKAKGQPREPQVYTLPPCRDE
LTKNQVSLWCLVKGFYPSDIAVEWESNGQPENNYKTTPPVLDSDGSFFLYSKLTVDKSRWQQGNVFSCSVMHEAL
HNHYTQKSLSLSP,
scFv arm [UCHT1 VL-(G₄S)₄-UCHT1-VH-Fc Knob truncated with C-
terminal GK deletion]. G4S4 linker underlined italic, hinge underlined.
SEQ ID NO. 30
KAGVTQTPRYLIKTRGQQVTLSCSPIPGHRAVSWYQQTPGQGLQFLFEYVHGEERNKGNFPGRFSGRQFSNSSSE
MNISNLELGDSALYLCASSPWDSPNVQYFGPGTRLTVTEDLKNGGGGSGGGGSGGGGSGGGGSQKEVEQNSGPL
SVPEGAIASLNCTYSDRGSQSFFWYRQYSGKSPELIMSIYSNGDKEDGRFTAQLNKASQYVSLLIRDSQPSDSATYLC
AAVIDNDQGGILTFGTGTRLTIIPNICPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE
ALHNHYTQKSLSLSP,
scTv arm [Vb-(G₄S)₄-Va-Fc Hole truncated with C-terminal GK
deletion]. G4S4 linker underlined italic, hinge underlined.
SEQ ID NO. 31
KAGVTQTPRYLIKTRGQQVTLSCSPIPGHRAVSWYQQTPGQGLQFLFEYVHGEERNKGNFPGRFSGRQFSNSSSE
MNISNLELGDSALYLCASSPWDSPNVQYFGPGTRLTVTEDLKNGSADDAKKDAAKKDGKSQKEVEQNSGPLSVPE
GAIASLNCTYSDRGSQSFFWYRQYSGKSPELIMSIYSNGDKEDGRFTAQLNKASQYVSLLIRDSQPSDSATYLCAAVI
DNDQGGILTFGTGTRLTIIPNICPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALGAPIEKTISKAKGQPREPQVCTLPPSRDEL
TKNQVSLSCAVKGFYPSDIAVEWESNGQPENNYKTTPPVLDSDGSFFLVSKLTVDKSRWQQGNVFSCSVMHEALH
NHYTQKSLSLSP,
scTv arm [Vb-(Kranz Linker)-Va-Fc Hole truncated with C-terminal
GK deletion]. Kranz linker underlined italic, hinge underlined.
SEQ ID NO. 32
APELLGGPSVFLEPPKPKDTLMISRTPEVTCVVVDVSHEDPEVKFNWYVDGVEVHNAKTKPREEQYNSTYRVVSVLT
VLHQDWLNGKEYKCKVSNKALGAPIEKTISKAK,
[CH2 region residues 231-340 IgG1, EU numbering].
SEQ ID NO. 33
APPVA-GPSVFLFPPKPKDTLMISRTPEVTCWVDVSHEDPEVKFNWYVDGVE
VHNAKTKPREEQYQSTYRVVSVLTVLHQDWLNGKEYKCKVSNKALPASIEK-,
[CH2 region
variant residues 231-340 IgG1, EU numbering].
SEQ ID NO. 34
APPVA-GPSVFLFPPKPKDTLMISRTPEVTCWVDVSHEDPEVKFNWYVDGVE
VHNAKTKPREEQYNSTYRWSVLTVLHQDWLNGKEYKCKVSNKALPASIEK-,
[CH2 region
variant residues 231-340 IgG1, EU numbering]
SEQ ID NO. 35
EVQLVQSGAEVKKPGASVKVSCKASGYSFTGYTMNWVRQAPGQGLEWMGLINPYKGVSTYAQKFQDRVTLT
VDKSTSTAYMELSSLRSEDTAVYYCARSGYYGDSDWYFDVWGQGTLVTVSSGGGSGGGGKAGVTQTPRYLI
KTRGQQVTLSCSPIPGHRAVSWYQQTPGQGLQFLFEYVHGEERNKGNFPGRFSGRQFSNSSSEMNISNLEL
GDSALYLCASSPWDSPNVQYFGPGTRLTVTEDLKNEPKSSDKTHTCPPCPAPPVAGPSVFLFPPKPKDTLM
ISRTPEVTCVVVDVSHEDPEVKFNWYVDGVEVHNAKTKPREEQYQSTYRVVSVLTVLHQDWLNGKEYKCKV
SNKALPASIEKTISKAKGQPREPQVCTLPPSRDELTKNQVSLSCAVKGFYPSDIAVEWESNGQPENNYKTT
PPVLDSDGSFFLVSKLTVDKSRWQQGNVFSCSVMHEALHNHYTQKSLSLSPGK,
PRAME TCER
Chain 1 [UCHT1 VH-Linker-Vbeta (prame)-Fc].
Linker underlined, Fc underlined italic.
SEQ ID NO. 36
QKEVEQNSGPLSVPEGAIASLNCTYSDRGSQSFFWYRQYSGKSPELIMSIYSNGDKEDGRFTAQLNKASQY
VSLLIRDSQPSDSATYLCAAVIDNDQGGILTFGTGTRLTIIPNIQNGGGSGGGGDIQMTQSPSSLSASVGD
RVTITCRASQDIRNYLNWYQQKPGKAPKLLIYYTSRLHSGVPSRFSGSGSGTDYTLTISSLQPEDIATYFC
QQGQTLPWTFGQGTKVEIKEPKSSDKTHTCPPCPAPPVAGPSVFLFPPKPKDTLMISRTPEVTCVVVDVSH
EDPEVKFNWYVDGVEVHNAKTKPREEQYQSTYRVVSVLTVLHQDWLNGKEYKCKVSNKALPASIEKTISKA
KGQPREPQVYTLPPCRDELTKNQVSLWCLVKGFYPSDIAVEWESNGQPENNYKTTPPVLDSDGSFFLYSKL
TVDKSRWQQGNVFSCSVMHEALHNHYTQKSLSLSPGK,
PRAME TCER Chain 2, [Valpha
(prame)-Linker-UCHT1 VL-Fc]. Linker underlined, Fc underlined italic.
SEQ ID NO. 37
GSAGSAAGSGEF,
linker.
SEQ ID NO. 38
KESGSVSSEQLAQFRSLD,
linker.
SEQ ID NO. 39
EGKSSGSGSESKST,
linker.
SEQ ID NO. 40
GGGSGGGG,
linker.
SEQ ID NO. 41
GGGS,
linker.
GGGGS,
SEQ ID NO. 42
linker.
SEQ ID NO. 43
TVLRT,
linker.
SEQ ID NO. 44
TVSSAS,
linker.
SEQ ID NO. 45
TVLSSAS,
linker.
SEQ ID NO. 46
GGGGSGGGGSGGGGSGGGGS,
G4S4 linker
SEQ ID NO. 47
GSADDAKKDAAKKDGKS,
Kranz linker
(SEQ ID NO: 48)
C(X)yC,
LNK1 type sequence.
(SEQ ID NO: 50)
CGC,
LNK1 type sequence.
(SEQ ID NO: 51)
CSC,
LNK1 type sequence.
(SEQ ID NO: 52)
CPPC,
LNK1 type sequence.
(SEQ ID NO: 53)
CGPC,
LNK1 type sequence.
(SEQ ID NO: 54)
CPGC,
LNK1 type sequence.
(SEQ ID NO: 55)
CGGC,
LNK1 type sequence.
(SEQ ID NO: 56)
CSPG,
LNK1 type sequence.
(SEQ ID NO: 57)
CPSC,
LNK1 type sequence.
(SEQ ID NO: 58)
CSSC,
LNK1 type sequence.
(SEQ ID NO: 59)
CGSC,
LNK1 type sequence.
(SEQ ID NO: 60)
CSGC,
LNK1 type sequence.
(SEQ ID NO: 61)
CPPPC,
LNK1 type sequence.
(SEQ ID NO: 62)
CGPPC,
LNK1 type sequence.
(SEQ ID NO: 63)
CPGPC,
LNK1 type sequence.
(SEQ ID NO: 64)
CPPGC,
LNK1 type sequence.
(SEQ ID NO: 65)
CGGPC,
LNK1 type sequence.
(SEQ ID NO: 66)
CPGGC,
LNK1 type sequence.
(SEQ ID NO: 67)
CGGGC,
LNK1 type sequence.
(SEQ ID NO: 68)
CSPPC,
LNK1 type sequence.
(SEQ ID NO: 69)
CPSPC,
LNK1 type sequence.
(SEQ ID NO: 70)
CPPSC,
LNK1 type sequence.
(SEQ ID NO: 71)
CSSPC,
LNK1 type sequence.
(SEQ ID NO: 72)
CPSSC,
LNK1 type sequence.
(SEQ ID NO: 73)
CSSSC,
LNK1 type sequence.
(SEQ ID NO: 74)
CGSPC,
LNK1 type sequence.
(SEQ ID NO: 75)
CPGSC,
LNK1 type sequence.
(SEQ ID NO: 76)
CSGPC,
LNK1 type sequence.
(SEQ ID NO: 77)
CPSGC,
LNK1 type sequence.
(SEQ ID NO. 78)
CPPCPAPEAAGG,
SHORT HINGE.
(SEQ ID NO. 79)
DKTHTCPPCPAPEAAGG,
MEDIUM HINGE.
(SEQ ID NO. 80)
EPKSSDKTHTCPPCPAPEAAGG,
LONG HINGE.
(SEQ ID NO. 81)
SLLQHLIGL,
PRAME epitope.
(SEQ ID NO. 82)
ELLGGP,
IgG1 mutation.
PRAME Epitope Binding Bispecific sequences
SEQ ID NO: 83
KAGVTQTPRYLIKTRGQQVTLSCSPIPGHRAVSWYQQTPGQGLQFLFEYVHGEERNKGNFPGRFSGRQFSNSSSE
MNISNLELGDSALYLCASSPWDSPNVQYFGPGTRLTVTEDLKNGSADDAKKDAAKKDGKSQKEVEQNSGPLSVPE
GAIASLNCTYSDRGSQSFFWYRQYSGKSPELIMSIYSNGDKEDGRFTAQLNKASQYVSLLIRDSQPSDSATYLCAAVI
DNDQGGILTFGTGTRLTIIPNIDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALGAPIEKTISKAKGQPREPQVCTLP
PSRDELTKNQVSLSCAVKGFYPSDIAVEWESNGQPENNYKTTPPVLDSDGSFFLVSKLTVDKSRWQQGNVFSCSVM
HEALHNHYTQKSLSLSP, TCE NO10_005, C010_007 scTv{Vb-Kranz-Va}-Fc Hole
Kranz linker underlined italic, hinge underlined.
SEQ ID NO: 84
KAGVTQTPRYLIKTRGQQVTLSCSPIPGHRAVSWYQQTPGQGLQFLFEYVHGEERNKGNFPGRFSGRQFSNSSSE
MNISNLELGDSALYLCASSPWDSPNVQYFGPGTRLTVTEDLKNGGGGSGGGGSGGGGSGGGGSQKEVEQNSGPL
SVPEGAIASLNCTYSDRGSQSFFWYRQYSGKSPELIMSIYSNGDKEDGRFTAQLNKASQYVSLLIRDSQPSDSATYLC
AAVIDNDQGGILTFGTGTRLTIIPNIDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALGAPIEKTISKAKGQPREPQV
CTLPPSRDELTKNQVSLSCAVKGFYPSDIAVEWESNGQPENNYKTTPPVLDSDGSFFLVSKLTVDKSRWQQGNVFSC
SVMHEALHNHYTQKSLSLSP, TCE N010_006, C010_008 scTv{Vb-G4S4-Va}-Fc Hole.
linker underlined italic, hinge underlined.
SEQ ID NO: 85
KAGVTQTPRYLIKTRGQQVTLSCSPIPGHRAVSWYQQTPCQGLQFLFEYVHGEERNKGNFPGRFSGRQFSNSSSEM
NISNLELGDSALYLCASSPWDSPNVQYFGPGTRLTVTEDLKNGGGSGGSGGCPPCGGSGGGKEVEQNSGPLSVPE
GAIASLNCTYSDRGSQSFFWYRQYSGKSPELIMSIYSNGDKEDGRFTAQLNKASQYVSLLIRDSQPSDSATYLCAAVI
DNDQGGILTFGCGTRLTIIPNICPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALGAPIEKTISKAKGQPREPQVCTLPPSRDEL
TKNQVSLSCAVKGFYPSDIAVEWESNGQPENNYKTTPPVLDSDGSFFLVSKLTVDKSRWQQGNVFSCSVMHEALH
NHYTQKSLSLSP, TCE NO10_010, C010_014 spTv{VbG40C-945_CPPC-VaQ01G_T105C}-Fc (shrt) Hole.
linker underlined italic, hinge underlined.
SEQ ID NO: 86
KAGVTQTPRYLIKTRGQQVTLSCSPIPGHRAVSWYQQTPCQGLQFLFEYVHGEERNKGNFPGRFSGRQFSNSSSEM
NISNLELGDSALYLCASSPWDSPNVQYFGPGTRLTVTEDLKNGGGSDDSGGCPPCGGKGGGKEVEQNSGPLSVPE
GAIASLNCTYSDRGSQSFFWYRQYSGKSPELIMSIYSNGDKEDGRFTAQLNKASQYVSLLIRDSQPSDSATYLCAAVI
DNDQGGILTFGCGTRLTIIPNICPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALGAPIEKTISKAKGQPREPQVCTLPPSRDEL
TKNQVSLSCAVKGFYPSDIAVEWESNGQPENNYKTTPPVLDSDGSFFLVSKLTVDKSRWQQGNVFSCSVMHEALH
NHYTQKSLSLSP, TCE NO10_011, C010_015 spTv{VbG40C-945_DDK_CPPC-VaQ01G_T105C}-Fc (shrt)
Hole.
linker underlined italic, hinge underlined.
SEQ ID NO: 87
QKEVEQNSGPLSVPEGAIASLNCTYSDRGSQSFFWYRQYSGKSPELIMSIYSNGDKEDGRFTAQLNKASQYVSLLIRD
SQPSDSATYLCAAVIDNDQGGILTFGTGTRLTIIPNIQNGGGGSGGGGSGGGGSGGGGSKAGVTQTPRYLIKTRGQ
QVTLSCSPIPGHRAVSWYQQTPGQGLQFLFEYVHGEERNKGNFPGRFSGRQFSNSSSEMNISNLELGDSALYLCASS
PWDSPNVQYFGPGTRLTVTEDLKNCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO10_018, C010_022 scTv{Va-G4S4-Vb}-Fc (shrt) Hole.
linker
underlined italic, hinge underlined.
SEQ ID NO: 88
QKEVEQNSGPLSVPEGAIASLNCTYSDRGSQSFFWYRQYSGCSPELIMSIYSNGDKEDGRFTAQLNKASQYVSLLIR
DSQPSDSATYLCAAVIDNDQGGILTFGTGTRLTIIPNIQNGGGSGGSGGCPPCGGSGGKAGVTQTPRYLIKTRGQQV
TLSCSPIPGHRAVSWYQQTPGQGLQFLFEYVHGEERNKGNFPGRFSGRQFSNSSSEMNISNLELGDSALYLCASSP
WDSPNVQYFGCGTRLTVTEDLKNCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO10_014, C010_018 spTv{VaK42C-945_CPPC-VbP106C}-Fc (shrt) Hole.
linker underlined italic, hinge underlined.
SEQ ID NO: 89
QKEVEQNSGPLSVPEGAIASLNCTYSDRGSQSFFWYRQYSGCSPELIMSIYSNGDKEDGRFTAQLNKASQYVSLLIR
DSQPSDSATYLCAAVIDNDQGGILTFGTGTRLTIIPNIQNGGAAGGSGGCPPCGGSGGKAGVTQTPRYLIKTRGQQV
TLSCSPIPGHRAVSWYQQTPGQGLQFLFEYVHGEERNKGNFPGRFSGRQFSNSSSEMNISNLELGDSALYLCASSP
WDSPNVQYFGCGTRLTVTEDLKNCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO10_015, C010_019 spTv{VaK42C-945_AA_CPPC-VbP106C}-Fc (shrt) Hole.
linker underlined italic, hinge underlined.
SEQ ID NO: 90
KAGVTQTPRYLIKTRGQQVTLSCSPIPGHRAVSWYQQTPGQCLQFLFEYVHGEERNKGNFPGRFSGRQFSNSSSEM
NISNLELGDSALYLCASSPWDSPNVQYFGPGTRLTVTEDLKNGGGGSGGGGSGGGGSGGGGSQKEVEQNSGPLSV
PEGAIASLNCTYSDRGSQSFFWYRQYSGKSPELIMSIYSNGDKEDGRFTAQLNKASQYVSLLIRDSQPSDSATYLCAA
VIDNDQGGILTFGCGTRLTIIPNICPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO10_022, C010_025 dsscTv{VbG42C-G4S4-VaT105C}-Fc (shrt) Hole.
linker underlined italic, hinge underlined.
SEQ ID NO: 91
KAGVTQTPRYLIKTRGQQVTLSCSPIPGHRAVSWYQQTPGQGLQFLFEYVHGEERNKGNFPGRFSGRQFSNSSSE
MNISNLELGDSALYLCASSPWDSPNVQYFGPGTRLTVTEDLKNGGGGSGGGGSGGGGSGGGGSQKEVEQNSGPL
SVPEGAIASLNCTYSDRGSQSFFWYRQYSGKSPELIMSIYSNGDKEDGRFTAQLNKASQYVSLLIRDSQPSDSATYLC
AAVIDNDQGGILTFGTGTRLTIIPNIEPKSSDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALGAPIEKTISKAKGQPR
EPQVCTLPPSRDELTKNQVSLSCAVKGFYPSDIAVEWESNGQPENNYKTTPPVLDSDGSFFLVSKLTVDKSRWQQG
NVFSCSVMHEALHNHYTQKSLSLSP, TCE N010_025, C010_029 scTv{Vb-G4S4-Va}-Fc(long) Hole.
linker underlined italic, hinge underlined.
HIV-GAG Binding Bispecific
sequences
SEQ ID NO: 92
EAGVTQSPTHLIKTRGQQVTLRCSPKSGHDTVSWYQQALGQGPQFIFQYVRGEERQRGNFPDRFSGHQYPNYSSE
LNINALLLGDSALYLCASSDTVSYEQYFGPGIRLTVTEDLKNGGGGSGGGGSGGGGGGGGSQKEVEQNSGPLSVP
EGAIASLNCTYSDRGSQSFFWYRQYSGKSPELIMSIYSNGDKEDGRFTAQLNKASQYISLLIRDSKLSDSATYLCAVRG
AHDYALNFGKGTSLLVTPHICPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTISKAKGQPREPQVCTLPPSRDELT
KNQVSLSCAVKGFYPSDIAVEWESNGQPENNYKTTPPVLDSDGSFFLVSKLTVDKSRWQQGNVFSCSVMHEALHN
HYTQKSLSLSP, TCE NO21_009, C021_013 scTv{Z11-Vb-G4S4-Va}-Fc(shrt) Hole.
linker
underlined italic, hinge underlined.
SEQ ID NO: 93
EAGVTQSPTHLIKTRGQQVTLRCSPKSGHDTVSWYQQALCQGPQFIFQYVRGEERQRGNFPDRFSGHQYPNYSSE
LNINALLLGDSALYLCASSDTVSYEQYFGPGIRLTVTEDLKNGGGSGGSGGCPPCGGSGGGKEVEQNSGPLSVPEGAI
ASLNCTYSDRGSQSFFWYRQYSGKSPELIMSIYSNGDKEDGRFTAQLNKASQYISLLIRDSKLSDSATYLCAVRGAHD
YALNFGCGTSLLVTPHICPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALGAPIEKTISKAKGQPREPQVCTLPPSRDELTKNQ
VSLSCAVKGFYPSDIAVEWESNGQPENNYKTTPPVLDSDGSFFLVSKLTVDKSRWQQGNVFSCSVMHEALHNHYT
QKSLSLSP, TCE NO21_010, C021_014 spTv{Z11-VbG40C-945_CPPC-VaQ01G_T105C}-Fc (shrt) Hole.
linker underlined italic, hinge underlined.
SEQ ID NO: 94
QKEVEQNSGPLSVPEGAIASLNCTYSDRGSQSFFWYRQYSGKSPELIMSIYSNGDKEDGRFTAQLNKASQYISLLIRD
SKLSDSATYLCAVRGAHDYALNFGKGTSLLVTPHIQNGGGGSGGGGSGGGGSGGGGSEAGVTQSPTHLIKTRGQQ
VTLRCSPKSGHDTVSWYQQALGQGPQFIFQYVRGEERQRGNFPDRFSGHQYPNYSSELNINALLLGDSALYLCASS
DTVSYEQYFGPGIRLTVTEDLKNCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO21_018, C021_022 scTv{Z11-Va-G4S4-Vb}-Fc (shrt) Hole.
linker underlined italic, hinge underlined.
SEQ ID NO: 95
QKEVEQNSGPLSVPEGAIASLNCTYSDRGSQSFFWYRQYSGCSPELIMSIYSNGDKEDGRFTAQLNKASQYISLLIRD
SKLSDSATYLCAVRGAHDYALNFGKGTSLLVTPHIQNGGGSGGSGGCPPCGGSGGEAGVTQSPTHLIKTRGQQVTL
RCSPKSGHDTVSWYQQALGQGPQFIFQYVRGEERQRGNFPDRFSGHQYPNYSSELNINALLLGDSALYLCASSDTV
SYEQYFGCGIRLTVTEDLKNCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTISKAKGQPREPQVCTLPPSRDELT
KNQVSLSCAVKGFYPSDIAVEWESNGQPENNYKTTPPVLDSDGSFFLVSKLTVDKSRWQQGNVFSCSVMHEALHN
HYTQKSLSLSP, TCE NO21_014, C021_018 spTv{Z11-VaK42C-945_CPPC-VbP105C}-Fc (shrt) Hole.
linker underlined italic, hinge underlined.
HIV-TAX Binding Bispecific
sequences
SEQ ID NO: 96
NAGVTQTPKFQVLKTGQSMTLQCAQDMNHEYMAWYRQDPGMGLRLIHYSVGVGITDQGDVPDGYKVSRSTTE
DFPLRLLSAAPSQTSVYFCASRPGLMSAQPELYFGPGTRLTVTEDLINGSADDAKKDAAKKDGKSQKEVEQNSGPLS
VPEGAIASLNCTYSDRGSQSFFWYRQYSGKSPELIMSIYSNGDKEDGRFTAQLNKASQYVSLLIRDSQPSDSATYLCA
VTTDSWGKLQFGAGTQVVVTPDICPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO22_008, CO22_012 scTv{A6 X15-Vb-Kranz-Va}-Fc(shrt) Hole.
linker underlined italic, hinge underlined.
SEQ ID NO: 97
NAGVTQTPKFQVLKTGQSMTLQCAQDMNHEYMAWYRQDPGMGLRLIHYSVGVGITDQGDVPDGYKVSRSTTE
DFPLRLLSAAPSQTSVYFCASRPGLMSAQPELYFGPGTRLTVTEDLINGGGGSGGGGSGGGGSGGGGSQKEVEQN
SGPLSVPEGAIASLNCTYSDRGSQSFFWYRQYSGKSPELIMSIYSNGDKEDGRFTAQLNKASQYVSLLIRDSQPSDSA
TYLCAVTTDSWGKLQFGAGTQVVVTPDICPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALGAPIEKTISKAKGQPREPQVC
TLPPSRDELTKNQVSLSCAVKGFYPSDIAVEWESNGQPENNYKTTPPVLDSDGSFFLVSKLTVDKSRWQQGNVFSCS
VMHEALHNHYTQKSLSLSP, TCE NO22_009, C022_013 scTv{X15-Vb-G4S4-Va}-Fc(shrt) Hole.
linker underlined italic, hinge underlined.
SEQ ID NO: 98
NAGVTQTPKFQVLKTGQSMTLQCAQDMNHEYMAWYRQDPCMGLRLIHYSVGVGITDQGDVPDGYKVSRSTTE
DFPLRLLSAAPSQTSVYFCASRPGLMSAQPELYFGPGTRLTVTEDLINGGGSGGSGGCPPCGGSGGGKEVEQNSGP
LSVPEGAIASLNCTYSDRGSQSFFWYRQYSGKSPELIMSIYSNGDKEDGRFTAQLNKASQYVSLLIRDSQPSDSATYLC
AVTTDSWGKLQFGCGTQVVVTPDICPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE
ALHNHYTQKSLSLSP, TCE NO22_010, C022_014 spTv{X15-VbG40C-945_CPPC-VaQ01G_T105C}-Fc
(shrt) Hole.
linker underlined italic, hinge underlined.
SEQ ID NO: 99
QKEVEQNSGPLSVPEGAIASLNCTYSDRGSQSFFWYRQYSGKSPELIMSIYSNGDKEDGRFTAQLNKASQYVSLLIRD
SQPSDSATYLCAVTTDSWGKLQFGAGTQVVVTPDIQNGGGGSGGGGSGGGGSGGGGSNAGVTQTPKFQVLKTG
QSMTLQCAQDMNHEYMAWYRQDPGMGLRLIHYSVGVGITDQGDVPDGYKVSRSTTEDFPLRLLSAAPSQTSVYF
CASRPGLMSAQPELYFGPGTRLTVTEDLINCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALGAPIEKTISKAKGQPREPQV
CTLPPSRDELTKNQVSLSCAVKGFYPSDIAVEWESNGQPENNYKTTPPVLDSDGSFFLVSKLTVDKSRWQQGNVFSC
SVMHEALHNHYTQKSLSLSP, TCE NO22_018, C022_022 scTv{X15-Va-G4S4-Vb}-Fc (shrt) Hole.
linker underlined italic, hinge underlined.
SEQ ID NO: 100
QKEVEQNSGPLSVPEGAIASLNCTYSDRGSQSFFWYRQYSGCSPELIMSIYSNGDKEDGRFTAQLNKASQYVSLLIR
DSQPSDSATYLCAVTTDSWGKLQFGAGTQVVVTPDIQNGGGSGGSGGCPPCGGSGGNAGVTQTPKFQVLKTGQS
MTLQCAQDMNHEYMAWYRQDPGMGLRLIHYSVGVGITDQGDVPDGYKVSRSTTEDFPLRLLSAAPSQTSVYFCA
SRPGLMSAQPELYFGCGTRLTVTEDLINCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQVCT
LPPSRDELTKNQVSLSCAVKGFYPSDIAVEWESNGQPENNYKTTPPVLDSDGSFFLVSKLTVDKSRWQQGNVFSCSV
MHEALHNHYTQKSLSLSP, TCE NO22_014, C022_018 spTv{X15-VaK42C-945_CPPC-VbP106C}-Fc (shrt)
Hole.
linker underlined italic, hinge underlined.
MAGE-A4 Binding Bispecific
sequences
SEQ ID NO: 101
DAGVIQSPRHEVTEMGQEVTLRCKPIPGHDYLFWYRQTMMRGLELLFYFCYGTPCDDSGMPEDRFSAKMPNASF
STLKIQPSEPRDSAVYFCASRADTGELFFGEGSRLTVLEDLKNGGGGSGGGGSGGGGSGGGGSQEDVEQSLFLSVR
EGDSVVINCTYTDSSSTYLYWYKQEPGKGLQLLTYIYSSQDQKQDQRLTVLLNKKDKHLSLRIADTQTGDSAIYFCAE
MTSESKIIFGSGTRLSIRPNICPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTISKAKGQPREPQVCTLPPSRDELT
KNQVSLSCAVKGFYPSDIAVEWESNGQPENNYKTTPPVLDSDGSFFLVSKLTVDKSRWQQGNVFSCSVMHEALHN
HYTQKSLSLSP, TCE NO23_009, C023_013 scTv{401-Vb-G4S4-Va}-Fc(shrt) Hole.
linker underlined italic, hinge underlined.
SEQ ID NO: 102
DAGVIQSPRHEVTEMGQEVTLRCKPIPGHDYLFWYRQTMCRGLELLFYFCYGTPCDDSGMPEDRFSAKMPNASFS
TLKIQPSEPRDSAVYFCASRADTGELFFGEGSRLTVLEDLKNGGGSGGSGGCPPCGGSGGGEDVEQSLFLSVREGDS
VVINCTYTDSSSTYLYWYKQEPGKGLQLLTYIYSSQDQKQDQRLTVLLNKKDKHLSLRIADTQTGDSAIYFCAEMTSES
KIIFGCGTRLSIRPNICPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALGAPIEKTISKAKGQPREPQVCTLPPSRDELTKNQVSL
SCAVKGFYPSDIAVEWESNGQPENNYKTTPPVLDSDGSFFLVSKLTVDKSRWQQGNVFSCSVMHEALHNHYTQKS
LSLSP, TCE NO23_010, CO23_014 spTv{401-VbG40C-945_CPPC-VaQ01G_S102C}-Fc (shrt) Hole.
linker underlined italic, hinge underlined.
SEQ ID NO: 103
GEDVEQSLFLSVREGDSVVINCTYTDSSSTYLYWYKQEPGKGLQLLTYIYSSQDQKQDQRLTVLLNKKDKHLSLRIADT
QTGDSAIYFCAEMTSESKIIFGSGTRLSIRPNIQNGGGGSGGGGSGGGGSGGGGSDAGVIQSPRHEVTEMGQEVTL
RCKPIPGHDYLFWYRQTMMRGLELLFYFCYGTPCDDSGMPEDRFSAKMPNASFSTLKIQPSEPRDSAVYFCASRAD
TGELFFGEGSRLTVLEDLKNCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTISKAKGQPREPQVCTLPPSRDELT
KNQVSLSCAVKGFYPSDIAVEWESNGQPENNYKTTPPVLDSDGSFFLVSKLTVDKSRWQQGNVFSCSVMHEALHN
HYTQKSLSLSP, TCE NO23_018, C023_022 scTv{401-Va-G4S4-Vb}-Fc (shrt) Hole.
linker
underlined italic, hinge underlined.
SEQ ID NO: 104
GEDVEQSLFLSVREGDSVVINCTYTDSSSTYLYWYKQEPGCGLQLLTYIYSSQDQKQDQRLTVLLNKKDKHLSLRIADT
QTGDSAIYFCAEMTSESKIIFGSGTRLSIRPNIQNGGGSGGSGGCPPCGGSGGDAGVIQSPRHEVTEMGQEVTLRC
KPIPGHDYLFWYRQTMMRGLELLFYFCYGTPCDDSGMPEDRFSAKMPNASFSTLKIQPSEPRDSAVYFCASRADTG
ELFFGCGSRLTVLEDLKNCPPCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALGAPIEKTISKAKGQPREPQVCTLPPSRDELTKN
QVSLSCAVKGFYPSDIAVEWESNGQPENNYKTTPPVLDSDGSFFLVSKLTVDKSRWQQGNVFSCSVMHEALHNHY
TQKSLSLSP, TCE NO23_014, C023_018 spTv{401-VaK42C-945_CPPC-VbP106C}-Fc (shrt) Hole.
linker underlined italic, hinge underlined.
scFv Chain
sequences for PRAME TCE Medium/Long Hinge
SEQ ID NO: 105
DIQMTQSPSSLSASVGDRVTITCRASQDIRNYLNWYQQKPGKAPKLLIYYTSRLESGVPSRFSGSGSGTDYTLTISSLQ
PEDFATYYCQQGNTLPWTFGQGTKVEIKGGGGSGGGGSGGGGSGGGGSEVQLVESGGGLVQPGGSLRLSCAASG
YSFTGYTMNWVRQAPGKGLEWVALINPYKGVSTYNQKFKDRFTISVDKSKNTAYLQMNSLRAEDTAVYYCARSGYY
GDSDWYFDVWGQGTLVTVSSDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQVYTL
PPCRDELTKNQVSLWCLVKGFYPSDIAVEWESNGQPENNYKTTPPVLDSDGSFFLYSKLTVDKSRWQQGNVFSCSV
MHEALHNHYTQKSLSLSP, TCE NO10_006, C010_006 scFv{VL-G4S4-VH}-Fc Knob.
linker underlined italic, hinge underlined.
SEQ ID NO: 106
DIQMTQSPSSLSASVGDRVTITCRASQDIRNYLNWYQQKPGKAPKLLIYYTSRLESGVPSRFSGSGSGTDYTLTISSLQ
PEDFATYYCQQGNTLPWTFGQGTKVEIKGGGGSGGGGSGGGGSGGGGSEVQLVESGGGLVQPGGSLRLSCAASG
YSFTGYTMNWVRQAPGKGLEWVALINPYKGVSTYNQKFKDRFTISVDKSKNTAYLQMNSLRAEDTAVYYCARSGYY
GDSDWYFDVWGQGTLVTVSSEPKSSDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALGAPIEKTISKAKGQPREP
QVYTLPPCRDELTKNQVSLWCLVKGFYPSDIAVEWESNGQPENNYKTTPPVLDSDGSFFLYSKLTVDKSRWQQGNV
FSCSVMHEALHNHYTQKSLSLSP, TCE NO10_025, C010_028 scFv{VL-G4S4-VH}-Fc(long) Knob.
linker underlined italic, hinge underlined.
TCER MAGE A4
sequences
SEQ ID NO: 107
EDVEQSLFLSVREGDSVVINCTYTDSSSTYLYWYKQEPGKGLQLLTYIYSSQDQKQDQRLTVLLNKKDKHLSLRIADTQ
TGDSAIYFCAEMTSESKIIFGSGTRLSIRPGGGSGGGGDIQMTQSPSSLSASVGDRVTITCRASQDIRNYLNWYQQK
PGKAPKLLIYYTSRLHSGVPSRFSGSGSGTDYTLTISSLQPEDIATYFCQQGQTLPWTFGQGTKVEIKEPKSSDKTHTC
PPCPAPPVAGPSVFLFPPKPKDTLMISRTPEVTCVVVDVSHEDPEVKFNWYVDGVEVHNAKTKPREEQYQSTYRVV
SVLTVLHQDWLNGKEYKCKVSNKALPASIEKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVMHEALHNHYTQKSLSLSP, TCER
NO11_034 (TCER vs MAG-A4), C011_003 Va(R7P1D5_114-iso1)-G3SG4-UCHT1(V17)VL-Fc Knob
TCER.
CDRs underlined.
SEQ ID NO: 108
EVQLVQSGAEVKKPGASVKVSCKASGYSFTGYTMNWVRQAPGQGLEWMGLINPYKGVSTYAQKFQDRVTLTVDK
STSTAYMELSSLRSEDTAVYYCARSGYYGDSDWYFDVWGQGTLVTVSSGGGSGGGGDAGVIQSPRHEVTEMGQE
VTLRCKPIPGHDYLFWYRQTMMRGLELLFYFCYGTPCDDSGMPEDRFSAKMPNASFSTLKIQPSEPRDSAVYFCAS
RADTGELFFGEGSRLTVLEPKSSDKTHTCPPCPAPPVAGPSVFLFPPKPKDTLMISRTPEVTCVVVDVSHEDPEVKFN
WYVDGVEVHNAKTKPREEQYQSTYRVVSVLTVLHQDWLNGKEYKCKVSNKALPASIEKTISKAKGQPREPQVCTLP
PSRDELTKNQVSLSCAVKGFYPSDIAVEWESNGQPENNYKTTPPVLDSDGSFFLVSKLTVDKSRWQQGNVFSCSVM
HEALHNHYTQKSLSLSP, TCER NO11_034 (TCER vs MAG-A4), C011_052 UCHT1(V17)VH-G3SG4-
Vb(R7P1D5_114-iso1)-Fc Hole TCER.
CDRs underlined.
CDR SEQUENCES OF TCE SCTVs
HIV-GAG scTv CDRs
CDR1 Alpha
SEQ ID NO: 110
DRGSQ,
CDR2 Alpha
SEQ ID NO: 111
IYSN,
CDR3 Alpha
SEQ ID NO: 112
RGAHDYA,
CDR1 Beta
SEQ ID NO: 113
KSGHD,
CDR2 Beta
SEQ ID NO: 114
VRGEE,
CDR3 Beta
SEQ ID NO: 115
DTVSY,
HIV-TAX scTv CDRs,
CDR1 Alpha
SEQ ID NO: 116
DRGSQ,
CDR2 Alpha
SEQ ID NO: 117
IYSN,
CDR3 Alpha
SEQ ID NO: 118
TTDSWGK,
CDR1 Beta
SEQ ID NO: 119
DMNHE,
CDR2 Beta
SEQ ID NO: 120
VGVGI,
CDR3 Beta
SEQ ID NO: 121
PGLMSAQP,
MAGE-A4 scTv CDRs,
CDR1 Alpha
SEQ ID NO: 122
DSSST,
CDR2 Alpha
SEQ ID NO: 123
YIYSS,
CDR3 Alpha
SEQ ID NO: 124
MTSESKII,
CDR1 Beta
SEQ ID NO: 125
IPGHD,
CDR2 Beta
SEQ ID NO: 126
CYGTPC,
CDR3 Beta
SEQ ID NO: 127
ADTGELFFG,
PEPTIDE EPITOPES
SEQ ID NO: 128
SLYNTVATL,
HIV-GAG peptide
SEQ ID NO: 129
LLFGYPVYV,
HIV-TAX peptide.
SEQ ID NO: 130
KVLEHVVRVI,
MAGE-A4 peptide.
CONNECTORS
SEQ ID NO. 131
NICC,
SHORT ANTIGEN BINDING DOMAIN CONNECTOR SEQUENCE,
(P)-N-I-C═C-(S)
where (P) and (S) are the first residues of the scTv Valpha and scFv VH respectively.
SEQ ID NO. 132
NIDKTHTCCTHTKD,
MEDIUM ANTIGEN BINDING DOMAIN CONNECTOR SEQUENCE,
(P)-NIDKTHTC═CTHTKD-(S).
SEQ ID NO. 133
NIEPKSSDKTHTCCTHTKDSSKPE,
LONG ANTIGEN BINDING DOMAIN CONNECTOR
SEQUENCE, (P)-NIEPKSSDKTHTC═CTHTKDSSKPE-(S).
PRAME TCER
SEQUENCES
SEQ ID NO. 134
EVQLVESGGGLVQPGGSLRLSCAASGYSFTGYTMNWVRQAPGKGLEWVALINPYKGVSTYNQKFKDRFTISVDKSK
NTAYLQMNSLRAEDTAVYYCARSGYYGDSDWYFDVWGQGTLVTVSSGGGSGGGGKAGVTQTPRYLIKTRGQQVT
LSCSPIPGHRAVSWYQQTPGQGLQFLFEYVHGEERNKGNFPGRFSGRQFSNSSSEMNISNLELGDSALYLCASSPW
DSPNVQYFGPGTRLTVTEDLKNEPKSSDKTHTCPPCPAPPVAGPSVFLFPPKPKDTLMISRTPEVTCVVVDVSHEDPE
VKFNWYVDGVEVHNAKTKPREEQYQSTYRVVSVLTVLHQDWLNGKEYKCKVSNKALPASIEKTISKAKGQPREPQV
CTLPPSRDELTKNQVSLSCAVKGFYPSDIAVEWESNGQPENNYKTTPPVLDSDGSFFLVSKLTVDKSRWQQGNVFSC
SVMHEALHNHYTQKSLSLSPGK,
N011_019,
C011_032 UCHT1(v09)VH-G3SG4-Vb(PRAME)-Fc Hole.
SEQ ID NO. 135
QKEVEQNSGPLSVPEGAIASLNCTYSDRGSQSFFWYRQYSGKSPELIMSIYSNGDKEDGRFTAQLNKASQYVSLLIRD
SQPSDSATYLCAAVIDNDQGGILTFGTGTRLTIIPNIQNGGGSGGGGDIQMTQSPSSLSASVGDRVTITCRASQDIRN
YLNWYQQKPGKAPKLLIYYTSRLESGVPSRFSGSGSGTDYTLTISSLQPEDFATYYCQQGNTLPWTFGQGTKVEIKEP
KSSDKTHTCPPCPAPPVAGPSVFLFPPKPKDTLMISRTPEVTCVVVDVSHEDPEVKFNWYVDGVEVHNAKTKPREE
QYQSTYRVVSVLTVLHQDWLNGKEYKCKVSNKALPASIEKTISKAKGQPREPQVYTLPPCRDELTKNQVSLWCLVKG
FYPSDIAVEWESNGQPENNYKTTPPVLDSDGSFFLYSKLTVDKSRWQQGNVFSCSVMHEALHNHYTQKSLSLSPGK,
N011_019,
C011_025 Va(PRAME)-G3SG4-UCHT1(v09)VL-Fc Knob.
Va-Vb Orientation-C is inserted at Va 42 (IMGT 47) and Vb 106 (IGMT 120)
SEQ ID NO: 136
QKEVEQNSGPLSVPEGAIASLNCTYSDRGSQSFFWYRQYSCKSPELIMSIYSNGDKEDGRFTAQLNKASQYVSLLIRD
SQPSDSATYLCAAVIDNDQGGILTFGTGTRLTIIPNIQNGGGSGGSGGCPPCGGSGGKAGVTQTPRYLIKTRGQQVT
LSCSPIPGHRAVSWYQQTPGQGLQFLFEYVHGEERNKGNFPGRFSGRQFSNSSSEMNISNLELGDSALYLCASSPW
DSPNVQYFGCGTRLTVTEDLKNCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE,
ALHNHYTQKSLSLSP, N010_014A, C010_018A spTv{VaK42C-945_CPPC-VbP106C}-Fc (shrt) Hole.
PRAME scTv, linker italic underlined, hinge underlined, substituted cysteine underlined
bold.
SEQ ID NO: 137
QKEVEQNSGPLSVPEGAIASLNCTYSDRGSQSFFWYRQYSCKSPELIMSIYSNGDKEDGRFTAQLNKASQYVSLLIRD
SQPSDSATYLCAAVIDNDQGGILTFGTGTRLTIIPNIQNGGAAGGSGGCPPCGGSGGKAGVTQTPRYLIKTRGQQVT
LSCSPIPGHRAVSWYQQTPGQGLQFLFEYVHGEERNKGNFPGRFSGRQFSNSSSEMNISNLELGDSALYLCASSPW
DSPNVQYFGCGTRLTVTEDLKNCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE,
ALHNHYTQKSLSLSP, NO10_015A, C010_019A spTv{VaK42C-945_AA_CPPC-VbP106C}-Fc (shrt) Hole.
PRAME scTv, linker italic underlined, hinge underlined, substituted cysteine
underlined bold.
SEQ ID NO: 138
QKEVEQNSGPLSVPEGAIASLNCTYSDRGSQSFFWYRQYSCKSPELIMSIYSNGDKEDGRFTAQLNKASQYISLLIRD
SKLSDSATYLCAVRGAHDYALNFGKGTSLLVTPHIQNGGGSGGSGGCPPCGGSGGEAGVTQSPTHLIKTRGQQVTL
RCSPKSGHDTVSWYQQALGQGPQFIFQYVRGEERQRGNFPDRFSGHQYPNYSSELNINALLLGDSALYLCASSDTV
SYEQYFGCGIRLTVTEDLKNCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTISKAKGQPREPQVCTLPPSRDELT
KNQVSLSCAVKGFYPSDIAVEWESNGQPENNYKTTPPVLDSDGSFFLVSKLTVDKSRWQQGNVFSCSVMHEALHN
HYTQKSLSLSP, NO21_014A, C021_018A spTv{Z11-VaK42C-945_CPPC-VbP105C}-Fc (shrt) Hole.
HIV-GAG scTv, linker italic underlined, hinge underlined, substituted cysteine underlined
bold.
SEQ ID NO: 139
QKEVEQNSGPLSVPEGAIASLNCTYSDRGSQSFFWYRQYSCKSPELIMSIYSNGDKEDGRFTAQLNKASQYVSLLIRD
SQPSDSATYLCAVTTDSWGKLQFGAGTQVVVTPDIQNGGGSGGSGGCPPCGGSGGNAGVTQTPKFQVLKTGQS
MTLQCAQDMNHEYMAWYRQDPGMGLRLIHYSVGVGITDQGDVPDGYKVSRSTTEDFPLRLLSAAPSQTSVYFCA
SRPGLMSAQPELYFGCGTRLTVTEDLINCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQVCT
LPPSRDELTKNQVSLSCAVKGFYPSDIAVEWESNGQPENNYKTTPPVLDSDGSFFLVSKLTVDKSRWQQGNVFSCSV,
MHEALHNHYTQKSLSLSP, NO22_014A, C022_018A spTv{X15-VaK42C-945_CPPC-VbP106C}-Fc (shrt)
Hole.
HIV-TAX scTv, linker italic underlined, hinge underlined, substituted cysteine
underlined bold.
SEQ ID NO: 140
GEDVEQSLFLSVREGDSVVINCTYTDSSSTYLYWYKQEPCKGLQLLTYIYSSQDQKQDQRLTVLLNKKDKHLSLRIADT
QTGDSAIYFCAEMTSESKIIFGSGTRLSIRPNIQNGGGSGGSGGCPPCGGSGGDAGVIQSPRHEVTEMGQEVTLRC
KPIPGHDYLFWYRQTMMRGLELLFYFCYGTPCDDSGMPEDRFSAKMPNASFSTLKIQPSEPRDSAVYFCASRADTG
ELFFGCGSRLTVLEDLKNCPPCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALGAPIEKTISKAKGQPREPQVCTLPPSRDELTKN
QVSLSCAVKGFYPSDIAVEWESNGQPENNYKTTPPVLDSDGSFFLVSKLTVDKSRWQQGNVFSCSVMHEALHNHY
TQKSLSLSP, NO23_014A, C023_018A spTv{401-VaK42C-945_CPPC-VbP106C}-Fc (shrt) Hole.
MAGE-A4 scTv, linker italic underlined, hinge underlined, substituted cysteine underlined
bold.
Vb-Va Chains-C is inserted at Vb 39 (IGMT 46) and Va 107 (IMGT 120)
SEQ ID NO: 141
KAGVTQTPRYLIKTRGQQVTLSCSPIPGHRAVSWYQQTCGQGLQFLFEYVHGEERNKGNFPGRFSGRQFSNSSSE
MNISNLELGDSALYLCASSPWDSPNVQYFGPGTRLTVTEDLKNGGGSGGSGGCPPCGGSGGGKEVEQNSGPLSVP
EGAIASLNCTYSDRGSQSFFWYRQYSGKSPELIMSIYSNGDKEDGRFTAQLNKASQYVSLLIRDSQPSDSATYLCAAVI
DNDQGGILTFGCGTRLTIIPNICPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALGAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSVMHEALH,
NHYTQKSLSLSP, NO10_010A, C010_014A spTv{VbG40C-945_CPPC-VaQ01G_T105C}-Fc (shrt) Hole.
PRAME scTv, , linker italic underlined, hinge underlined, substituted cysteine
underlined bold.
SEQ ID NO: 142
KAGVTQTPRYLIKTRGQQVTLSCSPIPGHRAVSWYQQTCGQGLQFLFEYVHGEERNKGNFPGRFSGRQFSNSSSE
MNISNLELGDSALYLCASSPWDSPNVQYFGPGTRLTVTEDLKNGGGSDDSGGCPPCGGKGGGKEVEQNSGPLSVP
EGAIASLNCTYSDRGSQSFFWYRQYSGKSPELIMSIYSNGDKEDGRFTAQLNKASQYVSLLIRDSQPSDSATYLCAAVI
DNDQGGILTFGCGTRLTIIPNICPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALGAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSVMHEALH,
NHYTQKSLSLSP, NO10_011A, C010_015A spTv{VbG40C-945_DDK_CPPC-VaQ01G_T105C}-Fc (shrt)
Hole.
PRAME scTv, linker italic underlined, hinge underlined, substituted cysteine
underlined bold.
SEQ ID NO: 143
EAGVTQSPTHLIKTRGQQVTLRCSPKSGHDTVSWYQQACGQGPQFIFQYVRGEERQRGNFPDRFSGHQYPNYSSE
LNINALLLGDSALYLCASSDTVSYEQYFGPGIRLTVTEDLKNGGGSGGSGGCPPCGGSGGGKEVEQNSGPLSVPEGAI
ASLNCTYSDRGSQSFFWYRQYSGKSPELIMSIYSNGDKEDGRFTAQLNKASQYISLLIRDSKLSDSATYLCAVRGAHD
YALNFGCGTSLLVTPHICPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALGAPIEKTISKAKGQPREPQVCTLPPSRDELTKNQ
VSLSCAVKGFYPSDIAVEWESNGQPENNYKTTPPVLDSDGSFFLVSKLTVDKSRWQQGNVFSCSVMHEALHNHYT,
QKSLSLSP NO21_010A, C021_014A spTv{Z11-VbG40C-945_CPPC-VaQ01G_T105C}-Fc (shrt) Hole.
HIV-GAG scTv, linker italic underlined, hinge underlined, substituted cysteine
underlined bold.
SEQ ID NO: 144
NAGVTQTPKFQVLKTGQSMTLQCAQDMNHEYMAWYRQDCGMGLRLIHYSVGVGITDQGDVPDGYKVSRSTTE
DFPLRLLSAAPSQTSVYFCASRPGLMSAQPELYFGPGTRLTVTEDLINGGGSGGSGGCPPCGGSGGGKEVEQNSGP
LSVPEGAIASLNCTYSDRGSQSFFWYRQYSGKSPELIMSIYSNGDKEDGRFTAQLNKASQYVSLLIRDSQPSDSATYLC
AVTTDSWGKLQFGCGTQVVVTPDICPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALGAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMHE,
ALHNHYTQKSLSLSP, NO22_010A, C022_014A spTv{X15-VbG40C-945_CPPC-VaQ01G_T105C}-Fc (shrt)
Hole.
HIV-TAX scTv, linker italic underlined, hinge underlined, substituted cysteine
underlined bold.
SEQ ID NO: 145
DAGVIQSPRHEVTEMGQEVTLRCKPIPGHDYLFWYRQTCMRGLELLFYFCYGTPCDDSGMPEDRFSAKMPNASFS
TLKIQPSEPRDSAVYFCASRADTGELFFGEGSRLTVLEDLKNGGGSGGSGGCPPCGGSGGGEDVEQSLFLSVREGDS
VVINCTYTDSSSTYLYWYKQEPGKGLQLLTYIYSSQDQKQDQRLTVLLNKKDKHLSLRIADTQTGDSAIYFCAEMTSES
KIIFGCGTRLSIRPNICPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALGAPIEKTISKAKGQPREPQVCTLPPSRDELTKNQVSL
SCAVKGFYPSDIAVEWESNGQPENNYKTTPPVLDSDGSFFLVSKLTVDKSRWQQGNVFSCSVMHEALHNHYTQKS,
LSLSP, NO23_010A, C023_014A spTv{401-VbG40C-945_CPPC-VaQ01G_S102C}-Fc (shrt) Hole.
MAGE-A4 scTv, linker italic underlined, hinge underlined, substituted cysteine underlined
bold.
SEQ ID NO. 146
YSGCSPE.
V alpha anchor,
SEQ ID NO. 147
EPGCGLQ.
V alpha anchor,
SEQ ID NO. 148
YFGCGTR.
V beta anchor,
SEQ ID NO. 149
FFGCGSR.
V beta anchor,
SEQ ID NO. 150
YSCKSPE.
V alpha anchor,
SEQ ID NO. 151
EPCKGLQ.
V alpha anchor,
SEQ ID NO. 152
YFGCGTR.
V beta anchor,
SEQ ID NO. 153
FFGCGSR.
V beta anchor,
SEQ ID NO. 154
QTPCQGL.
V beta anchor,
SEQ ID NO. 155
QALCQGP.
V beta anchor,
SEQ ID NO. 156
QDPCMGL.
V beta anchor,
SEQ ID NO. 157
QTMCRGL.
V beta anchor,
SEQ ID NO. 158
TFGCGTR.
V alpha anchor,
SEQ ID NO. 159
NFGCGTS.
V alpha anchor,
SEQ ID NO. 160
QFGCGTQ.
V alpha anchor,
SEQ ID NO. 161
IFGCGTR.
V alpha anchor,
SEQ ID NO. 162
QTCGQGL.
V beta anchor,
SEQ ID NO. 163
QACGQGP.
V beta anchor,
SEQ ID NO. 164
QDCGMGL.
V beta anchor,
SEQ ID NO. 165
QTCMRGL.
V beta anchor,
SEQ ID NO. 166
QTCMRGL.
V beta anchor,
UCHT1.v17 Sequences
SEQ ID NO. 167
DIQMTQSPSSLSASVGDRVTITCRASQDIRNYLNWYQQKPGKAPKLLIYYTSRLHSGVPSRFSGSGSGTDYTLTISSLQ
PEDIATYFCQQGQTLPWTFGQGTKVEIK. (UCHT1.v17. VL)
CDR 1, 2,3 respectively underlined.
SEQ ID NO. 168
EVQLVQSGAEVKKPGASVKVSCKASGYSFTGYTMNWVRQAPGQGLEWMGLINPYKGVSTYAQKFQDRVTLTVDK
STSTAYMELSSLRSEDTAVYYCARSGYYGDSDWYFDVWGQGTLVTV. (UCHT1.v17. VH)
CDR 1, 2, 3 respectively underlined.
HIV-GAG scTv Vα, Vβ
SEQ ID NO. 169
QKEVEQNSGPLSVPEGAIASLNCTYSDRGSQSFFWYRQYSGKSPELIMSIYSNGDKEDGRFTAQLNKASQYISLLIRD
SKLSDSATYLCAVRGAHDYALNFGKGTSLLVTPHI,
(Tv alpha (HIV-GAG), [Vα]). CDRs underlined.
Q at position 1 may be replaced by G in some embodiments.
SEQ ID NO. 170
EAGVTQSPTHLIKTRGQQVTLRCSPKSGHDTVSWYQQALGQGPQFIFQYVRGEERQRGNFPDRFSGHQYPNYSSE
LNINALLLGDSALYLCASSDTVSYEQYFGPGIRLTVTEDLKN,
(Tv beta (HIV-GAG), [Vβ]). CDRs underlined.
HIV-TAX scTv Vα, Vβ
SEQ ID NO. 171
QKEVEQNSGPLSVPEGAIASLNCTYSDRGSQSFFWYRQYSGKSPELIMSIYSNGDKEDGRFTAQLNKASQYVSLLIRD
SQPSDSATYLCAVTTDSWGKLQFGAGTQVVVTPDI,
(Tv alpha (HIV-TAX), [Vα]). CDRs underlined.
Q at position 1 may be replaced by G in some embodiments.
SEQ ID NO. 172
NAGVTQTPKFQVLKTGQSMTLQCAQDMNHEYMAWYRQDPGMGLRLIHYSVGVGITDQGDVPDGYKVSRSTTE
DFPLRLLSAAPSQTSVYFCASRPGLMSAQPELYFGPGTRLTVTEDLIN,
(Tv beta (HIV-TAX), [Vβ]). CDRs underlined.
MAGE-A4 scTv Vα, Vβ
SEQ ID NO. 173
QEDVEQSLFLSVREGDSVVINCTYTDSSSTYLYWYKQEPGKGLQLLTYIYSSQDQKQDQRLTVLLNKKDKHLSLRIADT
QTGDSAIYFCAEMTSESKIIFGSGTRLSIRPNI,
(Tv alpha (MAGE-A4), [Vα]). CDRs underlined.
Q at position 1 may be replaced by G in some embodiments.
SEQ ID NO. 174
DAGVIQSPRHEVTEMGQEVTLRCKPIPGHDYLFWYRQTMMRGLELLFYFCYGTPCDDSGMPEDRFSAKMPNASF
STLKIQPSEPRDSAVYFCASRADTGELFFGEGSRLTVLEDLKN,
(Tv beta (HIV-TAX), [Vβ]).
CDRs underlined.
SEQ ID NO. 175
C═C,
CNX sequence, where “═”represents a disulphide bond.
SEQ ID NO. 176
TC═CT,
CNX sequence, where “═”represents a disulphide bond.
SEQ ID NO. 177
HTC═CTH,
CNX sequence, where “═”represents a disulphide bond.
SEQ ID NO. 178
THTC═CTHT,
CNX sequence, where “═”represents a disulphide bond.
SEQ ID NO. 179
KTHTC═CTHTK,
CNX sequence, where “═”represents a disulphide bond.
SEQ ID NO. 180
DKTHTC═CTHTKD,
CNX sequence, where “═”represents a disulphide bond.
SEQ ID NO. 181
EPKSSDKTHTC═CTHTKDSSKPE,
CNX sequence, where “═”represents a disulphide
bond.
(SEQ ID NO. 182)
NI,
Cα residues (PRAME/MAGE-A4).
(SEQ ID NO. 183)
HI,
Cα residues (HIV-GAG).
(SEQ ID NO. 184)
DI,
Cα residues (HIV-TAX).
(SEQ ID NO. 185)
NIQN,
Cα residues (PRAME/MAGE-A4).
(SEQ ID NO. 186)
HIQN,
Cα residues (HIV-GAG).
(SEQ ID NO. 187)
DIQN,
Cα residues (HIV-TAX).
(SEQ ID NO. 188)
QN,
Cα residues.
(SEQ ID NO. 189)
EDLKN,
CB residues.
(SEQ ID NO. 190)
EDLIN,
CB residues.

1. A bispecific polypeptide molecule capable of simultaneously binding a first and second antigen, wherein the bispecific polypeptide molecule comprises a first polypeptide chain and a second polypeptide chain, wherein:
(A) the first polypeptide chain comprises: (i) a first antigen-binding domain, (ii) a first hinge domain subunit, (iii) a first Fc domain subunit; and
(B) the second polypeptide chain comprises: (i) a second antigen-binding domain, ii) a second hinge domain subunit, (iii) a second Fc domain subunit;
wherein the first and second hinge domain subunits are capable of forming a stable association as a hinge domain and the first and second Fc domain subunits are capable of forming a stable association as an Fc domain or Fc domain portion, such that the two polypeptide chains are connected by covalent and/or non-covalent bonds between the hinge domain subunits and Fc-domain subunits, preferably wherein the first and second antigens are expressed on two distinct cells.

2.-4. (canceled)

5. The bispecific polypeptide molecule according to claim 1, wherein the first antigen-binding domain comprises a linker LNK1 connecting a first binding region of a variable domain (VD1′) of an antibody and a second binding region of a variable domain (VD2) of an antibody, wherein the linker LNK1 is a flexible linker of between 3 and 20 amino acids.

6.-9. (canceled)

10. The bispecific polypeptide molecule according to claim 1, wherein the first antigen-binding domain comprises or consists of a single chain Fv (scFv).

11.-16. (canceled)

17. The bispecific polypeptide molecule according to claim 1, wherein the second antigen-binding domain comprises a linker LNK2 connecting a first binding region of a variable domain (VR1) of an antibody and a second binding region of a variable domain (VR2) of an antibody, wherein the linker LNK2 is a flexible linker of between 3 and 20 amino acids.

18. The bispecific polypeptide molecule according to claim 17, wherein the VR1comprises a TCR α chain variable domain (Vα) or antigen or MHC-associated peptide epitope binding portion thereof and the VR2comprises a TCR β chain variable domain (Vβ) or antigen or MHC-associated peptide epitope binding portion thereof, optionally wherein the VR1may further comprise part or all of a TCR α chain constant domain, Cα, preferably linked to or fused to the C-terminus of the Vα domain and/or the VR2may further comprise part or all of TCR β chain constant domain, Cβ, preferably linked to or fused to the C-terminus of the Vβ domain.

19.-24. (canceled)

25. The bispecific polypeptide molecule according to claim 1, wherein the second antigen binding domain comprises or consists of a single chain TCR variable domain (scTv).

26. The bispecific polypeptide molecule according to claim 1, wherein the second antigen binding domain or the second antigen binding site (VR1)(VR2) binds or specifically binds the second antigen with an affinity (KD) of about 100 M or less.

27. The bispecific polypeptide molecule according to claim 26, wherein the second antigen is an MHC-associated peptide epitope or antigen (pMHC).

28. The bispecific polypeptide molecule according to claim 26, wherein the second antigen is a cancer or tumour cell antigen and/or is detectibly present on the cell surface or a cancer or tumour cell and/or the MHC-associated peptide epitope or antigen is a cancer or tumour cell epitope or antigen and/or is detectibly present on the cell surface or a cancer or tumour cell.

29. The bispecific polypeptide molecule according to claim 26, wherein the binding to said second antigen or MHC-associated peptide epitope or antigen, results in immune effector cell activation.

30. (canceled)

31. The bispecific polypeptide molecule according to claim 1, wherein the first hinge domain subunit is fused to the first antigen-binding domain and the second hinge domain subunit is fused to the second antigen-binding domain, wherein the fusion orientates the antigen-binding domain at the N-terminal and the hinge domain subunit at the C-terminal and wherein the fusion is by means of a peptide bond between the C-terminal amino acid of the respective antigen binding domain and the N-terminal amino acid of hinge domain subunit.

32. The bispecific polypeptide molecule according to claim 31, wherein the first hinge domain subunit and the second hinge domain subunit is between 2 and 25 amino acids in length.

33.-38. (canceled)

39. The bispecific polypeptide molecule according to claim 1, wherein the distance between the first antigen binding domain and the second antigen binding domain is any of about 1.36, 1.74, 2.12, 2.5, 2.88, 3.26, 3.64, 4.02, 4.4, 4.78, 5.16, 5.54, 5.92, 6.3, 6.68, 7.06, 7.44, 7.82, 8.2, 8.58, 8.96, 9.34 or about 9.72 nm, said distances in nm being the sum of alpha-carbon to alpha-carbon distances plus the distance of between the two alpha carbons of the first and second cysteine residues.

40. The bispecific polypeptide molecule according to claim 1, wherein the Fc domain is derived from human IgG1, IgG2 or IgG4 Fc domain or a portion thereof, or a chimera of any two or three of an IgG1, IgG2 or IgG4 Fc domain or a portion thereof, preferably wherein the portion is a dimerising portion.

41. The bispecific polypeptide molecule according to claim 40, wherein the Fc domain exhibits reduced binding affinity to an Fc receptor and/or reduced effector function, as compared to a native IgG1, IgG2 or IgG4 Fc domain and/or wherein the Fc domain comprises one or more amino acid substitutions that reduce binding to an Fc receptor and/or effector function as compared to a native IgG1, IgG2 or IgG4 Fc domain.

42. The bispecific polypeptide molecule according to claim 40, wherein the Fc domain comprises at least one effector function silencing mutation at a residue selected from positions 233, 234, 235, 236, 297 and 331 and/or wherein the effector function silencing mutation is generated by replacing at least one residue in position 233, 234, 235, 236, and 331 (EU numbering).

43. The bispecific polypeptide molecule according to claim 40, wherein in a CH3 domain of the first Fc domain subunit or portion thereof comprises a sequence in which an amino acid residue is replaced with an amino acid residue having a larger side chain volume, thereby generating a protuberance within the CH3 domain of the first Fc domain subunit or portion thereof which is positionable in a cavity within a CH3 domain of the second Fc domain subunit or portion thereof, and which in the CH3 domain of the second Fc domain subunit or portion thereof an amino acid residue is replaced with an amino acid residue having a smaller side chain volume, thereby generating a cavity within the CH3 domain of the second Fc domain subunit or portion thereof within which the protuberance within the CH3 domain of the first subunit is positionable, preferably wherein the formation of heterodimers is facilitated.

44.-55. (canceled)

56. A method of treatment of cancer in a subject in need thereof comprising administering to the subject an effective amount of the bispecific polypeptide molecule according to claim 1.

57. (canceled)

58. A method of eliciting an immune response in a subject in need thereof comprising administering to the subject an effective amount of the bispecific polypeptide molecule according to claim 1.

59. A method of engaging an immune effector cell with a cell target in a subject in need thereof comprising administering to the subject an effective amount of the bispecific polypeptide molecule according to claim 1, wherein the first antigen binding domain specifically binds the cell target and the second antigen binding domain specifically binds the immune effector cell.
